(19) United States
(12) Patent Application Publication
(10) Pub. No.: US 2013/0344143 A1
(43) Pub. Date: Dec. 26, 2013

(54) FORMULATIONS OF NONOPIOID AND CONFINED OPIOID ANALGESICS

(71) Applicants: AbbVie Deutschland GmbH & Co. KG, (US); AbbVie Inc., (US)

(72) Inventors: Joerg Rosenberg, Ellerstadt (DE); Gerd Woehrle, Eppelheim (DE); Thomas Y. Kessler, Schifferstadt (DE); Joerg Breitenbach, Mannheim (DE); Salih Durak, Ludwigshafen (DE); Friedrich W. Richter, Lake Bluff, IL (US); Wei Liu, Mundelein, IL (US); Sandeep Dutta, Gurnee, IL (US)

(21) Appl. No.: 13/729,313

(22) Filed: Dec. 28, 2012

Related U.S. Application Data
(63) Continuation of application No. 12/984,373, filed on Jan. 4, 2011, now abandoned.
Application No. 12/984,373 is a continuation of application No. 11/780,625, filed on Jul. 20, 2007.

(51) Int. Cl. A61K 9/28; A61K 31/167; A61K 31/485

(52) U.S. Cl. CPC A61K 9/28; A61K 31/485; A61K 31/167
USPC 424/464; 514/282

(57) ABSTRACT

The preferred exemplary embodiments in the present application provide formulations and methods for the delivery of drugs, particularly drugs of abuse, having an abuse-relevant drug substantially confined in the core and a non-abuse relevant drug in a non-core region. These formulations have reduced potential for abuse. In the formulation, preferably the abuse relevant drug is an opioid and the non-abuse relevant drug is acetaminophen or ibuprofen. More preferably, the opioid is hydrocodone, and the non-abuse relevant analgesic is acetaminophen. In certain preferred embodiments, the dosage forms are characterized by resistance to solvent extraction; tampering, crushing or grinding. Certain embodiments of the inventions provide dosage forms that provide an initial burst of release of drug followed by a prolonged period of controllable drug release.

### TECHNICAL FIELD OF INVENTION

The present invention relates to compositions for oral administration. Preferably the invention teaches at least one abuse-resistant composition for delivering a drug having an abuse potential, related methods of preparing these dosage forms, and methods of treating a patient in need thereof comprising administering the inventive compositions to the patient. More preferably, these compositions include at least one non-opioid analgesic and at least one confined opioid analgesic.

### BACKGROUND OF THE INVENTION

Abuse of prescription drugs has become a public health problem in many communities. Opioids are one common class of drugs that is subject to abuse. Opioids are the major class of analgesics used in the management of moderate to severe pain in the United States of America because of their effectiveness, ease of titration, and favorable risk-to-benefit ratio.

One of the effects of opioid administration is the ability of such drugs in some individuals to alter mood and feeling in a manner so as to provide a desirable sense of “well-being” dissociated from therapeutic ameliorative effects. Repeated illicit abuse further results in certain users being addicted to opioids. Similar to the opioids, many other classes of drugs are also subject to abuse, although the patterns and effects of the abuse vary.

Accordingly, in the art various methods and formulations have been described to diminish or eliminate various patterns of abuse, such as related to accidental or intentional dose dumping in alcohol, crushing and snorting, etc.

U.S. patent application Ser. No. 11/625,705 and PCT Application PCT/US07/60864 filed on Jan. 22, 2007, which are incorporated herein by reference in their entirety for all purposes, describe various methods and compositions of abuse resistant formulations having drugs of abuse. In these patent applications, an extensive formulation screening program was used to identify suitable extrudate formulations exhibiting biphasic in vitro drug dissolution (>30% after 1 h, >80% after 8 h) for the narcotic drug hydrocodone bitartrate 2.5-hydrate. It was found, however, that the drug dissolution of the second agent did not meet the above criterion for biphasic drug dissolution (with >30% after 1 h, >80% after 8 h) with respect to acetaminophen, a.k.a. paracetamol or APAP. Although both drugs, hydrocodone-bitartrate 2.5-hydrate and acetaminophen, were extruded and calendered from a homogeneously blended mixture of solids, all the studies on the resulting dosage forms showed that the two active ingredients were released at different rates. These in vitro data were also confirmed in experimental animal studies (minipig) and in a clinical study performed with these dosage forms. The clinical study also showed that although the desired kinetics were achieved for the hydrocodone bitartrate 2.5-hydrate, this was not the case for the acetaminophen. New formulation concepts therefore had to be found to achieve the required biphasic drug dissolution profile for the acetaminophen as well. Further, it was also found that in most cases the calendered extrudate tablets manufactured in accordance with U.S. Ser. No. 11/625,705 and PCT/US07/60864 patent applications had rough surfaces and therefore based of their appearance did not in all cases meet the criteria for marketable tablets. A need for improvement was thus also perceived in this respect.

While numerous compositions, formulations and methodologies exist to address abuse of drugs, all compositions, formulations and methods have limitations to a greater or lesser extent. Accordingly, there is a need for providing new and/or improved formulations, compositions and methods of preventing abuse of drugs having abuse potential. More specifically, there is a need to develop oral formulations that would meet the biphasic drug dissolution profile and also have attributes that include drug deterrence and desirable appearance to meet the criteria for a marketable tablet.

This background information is provided for the purpose of making known some information believed by the applicant to be of possible relevance to the present invention. No admission is intended, nor should be construed, that any of the preceding information constitutes prior art to the present invention.

### SUMMARY OF THE INVENTION

Certain preferred embodiments of the present invention provide dosage forms and methods for the delivery of drugs, particularly drugs of abuse, characterized by resistance to solvent extraction; tampering, crushing or grinding, and providing an initial burst of release of drug followed by a prolonged period of controllable drug release. Preferably, the dosage form includes at least one non-opioid analgesic and at least one confined opioid analgesic.

In one preferred embodiment, the present invention provides a pharmaceutical composition having a core and a non-core layer, comprising: (a) hydrocodone, a pharmaceutically acceptable salt or a hydrate thereof, and (b) acetaminophen or ibuprofen. In this embodiment, at least 75% all of the hydrocodone, pharmaceutically acceptable salt or hydrate thereof is in the core, and the acetaminophen or the ibuprofen is the non-core layer. Further, this composition is adapted so as to be useful for oral administration to a human 3, 2, or 1 times daily. Preferably, greater than 90% of the hydrocodone, pharmaceutically acceptable salt or hydrate thereof is in the core. More preferably, substantially all of the hydrocodone, pharmaceutically acceptable salt or hydrate thereof is in the core. In another embodiment, the core further comprises acetaminophen or ibuprofen. More preferably, the core further comprises acetaminophen.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma profile characterized by a Cmaxfor hydrocodone from about 0.6 ng/mL/mg to about 1.4 ng/mL/mg and a Cmax for acetaminophen from about 2.8 ng/mL/mg and 7.9 ng/mL/mg after a single dose. In another embodiment, the pharmaceutical composition produces a plasma profile characterized by a Cmax for hydrocodone of about 0.4 ng/mL/mg to about 1.9 ng/mL/mg and a Cmax for acetaminophen of about 2.0 ng/mL/mg to about 10.4 ng/mL/mg after a single dose. In yet another embodiment, the pharmaceutical composition produces a plasma profile characterized by a Cmax for hydrocodone of from about 0.6 ng/mL/mg to about 1.0 ng/mL/mg and a Cmax for acetaminophen of from about 3.0 ng/mL/mg to about 5.2 ng/mL/mg after a single dose. Other embodiments of the dosage form include about 5-20 mg of hydrocodone bitartrate pentahemihydrate and about 400-600 mg of acetaminophen. Yet another embodiment of the dosage form includes 10-15 mg of hydrocodone bitartrate pentahemihydrate and about 500-600 mg of acetaminophen.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. When administered to the human patient, the dosage form produces an AUC for hydrocodone of about 9.1 ng*hr/mL/mg to about 19.9 ng*hr/mL/mg and an AUC for acetaminophen of about 28.6 ng*hr/mL/mg to about 59.1 ng*hr/mL/mg. In another embodiment, the dosage form produces an AUC for hydrocodone of about 7.0 ng*hr/mL/mg to about 26.2 ng*hr/mL/mg and an AUC for acetaminophen of about 18.4 ng*hr/mL/mg to about 79.9 ng*hr/mL/mg. In yet another embodiment, the dosage form produces an AUC for hydrocodone of about 11.3 ng*hr/mL/mg to about 18.7 ng*hr/mL/mg and an AUC for acetaminophen of about 28.7 ng*hr/mL/mg to about 53.5 ng*hr/mL/mg. Preferably in this embodiment, the in vitro rate of release of the pharmaceutical composition has a biphasic release profile, and wherein for each phase of the in vitro rate of release is zero order or first order for acetaminophen and zero order or first order for hydrocodone bitartrate pentahemihydrate.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. The dosage form produces a plasma concentration at 1 hour (Cl) for hydrocodone of about 0.18 ng/mL/mg to about 1.51 ng/mL/mg, and a plasma concentration at 1 hour Cl for acetaminophen of about 2.34 ng/mL/mg to about 7.24 ng/mL/mg. In preferred embodiments such as Formulation 15, the dosage form produces a Cl for hydrocodone of about 0.32 ng/mL/mg to about 1.51 ng/mL/mg and a Cl for acetaminophen of about 2.34 ng/mL/mg to about 5.50 ng/mL/mg.

In certain other embodiments, the following pharmacokinetic profile is preferably exhibited when the single dose comprises about 15 mg of hydrocodone bitartrate pentahemihydrate and about 500 mg of acetaminophen, administered to the patient, when fasting. The dosage form produces a plasma concentration at 1 hour (Cl) for hydrocodone from about 0.30 ng/mL/mg to about 1.06 ng/mL/mg, and a Cl for acetaminophen from about 2.75 ng/mL/mg to about 5.57 ng/mL/mg. In preferred embodiments, the dosage from produces a Cl for hydrocodone from about 0.45 ng/mL/mg to about 1.06 ng/mL/mg and a Cl for acetaminophen from about 2.75 ng/mL/mg to about 4.43 ng/mL/mg.

In other embodiments, the dosage form produces a combined Cl for hydrocodone and acetaminophen from about 1.18 μg/mL to about 3.63 μg/mL, after a single dose of 15 mg hydrocodone bitartrate pentahemihydrate and 500 mg of acetaminophen, on fasting. In preferred embodiments, the dosage from produces a combined Cl for hydrocodone and acetaminophen from about 1.18 μg/mL to about 2.76 μg/mL, after a single dose of 15 mg hydrocodone bitartrate pentahemihydrate and 500 mg of acetaminophen.

In certain embodiments, the dosage form produces a combined Cl for hydrocodone and acetaminophen from about 1.38 μg/mL to about 2.79 μg/mL, after a single dose of 15 mg hydrocone bitartrate pentahemihydrate and 500 mg of acetaminophen. In preferred embodiments, the dosage from produces a combined Cl for hydrocodone and acetaminophen from about 1.38 μg/mL to about 2.23 μg/mL, after a single dose of 15 mg hydrocodone bitartrate pentahemihydrate and 500 mg of acetaminophen.

In preferred embodiments, the dosage form produces a combined Cl for hydrocodone and acetaminophen of 1.80±0.42 μg/mL with the 95% confidence interval for the mean value falling between about 1.61 μg/mL to about 2.00 μg/mL, after a single dose of 15 mg hydrocodone bitartrate pentahemihydrate and 500 mg of acetaminophen. The 95% confidence interval of combined Cl for hydrocodone and acetaminophen for the preferred embodiments and the Control overlapped. The 95% confidence interval for the mean value of combined Cl for hydrocodone and acetaminophen for the Control ranged from about 1.46 to 1.96 μg/mL, after administered as a single dose of 15 mg hydrocodone and 500 mg of acetaminophen to the human patient. The Control provides sufficient plasma levels of opioid and nonopioid analgesic to provide a reduction in pain intensity within about 1 hour after administration.

When administered to a population of healthy North Americans or Western Europeans, particularly when the formulation is adapted to be suitable for, or intended for, administration to a human every 12 hours as needed, about 20-45% of the hydrocodone is released in vitro from the pharmaceutical compositions in about 1 hour and about 20-45% of the acetaminophen is released in vitro from the pharmaceutical compositions in about 1 hour in 0.01 N HCl at 50 rpm at 37° C. In another embodiment, about 25-35% of the hydrocodone is released in vitro from the pharmaceutical compositions in about 1 hour and about 25-35% of the acetaminophen is released in vitro from the pharmaceutical compositions in about 1 hour in 0.01 N HCl at 50 rpm at 37° C. Further, in another embodiment, at least 90% of the hydrocodone is released from the pharmaceutical composition in about 8 hours to about 12 hours and at least 60% to about 99% of the acetaminophen is released in vitro from the pharmaceutical compositions in about 6 hours to about 8.5 hours. In another embodiment, at least 90% of the hydrocodone is released from the pharmaceutical composition in about 8 hours to about 11 hours and at least 90% of the acetaminophen is released in vitro from the pharmaceutical compositions in about 8 hours to about 11 hours. In another embodiment, at least 95% of the hydrocodone is released from the pharmaceutical composition in about 9 hours to about 12 hours and at least 95% of the acetaminophen is released in vitro from the pharmaceutical compositions in about 9 hours to about 12 hours. Yet in another embodiment, at least 95% is of the hydrocodone is released from the pharmaceutical composition in about 10 hours to about 12 hours and at least 95% of the acetaminophen is released in vitro from the pharmaceutical compositions in about 10 hours to about 12 hours. In another embodiment, at least 99% of the hydrocodone is released from the pharmaceutical composition in about 11 hours to about 12 hours and at least 99% of the acetaminophen is released in vitro from the pharmaceutical compositions in about 11 hours to about 12 hours. In yet another embodiment, at least 99% of the hydrocodone is released from the pharmaceutical composition in less than about 13 hours and at least 99% of the acetaminophen is released in vitro from the pharmaceutical compositions in less than about 13 hours.

However, when the a slow-release version of the formulation is adapted to be suitable for, or intended for administration to a human, twice daily, as needed, then at least 90% of the hydrocodone is released from the pharmaceutical composition in about 18 hours to about 23 hours and at least 90% of the acetaminophen is released in vitro from the pharmaceutical compositions in about 18 hours to about 23 hours. In another embodiment of the slow release formulation, at least 95% of the hydrocodone is released from the pharmaceutical composition in about 20 hours to about 25 hours and at least 95% of the acetaminophen is released in vitro from the pharmaceutical compositions in about 20 hours to about 25 hours. In another embodiment of the slow release formulation, at least 95% is of the hydrocodone is released from the pharmaceutical composition in about 21 hours to about 22 hours and at least 95% of the acetaminophen is released in vitro from the pharmaceutical compositions in about 21 hours to about 22 hours. In another embodiment of this slow release embodiment, at least 99% of the hydrocodone is released from the pharmaceutical composition in about 22 hours to about 26 hours and at least 99% of the acetaminophen is released in vitro from the pharmaceutical compositions in about 22 hours to about 26 hours. In yet another embodiment of the slow release formulation, at least 99% of the hydrocodone is released from the pharmaceutical composition in less than about 27 hours and at least 99% of the acetaminophen is released in vitro from the pharmaceutical compositions in less than about 27 hours.

In a preferred embodiment, the present invention provides a composition where the core layer comprises an excipient or a mixture of excipients capable of controlling the drug release and the non-core layer comprises an excipient capable of instantly releasing the drug. Further, in a preferred embodiment, the core layer is manufactured by melt-extrusion followed by direct shaping of the drug-containing melt and the non-core layer is spray coated over the core layer. Most preferably, the composition comprises about 500 mg of acetaminophen and about 15 mg of hydrocodone bitartrate pentahemihydrate.

In another exemplary embodiment, the present invention provides a pharmaceutical composition having a core and a non-core layer, comprising: (a) an abuse-relevant drug, a pharmaceutically acceptable salt or a hydrate thereof and a non-abuse-relevant drug or a pharmaceutically acceptable salt thereof in the core layer, and (b) a non-abuse-relevant drug, a pharmaceutically acceptable salt or a hydrate thereof in the non-core layer. Preferably, this composition is characterized by at least one of the following features:

i) the amount of abuse-relevant drug that is extracted from the composition by 40% aqueous ethanol within one hour at 37° C. in vitro is less than or equal 1.5 times the amount of the abuse-relevant drug that is extracted by 0.01 N hydrochloric acid in vitro within one hour at 37° C.,
ii) the composition does not break under a force of 150 newtons, preferably 300 newtons, more preferably 450 newtons, yet more preferably 500 newtons as measured by “Pharma Test PTB 501” hardness tester,
iii) the composition releases at least 20% of the abuse-relevant drug and not more than 45% of the abuse-relevant drug during the first hour of in vitro dissolution testing and preferably also during the first hour of in vivo testing,
iv) the composition releases a therapeutically effective dose of the non-abuse relevant drug within 1 to 2 hours after a single dose,
v) the composition releases a therapeutically effective dose of the non-abuse relevant drug and/or the abuse-relevant drug at 1 hour and at 12 hours after a single dose,
vi) in the composition, release of the abuse-relevant drug upon grinding increases by less than 2- to 3-fold, as compared to an intact tablet, when the composition is ground for 1 minute by a coffee-grinder at 20,000-50,000 rpm, in 40% aqueous ethanol for 1 hour at 37° C.,
vii) the composition when ground comprises a particulate size of about 2 cm to about 355 micrometer for about 20% of the fraction, greater than about 63 microns and less than about 355 microns for about 66% of the fraction and less than about 63 microns for about 14% of the fraction, as measured by a sieving test, or
viii) the composition is substantially smooth, wherein the Centre Line Average (CLA) is from about 0.1 to about 0.6, preferably from about 0.1 to about 0.4, and most preferably from about 0.1 to about 0.2.

In this composition, the amount of the abuse-relevant drug that is extracted from the formulation by 40% aqueous ethanol within one hour at 37° C. is about 70% to about 130% of the amount of the drug that is extracted by 0.01 N hydrochloric acid within one hour at 37° C. In another embodiment, the amount of the abuse-relevant drug that is extracted from the formulation by 40% aqueous ethanol within one hour at 37° C. is about 70% to about 90% of the amount of the drug that is extracted by 0.01 N hydrochloric acid within one hour at 37° C. In yet another embodiment, the abuse-relevant drug that is extracted from the formulation by 40% aqueous ethanol within one hour at 37° C. is about 75% to about 90% of the amount of the drug that is extracted by 0.01 N hydrochloric acid within one hour at 37° C.

Another embodiment of the present invention provides a pharmaceutical composition having a core layer and a non-core layer. In this composition the core layer comprises a mixture of: (a) at least one opioid; and (b) at least one rate altering pharmaceutically acceptable polymer, copolymer, or a combination thereof. The non-core layer comprises at least one non-opioid analgesic. Further, these compositions are adapted so as to be useful for oral administration to a human 3, 2, or 1 times daily. Preferably, the core layer further comprises at least one non-opioid analgesic. In a preferred embodiment, the composition is characterized by at least one of the following features:

i) the amount of abuse-relevant drug that is extracted from the composition by 40% aqueous ethanol within one hour at 37° C. in vitro is less than or equal 1.5 times the amount of the abuse-relevant drug that is extracted by 0.01 N hydrochloric acid in vitro within one hour at 37° C.,
ii) the composition does not break under a force of 150 newtons, preferably 300 newtons, more preferably 450 newtons, yet more preferably 500 newtons as measured by “Pharma Test PTB 501” hardness tester,
iii) the composition releases at least 20% of the abuse-relevant drug and not more than 45% of the abuse-relevant drug during the first hour of in vitro dissolution testing and preferably also during the first hour of in vivo testing,
iv) the composition releases a therapeutically effective dose of the non-abuse relevant drug within 1 to 2 hours after a single dose,
v) the composition releases a therapeutically effective dose of the non-abuse relevant drug and/or the abuse-relevant drug at 1 hour and at 12 hours after a single dose,
vi) in the composition, release of the abuse-relevant drug upon grinding increases by less than 2- to 3-fold, as compared to an intact tablet, when the composition is ground for 1 minute by a coffee-grinder at 20,000-50,000 rpm, in 40% aqueous ethanol for 1 hour at 37° C.,
vii) the composition when ground comprises a particulate size of about 2 cm to about 355 micrometer for about 20% of the fraction, greater than about 63 microns and less than about 355 microns for about 66% of the fraction and less than about 63 microns for about 14% of the fraction, as measured by a sieving test, or
viii) the composition is substantially smooth, wherein the Centre Line Average (CLA) is from about 0.1 to about 0.6, preferably from about 0.1 to about 0.4, and most preferably from about 0.1 to about 0.2.

In one embodiment, the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvreturn, pentazocine, phenadoxone, phenazocine, phenomorphan, phenoperidine, piminodine, propiram, propoxyphene, sufentanil, tilidine, and tramadol, and salts, hydrates and mixtures thereof. Further, the non-opioid analgesic is selected from the group consisting of acetaminophen, aspirin, fentaynl, ibuprofen, indomethacin, ketorolac, naproxen, phenacetin, piroxicam, sufentanyl, sunlindac, interferon alpha, and salts, hydrates and mixtures thereof. Preferably, the opioid is hydrocodone and the non-opioid analgesic is acetaminophen or ibuprofen. More preferably, the opioid is hydrocodone and the non-opioid analgesic is acetaminophen.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma profile characterized by a Cmax for hydrocodone from about 0.6 ng/mL/mg to about 1.4 ng/mL/mg and a Cmax for acetaminophen from about 2.8 ng/mL/mg and 7.9 ng/mL/mg after a single dose. In another embodiment, the pharmaceutical composition produces a plasma profile characterized by a Cmax for hydrocodone of about 0.4 ng/mL/mg to about 1.9 ng/mL/mg and a Cmax for acetaminophen of about 2.0 ng/mL/mg to about 10.4 ng/mL/mg after a single dose. In yet another embodiment, the pharmaceutical composition produces a plasma profile characterized by a Cmax for hydrocodone of from about 0.6 ng/mL/mg to about 1.0 ng/mL/mg and a Cmax for acetaminophen of from about 3.0 ng/mL/mg to about 5.2 ng/mL/mg after a single dose.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. When administered to the human patient, the dosage form produces an AUC for hydrocodone of about 9.1 ng*hr/mL/mg to about 19.9 ng*hr/mL/mg and an AUC for acetaminophen of about 28.6 ng*hr/mL/mg to about 59.1 ng*hr/mL/mg. In another embodiment, the dosage form produces an AUC for hydrocodone of about 7.0 ng*hr/mL/mg to about 26.2 ng*hr/mL/mg and an AUC for acetaminophen of about 18.4 ng*hr/mL/mg to about 79.9 ng*hr/mL/mg. In yet another embodiment, the dosage form produces an AUC for hydrocodone of about 11.3 ng*hr/mL/mg to about 18.7 ng*hr/mL/mg and an AUC for acetaminophen of about 28.7 ng*hr/mL/mg to about 53.5 ng*hr/mL/mg. Preferably in this embodiment, the in vitro rate of release of the pharmaceutical composition has a biphasic release profile, and wherein for each phase of the in vitro rate of release is zero order or first order for acetaminophen and zero order or first order for hydrocodone bitartrate pentahemihydrate.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone of about 0.18 ng/mL/mg to about 1.51 ng/mL/mg, and a plasma concentration at 1 hour Cl for acetaminophen of about 2.34 ng/mL/mg to about 7.24 ng/mL/mg. In preferred embodiments such as Formulation 15, the dosage form produces a Cl for hydrocodone of about 0.32 ng/mL/mg to about 1.51 ng/mL/mg and a Cl for acetaminophen of about 2.34 ng/mL/mg to about 5.50 ng/mL/mg.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone from about 0.30 ng/mL/mg to about 1.06 ng/mL/mg, and a Cl for acetaminophen from about 2.75 ng/mL/mg to about 5.57 ng/mL/mg. In preferred embodiments, the dosage from produces a Cl for hydrocodone from about 0.45 ng/mL/mg to about 1.06 ng/mL/mg and a Cl for acetaminophen from about 2.75 ng/mL/mg to about 4.43 ng/mL/mg.

In certain embodiments, the dosage form produces a combined Cl for hydrocodone and acetaminophen from about 1.18 μg/mL to about 3.63 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.18 μg/mL to about 2.76 μg/mL, after a single dose of 15 mg hydrocodone bitartrate pentahemihydrate and 500 mg of acetaminophen.

In certain embodiments, the dosage form produces a combined Cl for hydrocodone and acetaminophen from about 1.38 μg/mL to about 2.79 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.38 μg/mL to about 2.23 μg/mL, after a single dose of 15 mg hydrocodone bitartrate pentahemihydrate and 500 mg of acetaminophen.

In preferred embodiments, the dosage form produces a combined Cl for hydrocodone and acetaminophen of 1.80±0.42 μg/mL with the 95% confidence interval for the mean value falling between about 1.61 μg/mL to about 2.00 μg/mL, after a single dose of 15 mg hydrocodone bitartrate pentahemihydrate and 500 mg of acetaminophen. The 95% confidence interval of combined Cl for hydrocodone and acetaminophen for the preferred embodiments and the Control overlapped. The 95% confidence interval for the mean value of combined Cl for hydrocodone and acetaminophen for the Control ranged from about 1.46 to 1.96 μg/mL, after administered as a single dose of 15 mg hydrocodone bitartrate pentahemihydrate and 500 mg of acetaminophen to the human patient. The Control provides sufficient plasma levels of opioid and nonopioid analgesic to provide a reduction in pain intensity within about 1 hour after administration.

When administered to a population of healthy North Americans or Western Europeans, particularly when the formulation is adapted to be suitable for, or intended for, administration to a human every 12 hours as needed, about 20-45% of the hydrocodone is released in vitro from the pharmaceutical compositions in about 1 hour and about 20-45% of the acetaminophen is released in vitro from the pharmaceutical compositions in about 1 hour in 0.01 N HCl at 50 rpm at 37° C. In another embodiment, about 25-35% of the hydrocodone is released in vitro from the pharmaceutical compositions in about 1 hour and about 25-35% of the acetaminophen is released in vitro from the pharmaceutical compositions in about 1 hour in 0.01 N HCl at 50 rpm at 37° C. Further, in another embodiment, at least 90% of the hydrocodone is released from the pharmaceutical composition in about 8 hours to about 12 hours and at least 60% to about 99% of the acetaminophen is released in vitro from the pharmaceutical compositions in about 6 hours to about 8.5 hours. In another embodiment, at least 90% of the hydrocodone is released from the pharmaceutical composition in about 8 hours to about 11 hours and at least 90% of the acetaminophen is released in vitro from the pharmaceutical compositions in about 8 hours to about 11 hours. In another embodiment, at least 95% of the hydrocodone is released from the pharmaceutical composition in about 9 hours to about 12 hours and at least 95% of the acetaminophen is released in vitro from the pharmaceutical compositions in about 9 hours to about 12 hours. Yet in another embodiment, at least 95% is of the hydrocodone is released from the pharmaceutical composition in about 10 hours to about 12 hours and at least 95% of the acetaminophen is released in vitro from the pharmaceutical compositions in about 10 hours to about 12 hours. In another embodiment, at least 99% of the hydrocodone is released from the pharmaceutical composition in about 11 hours to about 12 hours and at least 99% of the acetaminophen is released in vitro from the pharmaceutical compositions in about 11 hours to about 12 hours. In yet another embodiment, at least 99% of the hydrocodone is released from the pharmaceutical composition in less than about 13 hours and at least 99% of the acetaminophen is released in vitro from the pharmaceutical compositions in less than about 13 hours.

However, when the a slow-release version of the formulation is adapted to be suitable for, or intended for administration to a human, twice daily, as needed, then at least 90% of the hydrocodone is released from the pharmaceutical composition in about 18 hours to about 23 hours and at least 90% of the acetaminophen is released in vitro from the pharmaceutical compositions in about 18 hours to about 23 hours. In another embodiment of the slow release formulation, at least 95% of the hydrocodone is released from the pharmaceutical composition in about 20 hours to about 25 hours and at least 95% of the acetaminophen is released in vitro from the pharmaceutical compositions in about 20 hours to about 25 hours. In another embodiment of the slow release formulation, at least 95% is of the hydrocodone is released from the pharmaceutical composition in about 21 hours to about 22 hours and at least 95% of the acetaminophen is released in vitro from the pharmaceutical compositions in about 21 hours to about 22 hours. In another embodiment of this slow release embodiment, at least 99% of the hydrocodone is released from the pharmaceutical composition in about 22 hours to about 26 hours and at least 99% of the acetaminophen is released in vitro from the pharmaceutical compositions in about 22 hours to about 26 hours. In yet another embodiment of the slow release formulation, at least 99% of the hydrocodone is released from the pharmaceutical composition in less than about 27 hours and at least 99% of the acetaminophen is released in vitro from the pharmaceutical compositions in less than about 27 hours.

In a preferred embodiment, the present invention provides a composition where the core layer comprises an excipient capable of controlling the drug release and the non-core layer comprises an excipient capable of instantly releasing the drug. Further, in a preferred embodiment, the core layer is manufactured by melt-extrusion followed by direct shaping of the drug-containing melt and the non-core layer is spray coated over the core layer. Most preferably, the composition comprises about 500 mg of acetaminophen and about 15 mg of hydrocodone bitartrate pentahemihydrate.

In another embodiment, the present invention provides a pharmaceutical composition having a core layer and a non-core layer. In this composition, the core layer comprises a mixture of (a) at least one opioid and at least one first non-opioid analgesic; (b) at least one rate altering pharmaceutically acceptable polymer, copolymer, or a combination thereof. The non-core layer comprises at least one second non-opioid analgesic. Further, the composition is adapted so as to be useful for oral administration to a human 3, 2, or 1 times daily. In this embodiment, preferably, the opioid comprises hydrocodone and the first and the second non-opioid analgesic comprises acetaminophen or ibuprofen. More preferably, the opioid comprises hydrocodone and the first and the second non-opioid analgesic comprises acetaminophen. Further, in this embodiment, the non-core layer comprises: (a) acetaminophen; and (b) at least one rate altering pharmaceutically acceptable polymer, copolymer, or a combination thereof. Preferably, the polymer or copolymer is selected from the group consisting of: hydroxypropyl cellulose, hydroxypropyl methylcellulose, hydroxyethyl cellulose; polymethacrylate, polyvinyl alcohol, polyethylene oxide, and combinations thereof. More preferably, the polymer or copolymer is selected from the group consisting of: hydroxypropyl methylcellulose, and polyvinyl alcohol, or combinations thereof. Yet more preferably, the polymer or copolymer is selected from the group consisting of: polyvinyl alcohol and polyethylene oxide graft copolymers. Further, in this embodiment, the ratio of acetaminophen to the rate controlling polymer or copolymer or combination thereof is about 1:1 to about 10:1. More preferably, the ratio of acetaminophen to the rate controlling polymer or copolymer or combination thereof is about 3:1 to about 5:1. As provided in the present invention, in one preferred embodiment,

the non-core layer has at least one of the following characteristics:
(a) substantially does not crack after 3 months at 40° C., 75% relative humidity in induction-sealed HDPE bottles;
(b) substantially dry (not sticky);
provides fast dissolution in 0.01N HCl at 37° C. to expose the core layer
releases at least 80% of the acetaminophen in the non-core layer within 20 minutes of administration to a human patient; or
(e) provides a white pigmentation to the formulation without additional pigments.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma profile characterized by a Cmax for hydrocodone from about 0.6 ng/mL/mg to about 1.4 ng/mL/mg and a Cmax for acetaminophen from about 2.8 ng/mL/mg and 7.9 ng/mL/mg after a single dose. In another embodiment, the pharmaceutical composition produces a plasma profile characterized by a Cmax for hydrocodone of about 0.4 ng/mL/mg to about 1.9 ng/mL/mg and a Cmax for acetaminophen of about 2.0 ng/mL/mg to about 10.4 ng/mL/mg after a single dose. In yet another embodiment, the pharmaceutical composition produces a plasma profile characterized by a Cmax for hydrocodone of from about 0.6 ng/mL/mg to about 1.0 ng/mL/mg and a Cmax for acetaminophen of from about 3.0 ng/mL/mg to about 5.2 ng/mL/mg after a single dose.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. When administered to the human patient, the dosage form produces an AUC for hydrocodone of about 9.1 ng*hr/mL/mg to about 19.9 ng*hr/mL/mg and an AUC for acetaminophen of about 28.6 ng*hr/mL/mg to about 59.1 ng*hr/mL/mg. In another embodiment, the dosage form produces an AUC for hydrocodone of about 7.0 ng*hr/mL/mg to about 26.2 ng*hr/mL/mg and an AUC for acetaminophen of about 18.4 ng*hr/mL/mg to about 79.9 ng*hr/mL/mg. In yet another embodiment, the dosage form produces an AUC for hydrocodone of about 11.3 ng*hr/mL/mg to about 18.7 ng*hr/mL/mg and an AUC for acetaminophen of about 28.7 ng*hr/mL/mg to about 53.5 ng*hr/mL/mg. Preferably in this embodiment, the in vitro rate of release of the pharmaceutical composition has a biphasic release profile, and wherein for each phase of the in vitro rate of release is zero order or first order for acetaminophen and zero order or first order for hydrocodone bitartrate pentahemihydrate.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone of about 0.18 ng/mL/mg to about 1.51 ng/mL/mg, and a plasma concentration at 1 hour Cl for acetaminophen of about 2.34 ng/mL/mg to about 7.24 ng/mL/mg. In preferred embodiments such as Formulation 15, the dosage form produces a Cl for hydrocodone of about 0.32 ng/mL/mg to about 1.51 ng/mL/mg and a Cl for acetaminophen of about 2.34 ng/mL/mg to about 5.50 ng/mL/mg.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone from about 0.30 ng/mL/mg to about 1.06 ng/mL/mg, and a Cl for acetaminophen from about 2.75 ng/mL/mg to about 5.57 ng/mL/mg. In preferred embodiments, the dosage from produces a Cl for hydrocodone from about 0.45 ng/mL/mg to about 1.06 ng/mL/mg and a Cl for acetaminophen from about 2.75 ng/mL/mg to about 4.43 ng/mL/mg. In certain embodiments, the dosage form produces a combined Cl for hydrocodone and acetaminophen from about 1.18 μg/mL to about 3.63 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.18 μg/mL to about 2.76 μg/mL, after a single dose of 15 mg hydrocodone bitartrate pentahemihydrate and 500 mg of acetaminophen.

In certain embodiments, the dosage form produces a combined Cl for hydrocodone and acetaminophen from about 1.38 μg/mL to about 2.79 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.38 μg/mL to about 2.23 μg/mL, after a single dose of 15 mg hydrocodone bitartrate pentahemihydrate and 500 mg of acetaminophen.

In preferred embodiments, the dosage form produces a combined Cl for hydrocodone and acetaminophen of 1.80±0.42 μg/mL with the 95% confidence interval for the mean value falling between about 1.61 μg/mL to about 2.00 μg/mL, after a single dose of 15 mg hydrocodone bitartrate pentahemihydrate and 500 mg of acetaminophen. The 95% confidence interval of combined Cl for hydrocodone and acetaminophen for the preferred embodiments and the Control overlapped. The 95% confidence interval for the mean value of combined Cl for hydrocodone and acetaminophen for the Control ranged from about 1.46 to 1.96 μg/mL, after administered as a single dose of 15 mg hydrocodone and 500 mg of acetaminophen to the human patient. The Control provides sufficient plasma levels of opioid and nonopioid analgesic to provide a reduction in pain intensity within about 1 hour after administration.

When administered to a population of healthy North Americans or Western Europeans, particularly when the formulation is adapted to be suitable for, or intended for, administration to a human every 12 hours as needed, about 20-45% of the hydrocodone is released in vitro from the pharmaceutical compositions in about 1 hour and about 20-45% of the acetaminophen is released in vitro from the pharmaceutical compositions in about 1 hour in 0.01 N HCl at 50 rpm at 37° C. In another embodiment, about 25-35% of the hydrocodone is released in vitro from the pharmaceutical compositions in about 1 hour and about 25-35% of the acetaminophen is released in vitro from the pharmaceutical compositions in about 1 hour in 0.01 N HCl at 50 rpm at 37° C. Further, in another embodiment, at least 90% of the hydrocodone is released from the pharmaceutical composition in about 8 hours to about 12 hours and at least 60% to about 99% of the acetaminophen is released in vitro from the pharmaceutical compositions in about 6 hours to about 8.5 hours. In another embodiment, at least 90% of the hydrocodone is released from the pharmaceutical composition in about 8 hours to about 11 hours and at least 90% of the acetaminophen is released in vitro from the pharmaceutical compositions in about 8 hours to about 11 hours. In another embodiment, at least 95% of the hydrocodone is released from the pharmaceutical composition in about 9 hours to about 12 hours and at least 95% of the acetaminophen is released in vitro from the pharmaceutical compositions in about 9 hours to about 12 hours. Yet in another embodiment, at least 95% is of the hydrocodone is released from the pharmaceutical composition in about 10 hours to about 12 hours and at least 95% of the acetaminophen is released in vitro from the pharmaceutical compositions in about 10 hours to about 12 hours. In another embodiment, at least 99% of the hydrocodone is released from the pharmaceutical composition in about 11 hours to about 12 hours and at least 99% of the acetaminophen is released in vitro from the pharmaceutical compositions in about 11 hours to about 12 hours. In yet another embodiment, at least 99% of the hydrocodone is released from the pharmaceutical composition in less than about 13 hours and at least 99% of the acetaminophen is released in vitro from the pharmaceutical compositions in less than about 13 hours.

However, when the a slow-release version of the formulation is adapted to be suitable for, or intended for administration to a human, twice daily, as needed, then at least 90% of the hydrocodone is released from the pharmaceutical composition in about 18 hours to about 23 hours and at least 90% of the acetaminophen is released in vitro from the pharmaceutical compositions in about 18 hours to about 23 hours. In another embodiment of the slow release formulation, at least 95% of the hydrocodone is released from the pharmaceutical composition in about 20 hours to about 25 hours and at least 95% of the acetaminophen is released in vitro from the pharmaceutical compositions in about 20 hours to about 25 hours. In another embodiment of the slow release formulation, at least 95% is of the hydrocodone is released from the pharmaceutical composition in about 21 hours to about 22 hours and at least 95% of the acetaminophen is released in vitro from the pharmaceutical compositions in about 21 hours to about 22 hours. In another embodiment of this slow release embodiment, at least 99% of the hydrocodone is released from the pharmaceutical composition in about 22 hours to about 26 hours and at least 99% of the acetaminophen is released in vitro from the pharmaceutical compositions in about 22 hours to about 26 hours. In yet another embodiment of the slow release formulation, at least 99% of the hydrocodone is released from the pharmaceutical composition in less than about 27 hours and at least 99% of the acetaminophen is released in vitro from the pharmaceutical compositions in less than about 27 hours.

In a preferred embodiment, the present invention provides a composition where the core layer comprises an excipient capable of controlling the drug release and the non-core layer comprises an excipient capable of instantly releasing the drug. Further, in a preferred embodiment, the core layer is manufactured by melt-extrusion followed by direct shaping of the drug-containing melt and the non-core layer is spray coated over the core layer. Most preferably, the composition comprises about 500 mg of acetaminophen and about 15 mg of hydrocodone bitartrate pentahemihydrate.

These and other objects, advantages, and features of the invention will become apparent to those persons skilled in the art upon reading the details of the methods of the invention and compositions used therein as more fully described below.

### BRIEF DESCRIPTION OF FIGURES

FIG. 1 depicts that coating the extrudated tablets resulted in significant smoothing of the tablet surface.

FIG. 2 depicts schematics for calculation of Surface Roughness using Centre Line Average (CLA) approach.

FIG. 3 depicts Centre Line Average (CLA) for an uncoated formulation. For uncoated formulation CLA=36.1, when (N=69).

FIG. 4 depicts Centre Line Average (CLA) for an uncoated formulation. For a coated formulation CLA=10.4, when (N=69).

FIG. 5A depicts preliminary mean hydrocodone concentration-time profiles for Formulations 15, and 16 and Control 1 for 48 hours.

FIG. 5B depicts preliminary mean hydrocodone concentration-time profiles for Formulations 15, and 16 and Control 1 for 12 hours.

FIG. 6A depicts preliminary mean acetaminophen concentration-time profiles for Formulations 15, and 16 and Control 1 for 48 hours.

FIG. 6B depicts preliminary mean acetaminophen concentration-time profiles for Formulations 15, and 16 and Control 1 for 12 hours.

FIG. 7A and FIG. 7B depict in vitro drug release profiles for hydrocodone and acetaminophen for Formulations 17, and 18, Control 2 and uncoated Formulation VM-1 for 480 minutes.

### DETAILED DESCRIPTION OF THE INVENTION

The invention is not limited to the particular methodology, protocols, animal studies, and reagents described, which can vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “an”, and “the” include plural reference unless the context clearly dictates otherwise. Thus, for example, reference to “a compound” includes a plurality of such compounds and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, animals, instruments, statistical analysis and methodologies which are reported in the publications which might be used in connection with the invention. Nothing herein is to be construed as an admission that the invention is not entitled to antedate such disclosure by virtue of prior invention.

Trademarks are used in this description as a convenient abbreviation for well known materials. As one of ordinary skill would appreciate, the following brand names indicate the substances indicated:

EUDRAGIT®: Polymers derived from esters of acrylic and methacrylic acid;
METHOCEL®: Methyl or methoxyl Cellulose
KOLLICOAT IR®: Polyvinyl alcohol-polyethylene glycol-graft copolymers
PLASDONE®: Polyvinylpyrrolidone polymer or -copolymer
LAUROGLYCOL®: Propylene glycol laurate ester
SPAN®: Sorbitan fatty acid esters

### CREMOPHOR®: Polyethoxylated Castor oil

POLOXAMER®: Polyoxyethylene polyoxypropylene block copolymers or polyoxyethylene polypropyleneglycol
TWEEN®: Polyethoxylated Sorbitan esters

### KLUCEL®: Hydroxypropylcellulose

KOLLIDON®: Polyvinlypyrrolidone homo- or copolymers
XYLITOL®: (2,3,4,5)tetrahydroxy-pentanol
ISOMALT®: An equimolar composition of 6-0-α-D-glucopyranosido-D-sorbitol (1,6-GPS) and 1-0-α-D-glucopyranosido-D-mannitol-dihydrate (1,1-GPM-dihydrate).
POLYOX®: Water-Soluble Resins based on polyethyleneoxide
XYLIT®: (2,3,4,5)tetrahydroxy-pentanol
PLUROL OLEIQUE®: Oleic esters of polyglycerol
LUTROL®: Polyoxyethylene polyoxypropylene block copolymers or polyoxyethylene polypropyleneglycol

### ETHOCEL®: Ethylcellulose

PRIMOJEL®: Sodium starch glycolate

The present invention provides an improved solid or solid solution, oral dosage formulation that provides for the in vivo sustained-release of pharmaceutically active compounds (“drugs”) that have properties that make them likely to be abused or have been shown to be frequently abused, as well as salts, esters, prodrugs and other pharmaceutically-acceptable equivalents thereof.

The term “AUC” refers to the area under the concentration time curve, calculated using the trapezoidal rule and Clast/k, where Clast is the last observed concentration and k is the calculated elimination rate constant.

The term “AUCt” refers to the area under the concentration time curve to last observed concentration calculated using the trapezoidal rule.

The term “Cmax” refers to the plasma concentration of the referent abuse relevant drug at Tₘₐₓ, expressed as ng/mL and μg/mL, respectively, produced by the oral ingestion of a composition of the invention. Unless specifically indicated, Cmax refers to the overall maximum observed concentration.

The term “Cmin” refers to the minimum observed concentration within the intended dosing interval, e.g., a twelve hour dosing interval for a formulation labelled as suitable for dosing every 12 hours or as needed, of a dosage form of the invention administered for 5 doses contiguous dosing intervals.

The term “ng*hr/mL/mg” refers to the amount of the substance measured in nanograms times the number of hours per milliliter of blood divided by the milligrams of the abuse relevant drug administered to the animal or human.

As used herein, the phrase “ascending release rate” refers to a dissolution rate that generally increases over time, such that the drug dissolves in the fluid at the environment of use at a rate that generally increases with time, rather than remaining constant or decreasing, until the dosage form is depleted of about 80% of the drug.

When used in the above or other treatments, a therapeutically effective dose of one of the compounds of the present invention can be employed in pure form or, where such forms exist, in pharmaceutically acceptable salt, ester or prodrug form. The phrase “therapeutically effective dose” of the compound includes of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In one preferred embodiment, the invention provides dosage forms that inhibit the extraction of the drug by common solvents, e.g., without limitation, distilled aqueous ethanol, from the formulation. The formulation dissuades abuse by limiting the ability of persons to extract the opioid from the formulation (either intentionally or unintentionally), such that the opioid cannot easily be concentrated for parenteral administration. Also these abuse resistant formulations may not be easily broken down into smaller particulates or powder-form that are easily abused by nasal snorting. Such an abuse-resistant formulation does not require incorporation of an opioid antagonist (albeit, an opioid antagonist may be added to the preparation to further dissuade abuse). While not desiring to be bound by any particular theory, it is believed that incorporation of alkylcelluloses, such as (without limitation) hydroxymethylcelluloses, and preferably hydroxypropylmethylcelluloses contribute to the formulation's resistance to extraction in alcohol, particularly in 20% or 40% aqueous ethanol. The alkylcellulose preferably has at least 12% substitution with an alkylsubstituent, more preferably at least 16% substitution with an alkyl substituent, and most preferably at least 19% substitution with an alkyl substituent. Alkyl substitutions of the cellulose below about 40%, and more preferably below about 30%, are preferred in the context of the invention. Additionally, the alkyl substituent is preferably C₁-C₆, more preferably C₁, C₂ or C₄, and most preferably C₃, and can be straight-chained or branched when the alkyl substituent contains 3 or more carbon atoms.

In another preferred embodiment, the dosage forms optionally resists cutting, grinding, pulverization and the like. A convenient measure for this aspect of the invention is “breaking strength,” as measured by “Pharma Test PTB 501” hardness tester. The inventive formulation preferably has a breaking strength of at least 150 newtons (150 N). More preferably, the inventive formulation has breaking strength of at least 300 N, yet more preferably of at least 450 N, and yet more preferably of at least 500 N.

Breaking strength according to the present invention can be determined with a tablet 10 mm in diameter and 5 mm in width according to the method for determining the breaking strength of tablets published in the European Pharmacopoeia 1997, page 143, 144, method no. 2.9.8. A preferred apparatus used to measure breaking strength is a “Zwick Z 2.5” materials tester, Fmax=2.5 kN, draw max. 1150 mm with the set up comprising a column and a spindle, clearance behind of 100 mm, and a test speed of 0.1800 mm/min. Measurement can be performed using a pressure piston with screw-in inserts and a cylinder (10 mm diameter), a force transducer, (Fmax. 1 kN, diameter=8 mm, class 0.5 from 10 N, class 1 from 2 N to ISO 7500-1, Zwick gross force Fmax=1.45 kN). The apparatus can optionally be obtained from Zwick GmbH & Co. KG, Ulm, Germany.

Any suitable means can be used to produce the inventive composition. In a preferred embodiment, the formulation is preferably melt-processed, and more preferably melt-extruded, and then in either case directly shaped without milling or grinding the formulation. Notwithstanding the foregoing, it is contemplated that the directly shaped tablets of the formulation can be optionally coated with a swallowing aid, such as without limitation, a gelatin coat. While not desiring to be bound by any particular theory, it is believed that direct shaping to prevent undesirable sharp features from forming on the formulation without an intermediate grinding step contributes to the superior breaking strength of the formulation. Additionally, embodiments of the inventive formulation optionally gain additional breaking strength by employing at least two melt-processed polymers. While not ascribing to any particular theory, it is believed that the second melt-processed polymer preferentially interacts with the first melt-processed polymer so as to advantageously adjust the transition glass temperature of the composition as a whole during the formation of the tablet.

In one embodiment, the formulation may use a polymer, or a copolymer, or a combination thereof to create the melt-processed, and more preferably melt-extruded, directly shaped formulation. Polymers that are pharmacologically inactive and provide enteric coatings or sustained release profile for the formulation can also be used. In one embodiment, suitable polymers/copolymers include poly(meth)acrylate like e.g. Eudragit L- or S-type, which are pharmacologically inactive.

EUDRAGIT® is a tradename for some preferred polymers that are suitable for use in the invention and are derived from esters of acrylic and methacrylic acid. The properties of the EUDRAGIT polymers are principally determined by functional groups incorporated into the monomers of the EUDRAGIT polymers. The individual EUDRAGIT® grades differ in their proportion of neutral, alkaline or acid groups and thus in terms of physicochemical properties. Ammonioalklyl methacrylate copolymers or methacrylate copolymers may be used having the following formula:

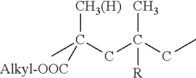

The Eudragit polymers fulfil the specifications/requirements set in the USP. According to 2007 US Pharmacopoeia, Eudragit is defined as USP 30/NF 25.

Methacrylic acid copolymer, type A NF=Eudragit L-100
Methacrylic acid copolymer, type B NF=Eudragit S-100
Methacrylic acid copolymer, type C NF=Eudragit L-100-55 (contains a small detergent amount)
Ammonio Methacrylate Copolymer, type A NF=Eudragit RL-100 (granules)
Ammonio Methacrylate Copolymer, type A NF=Eudragit RL-PO (powder)
Ammonio Methacrylate Copolymer, type B NF=Eudragit RS-100 (granules)
Ammonio Methacrylate Copolymer, type B NF=Eudragit RS-PO (powder)
Polyacrylate Dispersion 30 Percent Ph. Eur.=Eudragit NE30D (=30% aqueous dispersion)
Basic butylated methacrylate copolymer Ph. Eur.=Eudragit E-100
wherein the functional group has a quaternary ammonium (trimethylammonioethyl methacrylate) moiety or R═COOCH₂CH₂N⁺(CH₃)3Cl⁻ [commercially available as EUDRAGIT® (RL or RS)] or the functional group is a carboxylic acid, or R═COOH [commercially available as EUDRAGIT® (L)]. When the functional group is a carboxylic acid moiety, the EUDRAGIT® (L) polymer is gastroresistant and enterosoluble. Thus formulations using EUDRAGIT® (L) will be resistant to gastric fluid and will release the active agent in the colon. When the functional group is a trimethylammonioethyl methacrylate moiety, the EUDRAGIT® (RL or RS) polymers are insoluble, permeable, dispersible and pH-independent. These EUDRAGIT® (RL or RS) polymers may therefore be used for delayed drug release for sustained release formulations. EUDRAGIT® is sold in various forms such as in solid form (EUDRAGIT® L100/S100/L-100-55, EUDRAGIT® E PO, EUDRAGIT® RL PO, Eudragit RS PO), granules (EUDRAGIT® E100, EUDRAGIT®RL 100/RS 100), dispersions (L 30 D-55/FS 30D 30%, EUDRAGIT® NE 30 D/40 D 30%/40% polymer content, EUDRAGIT®RL 30 D RS 30 D 30%) and organic solutions (EUDRAGIT® L 12.5, EUDRAGIT® E12.5, EUDRAGIT® RL 12.5/RS 12.5-12.5% organic solution).

When at least two melt-processed polymers are employed, one is preferably a cellulose derivative, more preferably a hydroxyalkylcellulose derivative, and optionally hydroxypropylmethylcellulose, and independently, the other polymer is preferably a (meth)acrylate polymer (such as, any suitable Eudragit polymer). Among the (meth)acrylate polymer polymers preferred in the context of the invention are Eudragit L and Eudragit RS. One more preferred polymer in the context of the invention is Eudragit RL. The Eudragit polymers can be used in combinations, with mixtures of Eudragit RS and RL being preferred.

Persons that (albeit inadvisedly) drink substantial quantities of alcoholic beverages when taking physician prescribed medications can substantially alter the composition of the gastric juices contained in the stomach, and in extreme cases these gastric juices can comprise up to 40% alcohol. Advantageously, embodiments of the inventive abuse-deterrent formulation optionally comprises a melt-processed mixture of at least one abuse-relevant drug, at least one cellulose ether or cellulose ester, and at least one (meth)acrylic polymer, wherein the amount of the drug that is extracted from the formulation by 20% aqueous ethanol, or 40% aqueous ethanol, or both, within one hour at 37° C. is less than or equal 1.5 times the amount of the drug that is extracted by 0.01 N hydrochloric acid within one hour at 37° C., or at 25° C. or both. The resistance to extraction by 40% ethanol is advantageous in those situations in which an individual purposefully attempts to extract an abuse relevant drug from a medicine containing an abuse relevant drug.

The protocols for extraction by 20% or 40% aqueous ethanol or 0.01 N hydrochloric acid, respectively, are given in the experimental section that follows. In more preferred embodiments, the amount of the drug that is extracted from the formulation by 20% or 40% aqueous ethanol is less than or equal 1.5 times the amount of the drug that is extracted by 0.01 N hydrochloric acid within one hour. In a yet more preferred embodiments, the amount of the drug that is extracted from the formulation by 20% or 40% aqueous ethanol is less than or equal the amount of the drug that is extracted by 0.01 N hydrochloric acid within one hour. In a yet more preferred embodiments, the amount of the drug that is extracted from the formulation by 20% or 40% aqueous ethanol is less than or equal 0.9 times the amount of the drug that is extracted by 0.01 N hydrochloric acid within one hour.

The present invention also provides a sustained release formulation of at least one abuse relevant drug that hampers the extraction of the drug from the formulation when extraction is by solvent extraction with commonly available household extraction solvents such as isopropyl alcohol, distilled alcohols exemplified by vodka, white vinegar, water and aqueous ethanol (e.g., 20% ethanol). Whereas the formulation is largely resistant to solvent-extraction, it still provides adequate drug release in aqueous solutions such as gastric fluids. This formulation when crushed or ground also provides adequate drug release in aqueous solutions such as gastric fluids. Fortunately, in certain preferred embodiments of the invention, the amount of the abuse relevant drug released from the time of placing in 3 oz. of one, or two, or three, or more than three, of the household solvents listed above (i.e., 0 hours) to 1 hour is expected to be not more than 15% greater than the amount released over the same time as when swallowed by an ordinary human, or the more than 1 hour to about 4 hours is not more than 15% greater than the amount released over the same time as when swallowed by an ordinary human, or both.

Exemplary preferred compositions of the invention comprise cellulose ethers and cellulose esters, which can be used alone or in combination in the invention have a preferable molecular weight in the range of 50,000 to 1,250,000 daltons. Cellulose ethers are preferably selected from alkylcelluloses, hydroxalkylcelluloses, hydroxyalkyl alkylcelluloses or mixtures therefrom, such as ethylcellulose, methylcellulose, hydroxypropyl cellulose (NF), hydroxyethyl cellulose (NF), and hydroxpropyl methylcellulose (USP), or combinations thereof. Useful cellulose esters are, without limitation, cellulose acetate (NF), cellulose acetate butyrate, cellulose acetate propionate, hydroxypropylmethyl cellulose phthalate, hydroxypropylmethyl cellulose acetate phthalate, and mixtures thereof. Most preferably, non-ionic polymers, such as hydroxypropylmethyl cellulose may be used.

The amount of substituent groups on the anhydroglucose units of cellulose can be designated by the average number of substituent groups attached to the ring, a concept known to cellulose chemists as “degree of substitution” (D. S.). If all three available positions on each unit are substituted, the D. S. is designated as 3, if an average of two on each ring are reacted, the D. S. is designated as 2, etc.

In preferred embodiments, the cellulose ether has an alkyl degree of substitution of 1.3 to 2.0 and hydroxyalkyl molar substitution of up to 0.85.

In preferred embodiments, the alkyl substitution is methyl. Further, the preferred hydroxyalkyl substitution is hydroxpropyl. These types of polymers with different substitution degrees of methoxy- and hydroxypropoxy-substitutions are summarized listed in pharmacopoeias, e.g. USP under the name “Hypromellose”.

Methylcellulose is available under the brand name METHOCEL A. METHOCEL A has a methyl (or methoxyl) D. S. of 1.64 to 1.92. These types of polymers are listed in pharmacopoeias, e.g. USP under the name “Methylcellulose”.

A particularly preferred cellulose ether is hydroxpropyl methylcellulose. Hydroxpropyl methylcellulose is available under the brand name METHOCEL E (methyl D. S. about 1.9, hydroxypropyl molar substitution about 0.23), METHOCEL F (methyl D. S. about 1.8, hydroxypropyl molar substitution about 0.13), and METHOCEL K (methyl D. S. about 1.4, hydroxypropyl molar substitution about 0.21). METHOCEL F and METHOCEL K are preferred hydroxpropyl methylcelluloses for use in the present invention.

The acrylic polymer suitably includes homopolymers and copolymers (which term includes polymers having more than two different repeat units) comprising monomers of acrylic acid and/or alkacrylic acid and/or an alkyl(alk)acrylate. As used herein, the term “alkyl(alk)acrylate” refers to either the corresponding acrylate or alkacrylate ester, which are usually formed from the corresponding acrylic or alkacrylic acids, respectively. In other words, the term “alkyl(alk)acrylate” refers to either an alkyl alkacrylate or an alkyl acrylate.

Preferably, the alkyl(alk)acrylate is a (C₁-C₂₂)alkyl((C₁-C₁₀)alk)acrylate. Examples of C₁-C₂₂ alkyl groups of the alkyl(alk)acrylates include methyl, ethyl, n-propyl, n-butyl, iso-butyl, tert-butyl, iso-propyl, pentyl, hexyl, cyclohexyl, 2-ethyl hexyl, heptyl, octyl, nonyl, decyl, isodecyl, undecyl, dodecyl, tridecyl, tetradecyl, pentadecyl, hexadecyl, heptadecyl, octadecyl, nonadecyl, eicosyl, behenyl, and isomers thereof. The alkyl group may be straight or branched chain. Preferably, the (C₁-C₂₂)alkyl group represents a (C₁-C₆)alkyl group as defined above, more preferably a (C₁-C₄)alkyl group as defined above. Examples of C₁₋₁₀ alk groups of the alkyl(alk)acrylate include methyl, ethyl, n-propyl, iso-propyl, n-butyl, iso-butyl, tert-butyl, pentyl, hexyl, cyclohexyl, 2-ethyl hexyl, heptyl, octyl, nonyl, decyl and isomers thereof. The alk groups may be straight or branched chain. Preferably, the (C₁-C₁₀)alk group represents a (C₁-C₆)alk group as defined above, more preferably a (C₁-C₄)alk group as defined above.

Preferably, the alkyl(alk)acrylate is a (C₁-C₄)alkyl((C₁-C₄)alk)acrylate, most preferably a (C₁-C₄)alkyl(meth)acrylate. It will be appreciated that the term (C₁-C₄)alkyl(meth)acrylate refers to either (C₁-C₄)alkyl acrylate or (C₁-C₄)alkyl methacrylate. Examples of (C₁-C₄)alkyl(meth)acrylate include methyl methacrylate (MMA), ethyl methacrylate (EMA), n-propyl methacrylate (PMA), isopropyl methacrylate (IPMA), n-butyl methacrylate (BMA), isobutyl methacrylate (IBMA), tert-butyl methacrylate (TBMA): methyl acrylate (MA), ethyl acrylate (EA), n-propyl acrylate (PA), n-butyl acrylate (BA), isopropyl acrylate (IPA), isobutyl acrylate (IBA), and combinations thereof.

Preferably, the alkacrylic acid monomer is a (C₁-C₁₀)alkacrylic acid. Examples of (C₁-C₁₀)alkacrylic acids include methacrylic acid, ethacrylic acid, n-propacrylic acid, iso-propacrylic acid, n-butacrylic acid, iso-butacrylic acid, tert-butacrylic acid, pentacrylic acid, hexacrylic acid, heptacrylic acid and isomers thereof. Preferably the (C₁-C₁₀)alkacrylic acid is a (C₁-C₄)alkacrylic acid, most preferably methacrylic acid.

In certain embodiments, the alkyl groups may be substituted by aryl groups. As used herein “alkyl” group refers to a straight chain, branched or cyclic, saturated or unsaturated aliphatic hydrocarbons. The alkyl group has 1-16 carbons, and may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl. A “hydroxy” group refers to an OH group. An “alkoxy” group refers to an —O-alkyl group wherein alkyl is as defined above. A “thio” group refers to an —SH group. A “thioalkyl” group refers to an —SR group wherein R is alkyl as defined above. An “amino” group refers to an —NH₂ group. An “alkylamino” group refers to an —NHR group wherein R is alkyl is as defined above. A “dialkylamino” group refers to an —NRR′ group wherein R and R′ are all as defined above. An “amido” group refers to an —CONH₂. An “alkylamido” group refers to an —CONHR group wherein R is alkyl is as defined above. A “dialkylamido” group refers to an —CONRR′ group wherein R and R′ are alkyl as defined above. A “nitro” group refers to an NO₂ group. A “carboxyl” group refers to a COOH group.

In certain embodiments, the alkyl groups may be substituted by aryl groups. As used herein, “aryl” includes both carbocyclic and heterocyclic aromatic rings, both monocyclic and fused polycyclic, where the aromatic rings can be 5- or 6-membered rings. Representative monocyclic aryl groups include, but are not limited to, phenyl, furanyl, pyrrolyl, thienyl, pyridinyl, pyrimidinyl, oxazolyl, isoxazolyl, pyrazolyl, imidazolyl, thiazolyl, isothiazolyl and the like. Fused polycyclic aryl groups are those aromatic groups that include a 5- or 6-membered aromatic or heteroaromatic ring as one or more rings in a fused ring system. Representative fused polycyclic aryl groups include naphthalene, anthracene, indolizine, indole, isoindole, benzofuran, benzothiophene, indazole, benzimidazole, benzthiazole, purine, quinoline, isoquinoline, cinnoline, phthalazine, quinazoline, quinoxaline, 1,8-naphthyridine, pteridine, carbazole, acridine, phenazine, phenothiazine, phenoxazine, and azulene. Also as used herein, aryl group also includes an arylalkyl group. Further, as used herein “arylalkyl” refers to moieties, such as benzyl, wherein an aromatic is linked to an alkyl group.

Preferably, the acrylic polymer is an acrylic copolymer. Preferably, the acrylic copolymer comprises monomers derived from alkyl(alk)acrylate, and/or acrylic acid and/or alkacrylic acid as defined hereinbefore. Most preferably, the acrylic copolymer comprises monomers derived from alkyl(alk)acrylate, i.e. copolymerisable alkyl acrylate and alkyl alkacrylate monomers as defined hereinbefore. Especially preferred acrylic copolymers include a (C₁-C₄)alkyl acrylate monomer and a copolymerisable (C₁-C₄)alkyl(C₁-C₄)alkacrylate comonomer, particularly copolymers formed from methyl methacrylate and a copolymerisable comonomer of methyl acrylate and/or ethyl acrylate and/or n-butyl acrylate.

Preferably, the (meth)acrylic polymer is a ionic (meth)acrylic polymer, in particular a cationic (meth)acrylic polymer. Ionic (meth)acrylic polymer are manufactured by copolymerising (meth)acrylic monomers carrying ionic groups with neutral (meth)acrylic monomers. The ionic groups preferably are quaternary ammonium groups.

The (meth)acrylic polymers are generally water-insoluble, but are swellable and permeable in aqueous solutions and digestive fluids. The molar ratio of cationic groups to the neutral (meth)acrylic esters allows for are control of the water-permeability of the formulation. In preferred embodiments the (meth)acrylic polymer is a copolymer or mixture of copolymers wherein the molar ratio of cationic groups to the neutral (meth)acrylic esters is in the range of about 1:20 to 1:35 on average. The ratio can by adjusted by selecting an appropriate commercially available cationic (meth)acrylic polymer or by blending a cationic (meth)acrylic polymer with a suitable amount of a neutral (meth)acrylic polymer.

Suitable (meth)acrylic polymers are commercially available from Rohm Pharma under the Tradename Eudragit, preferably Eudragit RL and Eudragit RS. Eudragit RL and Eudragit RS are copolymers of acrylic and methacrylic esters with a low content of quaternary ammonium groups, the molar ratio of ammonium groups to the remaining neutral (meth)acrylic esters being 1:20 in Eudragit RL and 1:40 in Eudragit RS. The mean molecular weight is about 150,000. Besides the (meth)acrylic polymers, further pharmaceutically acceptable polymers may be incorporated in the inventive formulations in order to adjust the properties of the formulation and/or improve the ease of manufacture thereof. These polymers may be selected from the group comprising: homopolymers of N-vinyl lactams, especially polyvinylpyrrolidone (PVP), copolymers of a N-vinyl lactam and one or more comonomers copolymerizable therewith, the comonomers being selected from nitrogen-containing monomers and oxygen-containing monomers; especially a copolymer of N-vinyl pyrrolidone and a vinyl carboxylate, preferred examples being a copolymer of N-vinyl pyrrolidone and vinyl acetate or a copolymer of N-vinyl pyrrolidone and vinyl propionate; polyvinyl alcohol-polyethylene glycol-graft copolymers (available as, e.g., Kollicoat® IR from BASF AG, Ludwigshafen, Germany); high molecular polyalkylene oxides such as polyethylene oxide and polypropylene oxide and copolymers of ethylene oxide and propylene oxide; polyacrylamides; vinyl acetate polymers such as copolymers of vinyl acetate and crotonic acid, partially hydrolyzed polyvinyl acetate (also referred to as partially saponified “polyvinyl alcohol”); polyvinyl alcohol; poly(hydroxy acids) such as poly(lactic acid), poly(glycolic acid), poly(3-hydroxybutyrate) and poly(3-hydroxybutyrate-co-3-hydroxyvalerate); or mixtures of one or more thereof. PVP generates hydrocodone N-oxide during extrusion, therefore use of PVP-polymers and -copolymers is not always preferred. However, when a small amount (0.2-0.6% w/w of the total formulation) of antioxidant is used, then PVP may be used preferably.

“Abuse-relevant drug” is intended to mean any biologically effective ingredient the distribution of which is subject to regulatory restrictions. Drugs of abuse that can be usefully formulated in the context of the invention include without limitation pseudoephedrine, anti-depressants, strong stimulants, diet drugs, steroids, and non-steroidal anti-inflammatory agents. In the category of strong stimulants, methamphetamine is one drug that has recently received popular attention as a drug of abuse. There is also some concern at the present time about the abuse potential of atropine, hyoscyamine, phenobarbital, scopolamine, and the like. Another major class of abuse-relevant drugs are analgesics, especially the opioids.

By the term “opioid,” it is meant a substance, whether agonist, antagonist, or mixed agonist-antagonist, which reacts with one or more receptor sites bound by endogenous opioid peptides such as the enkephalins, endorphins and the dynorphins Opioids include, without limitation, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvreturn, pentazocine, phenadoxone, phenazocine, phenomorphan, phenoperidine, piminodine, propiram, propoxyphene, sufentanil, tilidine, and tramadol, and salts and mixtures thereof.

In some preferred embodiments, the inventive formulation includes at least one additional therapeutic drug. In even more preferred embodiments, the additional therapeutic dug can be, without limitation, selected from the group consisting of non-steroidal, non-opioidal analgesics, and is optionally further selected from the group consisting of acetaminophen, aspirin, fentaynl, ibuprofen, indomethacin, ketorolac, naproxen, phenacetin, piroxicam, sufentanyl, sunlindac, and interferon alpha. Particularly preferred are those combinations of drug currently sold as fixed dose combinations to the public under the authority of a suitable national or regional regulatory agency, such as (by way of example) the U.S. Food and Drug Administration. Such drugs include without limitation a (fixed dose) combination of hydrocodone and acetaminophen, or a (fixed dose) combination of hydrocodone and ibuprofen.

The abuse-relevant drug(s) are preferably dispersed evenly throughout a matrix that is preferably formed by a cellulose ether or cellulose ester, and one acrylic or methacrylic polymer as well as other optional ingredients of the formulation. This description is intended to also encompass systems having small particles, typically of less than 1 μm in diameter, of drug in the matrix phase. These systems preferably do not contain significant amounts of active opioid ingredients in their crystalline or microcrystalline state, as evidenced by thermal analysis (DSC) or X-ray diffraction analysis (WAXS). At least 98% (by weight) of the total amount of drug is preferably present in an amorphous state. If additional non-abuse relevant drug actives like e.g. acetaminophen are additionally present in a formulation according to the present invention, this additional drug active(s) may be in a crystalline state embedded in the formulation.

When the dispersion of the components is such that the system is chemically and physically uniform or substantially homogenous throughout or consists of one thermodynamic phase, such a dispersion is called a “solid solution”. Solid solutions of abuse-relevant actives are preferred. The formulation can also comprise one or more additives selected from sugar alcohols or derivatives thereof, maltodextrines; pharmaceutically acceptable surfactants, flow regulators, disintegrants, bulking agents and lubricants. Useful sugar alcohols are exemplified by mannitol, sorbitol, xylitol; useful sugar alcohol derivatives include without limitation isomalt, hydrogenated condensed palatinose and others that are both similar and dissimilar.

Pharmaceutically acceptable surfactants are preferably pharmaceutically acceptable non-ionic surfactant. Incorporation of surfactants is especially preferred for matrices containing poorly water-soluble active ingredients and/or to improve the wettability of the formulation. The surfactant can effectuate an instantaneous emulsification of the active ingredient released from the dosage form and prevent precipitation of the active ingredient in the aqueous fluids of the gastrointestinal tract.

Some additives include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether or polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate or PEG-300 dioleate; alkylene glycol fatty acid mono esters, e.g. propylene glycol mono- and dilaurate (Lauroglycol®); sucrose fatty acid esters, e.g. sucrose monostearate, sucrose distearate, sucrose monolaurate or sucrose dilaurate; sorbitan fatty acid mono- and diesters such as sorbitan mono laurate (Span® 20), sorbitan monooleate, sorbitan monopalmitate (Span® 40), or sorbitan stearate, polyoxyethylene castor oil derivates, e.g. polyoxyethyleneglycerol triricinoleate or polyoxyl 35 castor oil (Cremophor® EL; BASF Corp.) or polyoxyethyleneglycerol oxystearate such as polyethylenglycol 40 hydrogenated castor oil (Cremophor® RH 40) or polyethylenglycol 60 hydrogenated castor oil (Cremophor® RH 60); or block copolymers of ethylene oxide and propylene oxide, also known as polyoxyethylene polyoxypropylene block copolymers or polyoxyethylene polypropyleneglycol such as Pluronic® F68, Pluronic® F127, Poloxamer® 124, Poloxamer® 188, Poloxamer® 237, Poloxamer® 388, or Poloxamer® 407 (BASF Wyandotte Corp.); or mono fatty acid esters of polyoxyethylene (20) sorbitan, e.g. polyoxyethylene (20) sorbitan monooleate (Tween® 80), polyoxyethylene (20) sorbitan monostearate (Tween® 60), polyoxyethylene (20) sorbitan monopalmitate (Tween® 40), polyoxyethylene (20) sorbitan monolaurate (Tween® 20), and the like as well as mixtures of two, three, four, five, or more thereof.

Various other additives may be included in the melt, for example flow regulators such as colloidal silica; lubricants, fillers, disintegrants, plasticizers, stabilizers such as antioxidants, light stabilizers, radical scavengers or stabilizers against microbial attack. Further, since the acetaminophen-containing overcoat layer has a bitter taste derived from acetaminophen itself, sweeteners and/or flavors etc. may be used as additives to reduce this bitter taste. One preferred way to reduce the bitter taste is a thin additional non-acetaminophen-containing overcoat.

The formulations of the invention can be obtained through any suitable melt process such as by the use of a heated press, and are preferably prepared by melt extrusion. In order to obtain a homogeneous distribution and a sufficient degree of dispersion of the drug, the drug-containing melt can be kept in the heated barrel of a melt extruder during a sufficient residence time. Melting occurs at the transition into a liquid or rubbery state in which it is possible for one component to be homogeneously embedded in the other. Melting usually involves heating above the softening point of meltable excipients of the formulation, e.g. a cellulose ether/ester, sugar alcohol and/or (meth)acrylic polymer. The preparation of the melt can take place in a variety of ways.

Usually, the melt temperature is in the range of 70 to 250° C., preferably 80 to 180° C., most preferably 100 to 140° C.

When the melt process comprises melt extrusion, the melting and/or mixing can take place in an apparatus customarily used for this purpose. Particularly suitable are extruders or kneaders. Suitable extruders include single screw extruders, intermeshing screw extruders, and multiscrew extruders, preferably twin screw extruders, which can be co-rotating or counterrotating and are optionally equipped with kneading disks. It will be appreciated that the working temperatures will also be determined by the kind of extruder or the kind of configuration within the extruder that is used. Part of the energy needed to melt, mix and dissolve the components in the extruder can be provided by heating elements. However, the friction and shearing of the material in the extruder may also provide the mixture with a substantial amount of energy and aid in the formation of a homogeneous melt of the components.

In another embodiment, the invention provides an oral, sustained release dosage form characterized in that it has at least two of the following features (a) the abuse relevant drug that is extracted from the formulation by ethanolic solvent, e.g. 40% or 20% aqueous ethanol or both within one hour at 37° C., with or without agitation, is less than or equal 1.5 times the amount of the abuse relevant drug that is extracted by 0.01 N hydrochloric acid within one hour at 37° C., (b) the dosage form is resistant to tampering and does not break under a force of 150 newtons, preferably 300 newtons, more preferably 450 newtons, yet more preferably 500 newtons as measured by “Pharma Test PTB 501” hardness tester, and (c) the dosage form releases at least 15%, more preferably 18%, and optionally 24% of the drug, but not more than 45%, more preferably 38% and optionally 34% of the drug during the 30 minutes, first hour, or first two hours in in vitro dissolution testing and optionally also in vivo (i.e., in the digestive tract of an animal or human). While not desiring to be bound by any particular theory, it is believed that high initial release rate of acetaminophen from the formulation is accomplished by providing a high drug load in the formulation, especially in the non-core region. Drug loading for a single active ingredient, such as acetaminophen in some embodiments of the inventive formulation can be greater than about 60%, 70%, 75%, 80%, 85%, by weight. The drug loading of acetaminophen can be limited to 80%.

A preferred embodiment of this dosage form is a monolithic form or a solid solution. The term “monolithic” is derived from roots meaning “single” and “stone”. A monolithic form or a solid preferably has at least one dimension that is more than 5 mm. In monolithic embodiments of the invention, the abuse relevant drug is preferably contained in a single solid, or a single solid solution, element. The monolithic solid or solid solution can optionally be overcoated or combined with other materials. These other materials preferably do not contain a substantial amount of the abuse relevant drug and these materials preferably do not substantially affect the rate of dissolution or dispersion of the abuse relevant drug in vivo or in vitro. The in vitro and/or in vivo release rates of the abuse relevant drug or abuse relevant drugs after about the first hour are preferably substantially constant for at least about 6, 8, 10, 12, or 16 hours. Thus, embodiments of the invention provides a single phase drug formulation that can be adapted to provide a burst of the abuse relevant drug(s) to allow therapeutic levels of the drug to be quickly obtained in the blood of a patient or animal, and to be maintained to provide therapeutic quantities for at least about 8, 12, or 24 hours. Additionally, the drug formulation is preferably suitable for repeated administration to a human or animal once, twice or three times a day. Advantageously, preferred embodiments of the inventive dosage form release substantially the entire quantity of the abuse relevant drug incorporated into the dosage form. For example, the inventive dosage form can be adapted to deliver greater than 90%, and preferably 95%, of the drug in in vitro dissolution testing within about 16, and optionally 12 or 9 hours. The cumulative blood concentration, or AUC, cannot be directly known from the time at which 90% of the drug is released from the formulation, however, in general higher AUCs per mg of the abuse relevant drug can be achieved when the drug formulation releases substantially all, or all, of the abuse relevant drug in portions of the digestive tract capable of absorbing the drug into the patient's (or animals) blood system.

In yet another preferred embodiment the invention provides a process for the manufacture of an abuse-resistant drug dosage formulation comprising melt extruding a formulation comprising at least one therapeutic drug further comprising directly shaping the extrudate into a dosage form without (an intermediate) milling step. The melt-extrudate preferably comprises a cellulose derivative, and preferably also comprises a Eudragit polymer. Preferred Eudragit polymers include Eudragit L or Eudragit RS or both, and particularly preferred is Eudragit RL or a combination of Eudragit RL and Eudragit RS.

The melt can range from pasty to viscous. Before allowing the melt to solidify, the melt optionally can be shaped into virtually any desired shape. Conveniently, shaping of the extrudate optionally can be carried out by a calendar, preferably with two counter-rotating rollers with mutually matching depressions on their surface. A broad range of tablet forms can be obtained by using rollers with different forms of depressions. Alternatively, the extrudate can be cut into pieces, either before (“hot-cut”) or after solidification (“cold-cut”) or used in a die injection process. Melt processes involving heated presses optionally can also be calendered.

The formed melt can be optionally overcoated with materials that do not contain substantial amount of the drug with abuse potential. For example, the monolithic dosage form containing the drug of abuse can be overcoated with a color coat, a swallowing aid, or another layer of pharmaceutically acceptable materials. The materials layered over the monolithic form preferably do not materially alter the rate of release of the active ingredient from the dosage form.

In order to facilitate the intake of such a dosage form by a mammal, it is advantageous to give the dosage form an appropriate shape. Large tablets that can be swallowed comfortably are therefore preferably elongated rather than round in shape.

A film coat on the dosage form further contributes to the ease with which it can be swallowed. A film coat also improves taste and provides an elegant appearance. If desired, the film coat may be an enteric coat. The film coat usually includes a polymeric film-forming material such as hydroxypropyl methylcellulose, hydroxypropylcellulose, and acrylate or methacrylate copolymers. Besides a film-forming polymer, the film-coat may further comprise a plasticizer, e.g. polyethylene glycol, a surfactant, e.g. a Tween® type, and optionally a pigment, e.g., titanium dioxide, iron oxides and/or sweeteners or flavors. The film-coating may also comprise talc as an anti-adhesive. The film coat usually accounts for less than about 5% by weight of the dosage form.

### EXEMPLARY EMBODIMENTS OF THE INVENTION

Certain exemplary embodiments of the present invention provide monolithic dosage formulations having biphasic release profile for readily water-soluble drugs having a polymer-containing tablet produced by extrusion and calendering. The formulations preferably have combination of immediate release and controlled release formulations of hydrocodone and acetaminophen compositions. These monolithic dosage formulation, especially having narcotic drugs may have abuse deterrent profiles such that the drug dissolution of the dosage forms has reduced/minimal dose dumping in 40% aqueous ethanol solution. Yet more preferably, these formulations may provide reproducible manufacturing processes offering options for rapid transfer to production scale.

The desired biphasic drug dissolution of acetaminophen can be achieved while retaining a monolithic dosage form by embedding the active ingredient (acetaminophen) in two formulations with differing release rates which are then combined to produce a two-layer or multi-layer tablet. Processes suitable for this purpose include coextrusion methods for the production of multilayer tablets as described in EP 0857062 specifically for extrudate dosage forms. One disadvantage of this technique is that two extruders have to be operated simultaneously and their mass and volume flows have to be coordinated with great exactness. Especially when shaping the tablet in the calender, the two melts have to be combined with each other in a ratio that is maintained very exactly to ensure compliance with the assay and content uniformity requirements of the tablets as specified in the pharmacopoeias (e.g. USP, Ph. Eur.). This requires a high level of effort.

It is also possible to manufacture the rapid release acetaminophen portion in a separate tablet which is then incorporated in the still plastic melt of the slow-releasing drug portion during calendering. After cooling, a calendered extruded tablet is obtained which contains a separately embedded rapid release component. Dosage forms of this type are described in U.S. Pat. No. 6,001,391 specifically for extruded dosage forms. One disadvantage of this approach is that the rapid release acetaminophen tablet has to be introduced very precisely into the individual calender cavities to prevent it being completely enveloped by the melt. Only if this rapid release acetaminophen component is located directly at the surface of the tablet can drug dissolution from this separate tablet portion start rapidly enough on contact with aqueous media.

It is also possible to obtain a rapid release acetaminophen component in the tablet by applying a film coating containing acetaminophen. The manufacture of film-coated extruded dosage forms is described in various patent applications. These patent applications do not however, describe a drug-containing film coating designed specifically to achieve biphasic drug dissolution. The results of the clinical study with an extruded dosage form produced in accordance with the patent application Ser. Nos. 11/625,705 and PCT/US07/60864

revealed that about 20% of the acetaminophen contained in the tablet have to be converted to a rapid release formulation to achieve the desired biphasic drug dissolution (for example, >about 30% after 1 h, >about 80% after 8 h). With a total acetaminophen content of about 500 mg per tablet, meant that about 100 mg of acetaminophen had to be rapidly released. Applying about 100 mg of an active ingredient in a rapid release form onto a tablets is difficult and only possible if certain requirements are fulfilled:
The drug content of the film-coating formulation must be very high so that the layers do not become too thick.
The drug-containing solution or dispersion used for film coating must have a high concentration to avoid long process times which would otherwise make the process uneconomical.
The film coating layer should also offer sufficient mechanical stability even with a large layer thickness, must not be tacky etc. and must be flexible enough that no cracking occurs even with thick layers. Good adhesion on the surface of the extruded cores must also be guaranteed.
The drug dissolution from the film-coating layer should also be rapid when using thick layers (about a maximum of 1 h in a preferred embodiment).
The organoleptic properties of the film-coating layer must also be largely unchanged with large layer thicknesses and during storage for extended periods of time at elevated temperature, high or very low relative humidity or a combination of such (i.e. no cracking, adhesion, chipping of the coating etc.).

Surprisingly, it has now been found that the above requirements can be fulfilled if finely ground acetaminophen is used for the film coating layers, together with relatively small amounts of a suitable water soluble or water-swellable polymer. It was found that formulations of this type with high active ingredient contents could be achieved, and that the viscosity of the spray solutions was conspicuously low even with very high total solids contents of more than 30% by weight, and that even thick film-coating layers (200 micrometers and more) could be applied in a relatively short time, thereby making the process economical. Drug dissolution was also sufficiently rapid in layers containing above 100 mg acetaminophen.

It was therefore possible to control very precisely the amount of acetaminophen sprayed on and thus also the drug dissolution profile (i.e. release during the first hour) via the layer thickness of the film coating.

Another surprising discovery was that the film coating formulations according to the invention were capable of very effectively smoothing the rough surfaces of the extruded tablets, i.e. the film coating sealed the indentations on the surface of the tablets very effectively. This was surprising considering that almost all commercially available film coatings and the polymers used to produce them actually do not possess and are not intended to possess this very property. Known polymers and film-coating formulations are designed to reproduce in detail the embossed elements (logos, etc.) and break lines in detail. In other words, “filling in” of the recesses present particularly in conventionally manufactured tablets is not desired and is to be absolutely avoided (see WO 2006/002808; particular reference is made to this fact in all the samples, see Example 4, page 18: “The embossing was well reproduced, without smearing and bridging effects”). Suitable polymers for the manufacture of the film-coating formulations are water soluble and water-swellable pharmaceutically accepted polymers which have already been used to date for the preparation of film coatings. The basic requirement is that sprayable, preferably purely aqueous solutions or suspensions are produced which have a total solids content (=sum of all the dissolved or suspended constituents including active ingredient) of at least 20% by weight (preferably 25%, particularly preferably 30% or more). The total solids content of the solution or dispersion must also have an active ingredient content of at least 50% (preferably 60%, particularly preferably 70% or higher). Non-aqueous solutions or suspensions are also possible if non-toxic, pharmaceutically accepted solvents such as ethanol are used. Mixtures of these organic solvents with water are also possible. In general, however, purely aqueous solutions or suspensions are preferred.

Particularly preferred are polymers which form comparatively low viscosity solutions in aqueous solution even at high concentrations in order to maintain the viscosity of the spray solution within the range in which an acceptable spray behavior of the solution or the suspension is still assured even when using the high total solids contents mentioned above. Suitable polymers include: non-ionic cellulose polymers such as hydroxypropyl cellulose, hydroxypropyl methylcellulose, hydroxyethyl cellulose; cationic polymethacrylates such as Eudragit® E, Eudragit® NE30D, Eudragit® RL, Eudragit® RS; polyvinyl alcohol; polyethylene oxide (high molecular polyethylene glycols with a molecular weight (MW)>100,000); polyvinyl alcohol/polyethylene oxide graft copolymers (Kollicoat® IR). Preferably, suitable polymers are selected from hydroxypropyl methylcellulose, Eudragit® NE30D and polyvinyl alcohol, or combinations thereof. More preferably, suitable polymers are polyvinyl alcohol/polyethylene oxide graft copolymers (e.g. Kollicoat® IR, BASF).

The active ingredient (preferred: acetaminophen) must either be soluble in the aforementioned high concentrations in the aqueous or aqueous/organic or purely organic solvents. If (as with acetaminophen) the aqueous solubility is not sufficient, preferably drug suspensions or dispersions can also be used. In this case, however, the decisive factor is that the particle size distribution of the active ingredient should be sufficiently fine since otherwise undesired, i.e. too rapid sedimentation of the suspended active ingredient in the spray solution occurs and/or the spray nozzles of the film coater become blocked. Preferred particle sizes are: not more than 10% of the particles above 0.25 mm (particularly preferred: not more than 5%), not more than 20% (particularly preferred not more than 10%) of the particles above 0.1 mm, and not more than 35% (particularly preferred not more than 20%) of the particles above 0.063 mm. To achieve this finer particle size, the drugs may be comminuted in grinding processes (dry and wet grinding are suitable).

Surprisingly, it was found that the film coating layers according to the invention not only adhere extremely well to the tablets but also do not become brittle or tacky and show no cracking even during storage at elevated temperatures of up to 60° C. There was also no detachment of the coating layer from the tablet core.

Various exemplary embodiments are depicted below. These Examples are being provided for illustrative purposes and they should not be deemed to narrow the scope of the invention.

### Example 1

### Manufacture of the Tablets for Film Coating

A homogeneous powder mixture consisting of 61.8% by weight acetaminophen, 12.6% by weight Eudragit® RL, 12.6% by weight xylitol, 6% by weight hydroxypropyl methylcellulose (Methocel® K100), 6% by weight hydroxypropyl methylcellulose (Methocel® K100M) and 1.0% by weight Aerosil® 200 was metered at a rate of 20 kg/h into a co-rotating twin screw extruder (ZSK-40) and extruded at a temperature of about 140° C. to produce a homogeneous, white melt ribbon. While still in the plastic state, this melt ribbon was introduced into the roll slit of a counter-rotating forming roller calender, the rollers of which had recesses on their surface from which tablets could be formed directly from the melt ribbon. The resulting tablets had a mean weight of 720 mg after cooling and deburring. The surface of the tablets was rough and uneven in places.

### Example 2

Acetaminophen with a particle size of 13% greater than 0.25 mm and 68% greater than 0.063 mm was suspended in water by stirring. The active ingredient settled very rapidly after switching off the stirrer. This suspension was comminuted and homogenized by passing through a colloidal mill. After milling, a solid, powdered polymer (Kollicoat® IR, BASF) was added to this suspension (mass ratio acetaminophen/Kollicoat® IR=75:25) to produce a total solids concentration of 30% by weight. Even after adding the polymer the acetaminophen still showed a marked tendency to sedimentation. While continuously stirring this suspension was then sprayed onto the tablets described in example 1 (6 kg) in a film coater (Driam). Samples of tablets were taken after 30, 50, 70 and 90 mg acetaminophen had been applied over the film coat. In all cases the coating was observed to adhere very well to the tablets, although the surface of the pure white film-coated tablets was still slightly rough due to the still relatively large acetaminophen particles. The loss on drying of the tablets was 1% by weight before and after film coating for all forms.

Film coating process parameters:

6 kg tablet cores
Drum speed: 12 rpm
Inlet air: 1200 m³/h
Inlet air temperature: 65° C.
Spraying rate: 40-45 g/min
Spraying pressure: 4,5 bar

### Example 3

Acetaminophen with a particle size of 1% greater than 0.25 mm, 5% greater than 0.1 mm and 16% greater than 0.063 mm was suspended in water by stirring. The active ingredient showed a decreased tendency to settle after switching off the stirrer compared to the material which was used in example 2. Solid, powdered polymer (Kollicoat® IR, BASF) was then added to this suspension (mass ratio acetaminophen/Kollicoat IR®=75:25) to produce a total solids concentration of 30% by weight. After adding the polymer, the acetaminophen showed hardly any tendency to settle. This suspension was then sprayed onto tablets (6 kg) which had been produced as described in Example 1 but with a slightly modified tablet geometry, in a film coater (Driam) (process parameters as in Example 2). The tablets were sampled after 30, 50, 70, 90 and 120 mg of acetaminophen had been applied to the film coat. Very good adhesion of the coating on the tablets was observed in all cases. The surface of the pure white film-coated tablets was smooth and uniform.

### Example 4

### Drug Dissolution of the Tablets

The drug dissolution of the tablets according to Example 1 was determined in an apparatus as per US Pharmacopoeia (USP Dissolution Apparatus II (Paddle), USP XXV; 37° C., 0.01 M HCl, 50 rpm). The amount of active ingredient released from the tablets into the aqueous HCl medium was determined by HPLC at different intervals.

Tablets without film coat application

Drug dissolution measured after 30 minutes: 7%
Drug dissolution measured after 60 minutes: 11%
Drug dissolution measured after 120 minutes: 17%
Drug dissolution measured after 240 minutes: 27%

### Example 5

### Drug Dissolution of the Film-Coated Tablets

The drug dissolution of the tablets according to Example 2 was determined in an apparatus as per US Pharmacopoeia (USP Dissolution Apparatus II (Paddle), USP XXV; 37° C., 0.01 M HCl, 50 rpm). The amount of active ingredient released from the tablets into the aqueous HCl medium was determined by HPLC at different intervals.

Film-coated tablet with 90 mg acetaminophen in the film coat:

Drug dissolution measured after 30 minutes: 16%
Drug dissolution measured after 60 minutes: 20%
Drug dissolution measured after 120 minutes: 27%
Drug dissolution measured after 240 minutes: 36%

The drug dissolution rates increased by about 10% at each test interval due to the initially rapid release of the active ingredient present in the film coat.

### Example 6

### Drug Dissolution of the Film-Coated Tablets

The drug dissolution of the tablets according to Example 3 was determined in an apparatus as per US Pharmacopoeia apparatus (paddle method, USP XXV; 37° C., 0.01 M HCl, 50 rpm). The amount of active ingredient released from the tablets into the aqueous HCl medium was determined by HPLC at different intervals.

Tablet without film coat application:

Drug dissolution measured after 30 minutes: 7%
Drug dissolution measured after 60 minutes: 12%
Drug dissolution measured after 120 minutes: 19%
Drug dissolution measured after 240 minutes: 29%
Drug dissolution measured after 360 minutes: 37%
Drug dissolution measured after 480 minutes: 43%

Film-coated tablet with 120 mg acetaminophen in the film coat:

Drug dissolution measured after 30 minutes: 28%
Drug dissolution measured after 60 minutes: 35%
Drug dissolution measured after 120 minutes: 43%
Drug dissolution measured after 240 minutes: 53%
Drug dissolution measured after 360 minutes: 62%
Drug dissolution measured after 480 minutes: 69%

The drug dissolution rates increased by about 25% at each test interval due to the rapid initial release of the active ingredient present in the film coat.

### Example 7

The test was performed as for Example 3, but instead of Kollicoat® IR a solid trituration based on hydroxypropyl methylcellulose was used which contained a small portion of iron oxide color pigments. Because of the markedly higher viscosity of the aqueous suspension the total solid concentration could only be adjusted to 20% by weight, as a result of which the spraying times increased while the other process parameters remained unchanged. Very good adhesion of the coating on the tablets was observed. The surface of the reddish/brownish film-coated tablets was smooth and uniform.

### Example 8

The test was performed as for Example 3, but instead of Kollicoat® IR a solid trituration based on polyvinyl alcohol was used which contained a small portion of titanium dioxide pigments. Because of the slightly higher viscosity of the aqueous suspension the total solid concentration could only be adjusted to 25% by weight, as a result of which the spraying times increased while the other process parameters remained unchanged. Very good adhesion of the coating on the tablets was observed. The surface of the pure white film-coated tablets was smooth and uniform.

### Example 9

Film tablets manufactured in accordance with Examples 3, 7 and 8 were stored in closed glass bottles at temperatures of 40° C. and 60° C. After 1 month no cracks were visible on the tablets and no tackiness was observed. Drug dissolution measured by the method described for Example 4 revealed no changes compared to the values recorded at the beginning of storage.

### Example 10

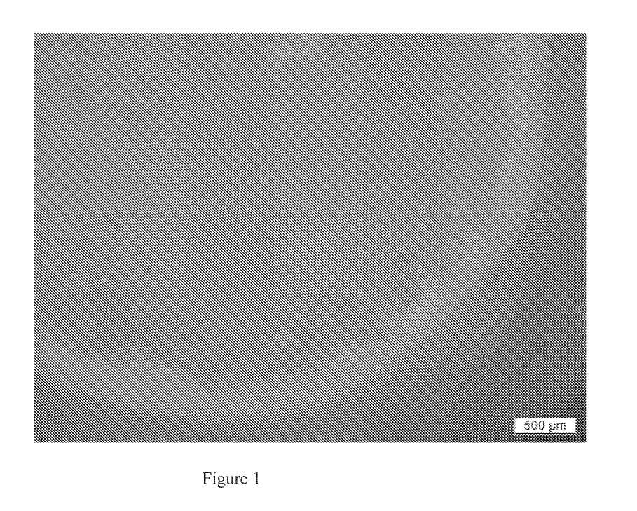

A film-coated tablet manufactured in accordance with Example 3 (90 mg acetaminophen in the film coating layer) was sampled and a thin section was taken in the transverse direction of the tablet with the aid of a microtome and examined under a microscope. The film coating layer was easily distinguishable from the tablet core in the images. The film coating layer was determined as being about 300 micrometers in the images. The smoothing effect of the coating suspension on the rough tablet surfaces was particularly evident, as also seen in FIGS. 1, 3 and 4.

### Example 11

### Dissolution in HCl and Aqueous Ethanol

Following is a description of exemplary methodology for studying rate of dissolution of certain compositions in HCl and 20% aqueous ethanol. Similar methodology may be used for studying rate of dissolution in 40% aqueous ethanol.

Following apparatus and procedures were use for dissolution in 0.01N hydrochloric acid and 20/40% aqueous ethanol:

### (I) Dissolution in 0.01 N HCl

### Apparatus: USP Dissolution Apparatus II (Paddle)

Rotation speed: 50 rpm

### Media: 0.01 N HCl

Media volume: 900 mL

### Temperature: 37° C.

Sampling time for 30 h release testing: 30/60/120/180/240/360/420/480/600/720/840/1080/1320/1560/1800 minutes
Sample volume: 10 mL (no volume replacement)
Sample preparation: used as is
Analytical finish: UV detection, wavelength 280 nm

### (II) Dissolution in 20 or 40% Aqueous Ethanol

### Apparatus: USP Dissolution Apparatus II (Paddle)

Rotation speed: 50 rpm
Media: 20 or 40% aqueous ethanol
Media volume 500 mL

### Temperature: 37° C.

Sampling time for 30 h release testing: 30/60/120/180/240/360/420/480/600/720/840/1080/1320/1560/1800 minutes
Sample volume: 10 mL (no volume replacement)
Sample preparation: used as is
Analytical finish: UV detection, wavelength 280 nm

### III. Dissolution Testing of Intact Tablets in 0.01 N HCl at 37° C.

a.) Fast releasing formulation (with respect to acetaminophen) in 0.01 N HCl at 37° C. is depicted in Table X. Table IX depicts the composition of the Core and the Overcoat of Formulation 5.

TABLE IX
Formulation 5:

| Core | Overcoat |
|---|---|
| 65.42% acetaminophen | 150 mg acetaminophen |
| 9.29% Eudragit RL-PO | 48 mg Kollicoat IR |
| 9.29% Hypromellose Ph. Eur. USP 2208 | |
| Type V 100 (Methocel K100) | |
| 9.29% Hydroxypropycellulose Ph. Eur. Type EF | |
| 2.99% Polaxamer 188 Ph. Eur./NF | |
| 2.8% hydrocodone | |
| 1% Aerosil 200 | |
| Total weight core: 535 mg | |
| Total weight coated tablet: 733 mg | |

Table X depicts dissolution data for hydrocodone (X(a)) and acetaminophen (X(b)).

TABLE X(a)

| Drug release hydrocodone | in 0.01N HCl |
|---|---|
| testing time point (min) | mean in % |
| 0 | 0 |
| 30 | 14 |
| 60 | 27 |
| 120 | 43 |
| 180 | 57 |
| 240 | 67 |
| 300 | 76 |
| 360 | 84 |
| 420 | 90 |
| 480 | 94 |
| 600 | 98 |
| 720 | 98 |
| 840 | 98 |
| 1080 | 99 |
| 1320 | 99 |
| 1560 | 99 |
| 1800 | 100 |

TABLE X(b)

| Drug release acetaminophen | in 0.01N HCl |
|---|---|
| testing time point (min) | mean in % |
| 0 | 0 |
| 30 | 33 |
| 60 | 39 |
| 120 | 46 |
| 180 | 56 |
| 240 | 64 |
| 300 | 71 |
| 360 | 78 |
| 420 | 85 |
| 480 | 90 |
| 600 | 98 |
| 720 | 100 |
| 840 | 101 |
| 1080 | 100 |
| 1320 | 100 |
| 1560 | 100 |
| 1800 | 100 |

b.) Slow releasing formulation (with respect to acetaminophen) in 0.01 N HCl at 37° C. is depicted in Table XII. Table XI depicts the composition of the Core and the Overcoat of Formulation 6.

TABLE XI
Formulation 6:

| Core | Overcoat |
|---|---|
| 55.88% acetaminophen | 120 mg acetaminophen |
| 13.50% Eudragit RL-PO | 38.4 mg Kollicoat IR |
| 11.0% Hypromellose Ph. Eur. USP 2208 | |
| Type V 100 (Methocel K100) | |
| 3.01% Hypromellose Ph. Eur. 2208 Type V | |
| 20000 (Methocel K100M) | |
| 13.40% Xylitol Ph. Eur./NF Typ Xylisorb 90 | |
| 2.21% hydrocodone | |
| 1% Aerosil 200 Ph. Eur./NF | |
| Total weight core: 680 mg | |
| Total weight coated tablet: 838.4 mg | |

Dissolution data for hydrocodone (XII(a)) and acetaminophen (XII(b)).

TABLE XII(a)

| Drug release hydrocodone | in 0.01N HCl |
|---|---|
| testing time point (min) | mean in % |
| 0 | 0 |
| 30 | 17 |
| 60 | 31 |
| 120 | 46 |
| 180 | 57 |
| 240 | 67 |
| 300 | 75 |
| 360 | 82 |
| 420 | 88 |
| 480 | 91 |
| 600 | 96 |
| 720 | 97 |
| 840 | 98 |
| 1080 | 99 |
| 1320 | 99 |
| 1560 | 99 |
| 1800 | 100 |

TABLE XII(b)

| Drug release acetaminophen | in 0.01N HCl |
|---|---|
| testing time point (min) | mean in % |
| 0 | 0 |
| 30 | 34 |
| 60 | 41 |
| 120 | 47 |
| 180 | 51 |
| 240 | 56 |
| 300 | 60 |
| 360 | 65 |
| 420 | 68 |
| 480 | 71 |
| 600 | 76 |
| 720 | 80 |
| 840 | 84 |
| 1080 | 89 |
| 1320 | 100 |
| 1560 | 100 |
| 1800 | 100 |

### IV. Dissolution Testing of Intact Tablets in 40% Aqueous Ethanol at 37° C.

a.) Fast releasing formulation (with respect to acetaminophen) in 40% aqueous ethanol at 37° C. is depicted in Table XIV. Table XIII depicts the composition of the Core and the Overcoat of Formulation 5.

TABLE XIII
Formulation 5:

| Core | Overcoat |
|---|---|
| 65.42% acetaminophen | 150 mg acetaminophen |
| 9.29% Eudragit RL-PO | 48 mg Kollicoat IR |
| 9.29% Hypromellose Ph. Eur. USP 2208 | |
| Type V 100 (Methocel K100) | |
| 9.29% Hydroxypropycellulose Ph. Eur. Type EF | |
| 2.99% Polaxamer 188 Ph. Eur./NF | |
| 2.8% hydrocodone | |
| 1% Aerosil 200 | |
| Total weight core: 535 mg | |
| Total weight coated tablet: 733 mg | |

Table XIV depicts dissolution data for hydrocodone (XIV(a)) and acetaminophen (XIV(b)).

TABLE XIV(a)

| Drug release hydrocodone | in 40% EtOH |
|---|---|
| testing time point (min) | mean in % |
| 0 | 0 |
| 30 | 15 |
| 60 | 33 |
| 120 | 56 |
| 180 | 77 |
| 240 | 90 |
| 300 | 97 |
| 360 | 97 |
| 420 | 97 |
| 480 | 98 |
| 600 | 98 |
| 720 | 99 |
| 840 | 100 |
| 1080 | 98 |
| 1320 | 99 |
| 1560 | 99 |
| 1800 | 100 |

TABLE XIV(b)

| Drug release acetaminophen | in 40% EtOH |
|---|---|
| testing time point (min) | mean in % |
| 0 | 0 |
| 30 | 31 |
| 60 | 51 |
| 120 | 67 |
| 180 | 82 |
| 240 | 93 |
| 300 | 98 |
| 360 | 99 |
| 420 | 101 |
| 480 | 101 |
| 600 | 101 |
| 720 | 101 |
| 840 | 101 |
| 1080 | 101 |
| 1320 | 101 |
| 1560 | 101 |
| 1800 | 102 |

b.) Slow releasing formulation (with respect to acetaminophen) in 40% aqueous ethanol at 37° C. is depicted in Table XVI. Table XV depicts the composition of the Core and the Overcoat of Formulation 8.

TABLE XV
formulation 8:

| Core | Overcoat |
|---|---|
| 55.88% acetaminophen | 120 mg acetaminophen |
| 13.50% Eudragit RL-PO | 38.4 mg Kollicoat IR |
| 11.0% Hypromellose Ph. Eur. USP 2208 Type V | |
| 100 (Methocel K100) | |
| 3.01% Hypromellose Ph. Eur. 2208 Type V | |
| 20000 (Methocel K100M) | |
| 13.40% Xylitol Ph. Eur./NF Typ Xylisorb 90 | |
| 2.21% hydrocodone | |
| 1% Aerosil 200 Ph. Eur./NF | |

Total weight core: 680 mg
Total weight coated tablet: 838.4 mg

Table XVI depicts dissolution data for hydrocodone (XVI(a)) and acetaminophen (XVI(b)).

TABLE XVI(a)

| Drug release hydrocodone | in 40% EtOH |
|---|---|
| testing time point (min) | mean in % |
| 0 | 0 |
| 30 | 12 |
| 60 | 24 |
| 120 | 38 |
| 180 | 51 |
| 240 | 62 |
| 300 | 72 |
| 360 | 80 |
| 420 | 85 |
| 480 | 91 |
| 600 | 96 |
| 720 | 99 |
| 840 | 100 |
| 1080 | 100 |
| 1320 | 102 |
| 1560 | 101 |
| 1800 | 100 |

TABLE XVI(b)

| Drug release acetaminophen | in 40% EtOH |
|---|---|
| testing time point (min) | mean in % |
| 0 | 0 |
| 30 | 23 |
| 60 | 38 |
| 120 | 47 |
| 180 | 57 |
| 240 | 65 |
| 300 | 73 |
| 360 | 80 |
| 420 | 84 |
| 480 | 90 |
| 600 | 94 |
| 720 | 98 |
| 840 | 100 |
| 1080 | 100 |
| 1320 | 101 |
| 1560 | 101 |
| 1800 | 102 |

### V. Dissolution Testing of Ground Tablets (Coffee Grinder 60 Sec) in 40% Aqueous Ethanol at 37° C.

In a household coffee grinder 3 extrudate tablet were milled for 60 sec at ˜20,000-50,000 rpm. The powder was collected and the to one tablet equivalent amount of powder was transferred to a dissolution vessel for release testing.

To determine the particle size analysis of the sample the powder was collected and sieved through a sieve with a mesh size of 355 μm. The material that went through the sieve was sieved again through a sieve with a mesh size of 63 μm. The following fractions were obtained:

Fraction 1: particle size >355 μm (˜20% of the total amount of powder)
Fraction 2: particle size >63 μm and <355 μm (˜66% of the total amount of powder)
Fraction 3: particle size <63 μm (˜14% of the total amount of powder)
a.) Fast releasing formulation (with respect to acetaminophen) in 40% aqueous ethanol at 37° C. is depicted in Table XVII. Dissolution data for hydrocodone (XVII(a)) and acetaminophen (XVII(b)) are depicted below:

TABLE XVII(a)

| Drug release hydrocodone | in 40% EtOH |
|---|---|
| testing time point (min) | mean in % |
| 0 | 0 |
| 30 | 56 |
| 60 | 75 |
| 120 | 92 |
| 180 | 99 |
| 240 | 101 |
| 300 | 101 |
| 360 | 100 |
| 420 | 101 |
| 480 | 100 |

TABLE XVII(b)

| Drug release acetaminophen | in 40% EtOH |
|---|---|
| testing time point (min) | mean in % |
| 0 | 0 |
| 30 | 51 |
| 60 | 69 |
| 120 | 87 |
| 180 | 94 |
| 240 | 97 |
| 300 | 97 |
| 360 | 97 |
| 420 | 97 |
| 480 | 97 |

b.) Slow releasing formulation (with respect to acetaminophen) in 40% aqueous ethanol at 37° C. is depicted in Table XVIII. Dissolution data for hydrocodone (XVIII(a)) and acetaminophen (XVIII(b)) are depicted below:

TABLE XVIII(a)

| Drug release hydrocodone | in 40% EtOH |
|---|---|
| testing time point (min) | mean in % |
| 0 | 0 |
| 30 | 42 |
| 60 | 56 |
| 120 | 74 |
| 180 | 84 |
| 240 | 91 |
| 300 | 96 |
| 360 | 98 |
| 420 | 100 |
| 480 | 100 |

TABLE XVIII(b)

| Drug release acetaminophen | in 40% EtOH |
|---|---|
| testing time point (min) | mean in % |
| 0 | 0 |
| 30 | 33 |
| 60 | 45 |
| 120 | 62 |
| 180 | 73 |
| 240 | 80 |
| 300 | 84 |
| 360 | 87 |
| 420 | 88 |
| 480 | 89 |

### VI. Dissolution Testing of Intact Tablets in 0.01 N HCl at 4° C.

a.) Fast releasing formulation (with respect to acetaminophen) in 0.01 N HCl at 4° C. is depicted in Table XIX. Dissolution data for hydrocodone (XIX(a)) and acetaminophen (XIX(b)) are depicted below:

TABLE XIX(a)

| Drug release hydrocodone | in 0.01N HCl |
|---|---|
| testing time point (min) | mean in % |
| 0 | 0 |
| 30 | 0 |
| 60 | 5 |
| 120 | 15 |
| 180 | 24 |
| 240 | 30 |
| 300 | 36 |
| 360 | 42 |
| 420 | 45 |
| 480 | 49 |

TABLE XIX(b)

| Drug release acetaminophen | in 0.01N HCl |
|---|---|
| testing time point (min) | mean in % |
| 0 | 0 |
| 30 | 16 |
| 60 | 23 |
| 120 | 30 |
| 180 | 34 |
| 240 | 36 |
| 300 | 39 |
| 360 | 41 |
| 420 | 43 |
| 480 | 44 |

b.) Slow releasing formulation (with respect to acetaminophen) in 0.01 N HCl at 4° C. is depicted in Table XX. Dissolution data for hydrocodone (XX(a)) and acetaminophen (XX(b)) are depicted below:

TABLE XX(a)

| Drug release hydrocodone | in 0.01N HCl |
|---|---|
| testing time point (min) | mean in % |
| 0 | 0 |
| 30 | 2 |
| 60 | 8 |
| 120 | 17 |
| 180 | 23 |
| 240 | 28 |
| 300 | 32 |
| 360 | 37 |
| 420 | 41 |
| 480 | 44 |

TABLE XX(b)

| Drug release acetaminophen | in 0.01N HCl |
|---|---|
| testing time point (min) | mean in % |
| 0 | 0 |
| 30 | 13 |
| 60 | 17 |
| 120 | 21 |
| 180 | 24 |
| 240 | 26 |
| 300 | 28 |
| 360 | 30 |
| 420 | 31 |
| 480 | 33 |

### VIII. Surface Roughness

Coating of the extrudated tablets resulted in significant smoothing of the tablet surface as can be seen in FIG. 1:

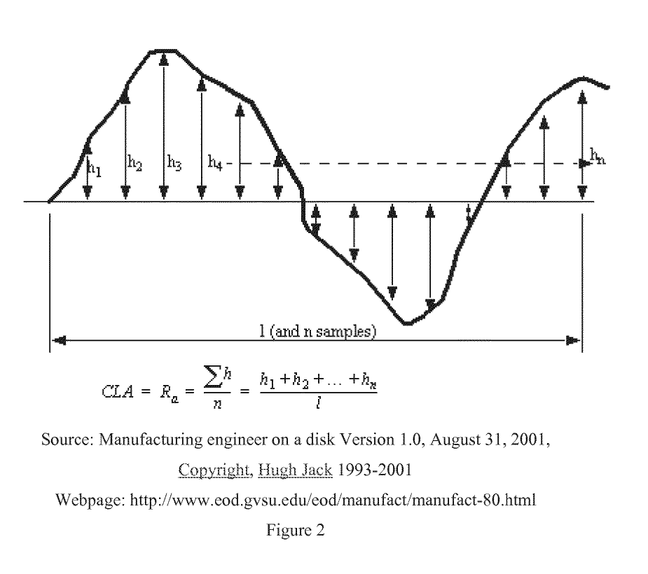

To determine the change in surface roughness coated and uncoated tablets were cut in half along the minor axis. The surface of this cross section was milled to obtain a plain and smooth surface. Optical micrographs of the cross section were used to determine the average surface roughness. For analysis, Centre Line Average approach (CLA), was used as depicted in FIG. 2, in which the average height per unit length off the centre line is determined. The centre line was put in the micrograph such that the area above and below the line are approximately equal.

The CLA is calculated by using samples at evenly spaced positions according to the following equation:

$$CLA = {R}_{a} = \frac{\sum h}{n} = \frac{{h}_{1} + {h}_{2} + … + {h}_{n}}{l}$$

The total length 1 was 4.69 mm, the distance between the increments was 68 μm.

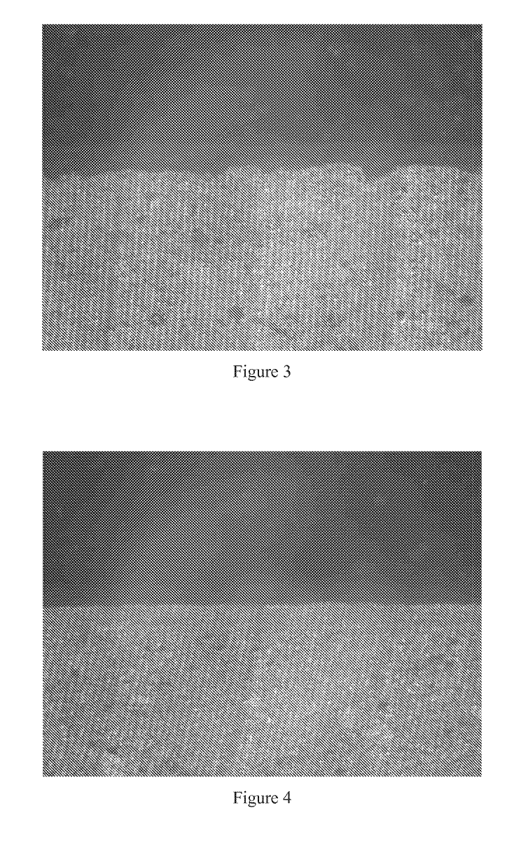

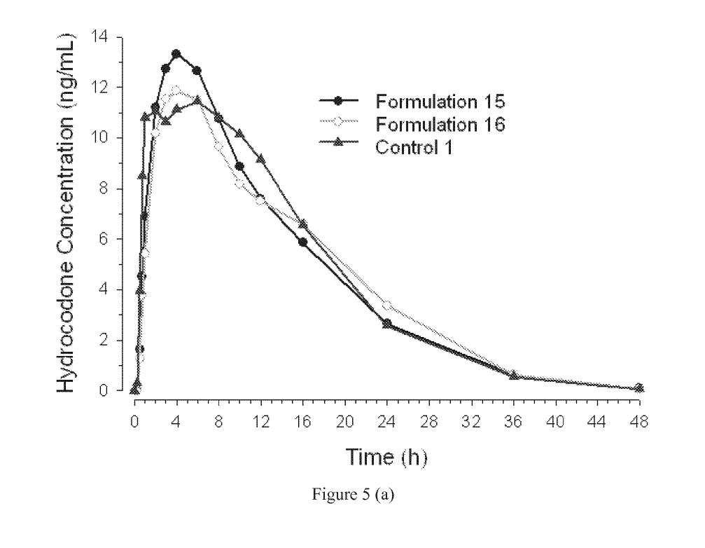

For uncoated formulation CLA=0.56, when (N=69), as shown in FIG. 3. Whereas for a coated formulation CLA=0.15, when (N=69), as shown in FIG. 4.

### IX. Dissolution Testing of Intact Tablets in 0.01 N HCl at 37° C. For Different Coating Thickness

a.) Slow releasing formulation (with respect to acetaminophen) in 0.01 N HCl at 37° C. is depicted for various Formulations 9-12 in Tables XXII and XXIII. Compositions of the Formulations are depicted in Table XXI.

TABLE XXI

| | Formulation | | | |
|---|---|---|---|---|
| | Formulation 9 | Formulation 10 | Formulation 11 | Formulation 12 |
| Composition | 60% | 60% | 60% | 60% acetaminophen |
| | acetaminophen | acetaminophen | acetaminophen | |
| | 12.6% | 12.6% Eudragit | 12.6% Eudragit | 12.6% Eudragit RL-PO |
| | Eudragit RL- | RL-PO | RL-PO | |
| | PO | | | |
| | 6.0% | 6.0% | 6.0% | 6.0% Hypromellose Ph. Eur. |
| | Hypromellose | Hypromellose Ph. | Hypromellose Ph. | USP 2208 Type V |
| | Ph. Eur. USP | Eur. USP 2208 | Eur. USP 2208 | 100(Methocel K100) |
| | 2208 Type V | Type V | Type V | |
| | 100(Methocel | 100(Methocel | 100(Methocel | |
| | K100) | K100) | K100) | |
| | 6.0% | 6.0% | 6.0% | 6.0% Hypromellose Ph. Eur. |
| | Hypromellose | Hypromellose Ph. | Hypromellose Ph. | 2208 Type V |
| | Ph. Eur. 2208 | Eur. 2208 Type V | Eur. 2208 Type V | 20000(Methocel K100M) |
| | Type V | 20000(Methocel | 20000(Methocel | |
| | 20000(Methocel | K100M) | K100M) | |
| | K100M) | | | |
| | 12.6% Xylitol | 12.6% Xylitol Ph. | 12.6% Xylitol Ph. | 12.6% Xylitol Ph. Eur./NF |
| | Ph. Eur./NF | Eur./NF Typ | Eur./NF Typ | Typ Xylisorb 90 |
| | Typ Xylisorb | Xylisorb 90 | Xylisorb 90 | |
| | 90 | | | |
| | 1.8% | 1.8% | 1.8% | 1.8% hydrocodone |
| | hydrocodone | hydrocodone | hydrocodone | |
| | 1% Aerosil | 1% Aerosil 200 | 1% Aerosil 200 | 1% Aerosil 200 Ph. Eur./NF |
| | 200 Ph. | Ph. Eur./NF | Ph. Eur./NF | |
| | Eur./NF | | | |
| Coating | | 50.0 mg | 85.0 mg | 120.0 mg acetaminophen |
| | | acetaminophen | acetaminophen | |
| | | 16.0 mg Kollicoat | 27.2 mg Kollicoat | 38.39 mg Kollicoat IR |
| | | IR | IR | |
| Target weight | 833 mg | 899 mg | 945.2 mg | 991.39 mg |

TABLE XXII

| Drug release | | | | |
|---|---|---|---|---|
| hydrocodone | Formulation | Formulation | Formulation | |
| testing point | 9 | 10 | 11 | Formulation 12 |
| (min) | mean in % | mean in % | mean in % | mean in % |
| 0 | 0 | 0 | 0 | 0 |
| 30 | 21 | 20 | 19 | 16 |
| 60 | 30 | 30 | 30 | 28 |
| 120 | 42 | 43 | 44 | 43 |
| 180 | 51 | 53 | 54 | 53 |
| 240 | 58 | 60 | 62 | 61 |
| 300 | 64 | 67 | 68 | 67 |
| 360 | 69 | 72 | 74 | 73 |
| 420 | 74 | 77 | 79 | 78 |
| 480 | 78 | 81 | 83 | 82 |

TABLE XXIII

| Drug release | | | | |
|---|---|---|---|---|
| acetaminophen | | | Formu- | Formu- |
| testing point | Formulation 9 | Formulation 10 | lation 11 | lation 12 |
| (min) | mean in % | mean in % | mean in % | mean in % |
| 0 | 0 | 0 | 0 | 0 |
| 30 | 7 | 15 | 19 | 22 |
| 60 | 11 | 19 | 23 | 26 |
| 120 | 17 | 25 | 29 | 32 |
| 180 | 22 | 29 | 33 | 36 |
| 240 | 26 | 33 | 37 | 40 |
| 300 | 30 | 36 | 40 | 43 |
| 360 | 33 | 39 | 42 | 45 |
| 420 | 36 | 42 | 45 | 48 |
| 480 | 39 | 45 | 48 | 51 |

X. Dissolution Testing of Intact Tablets without Overcoat in 0.01 N HCl at 37° C.
a.) Fast releasing formulation (with respect to acetaminophen) in 0.01 N HCl at 37° C. is depicted in Table XXV. Table XXIV depicts the composition of the Core of Formulation 13.

TABLE XXV
Formulation 13

| Core | No Overcoat |
|---|---|
| 65.42% acetaminophen | |
| 9.29% Eudragit RL-PO | |
| 9.29% Hypromellose Ph. Eur. USP 2208 Type V 100 | |
| (Methocel K100) | |
| 9.29% Hydroxypropycellulose Ph. Eur. Type EF | |
| 2.99% Polaxamer 188 Ph. Eur./NF | |
| 2.8% hydrocodone | |
| 1% Aerosil 200 | |
| Total weight: 535 mg | |

Dissolution data for hydrocodone (XXV(a)) and acetaminophen (XXV(b)) are depicted below:

TABLE XXV(a)

| Drug release hydrocodone | in 0.01N HCl |
|---|---|
| testing time point (min) | mean in % |
| 0 | 0 |
| 30 | 28 |
| 60 | 38 |
| 120 | 50 |
| 180 | 62 |
| 240 | 72 |
| 300 | 80 |
| 360 | 88 |
| 420 | 95 |
| 480 | 98 |
| 600 | 100 |
| 720 | 98 |
| 840 | 97 |
| 1080 | 97 |
| 1320 | 97 |
| 1560 | 97 |
| 1800 | 98 |

TABLE XXV(b)

| Drug release acetaminophen | in 0.01N HCl |
|---|---|
| testing time point (min) | mean in % |
| 0 | 0 |
| 30 | 13 |
| 60 | 19 |
| 120 | 27 |
| 180 | 41 |
| 240 | 54 |
| 300 | 66 |
| 360 | 79 |
| 420 | 88 |
| 480 | 95 |
| 600 | 105 |
| 720 | 106 |
| 840 | 104 |
| 1080 | 104 |
| 1320 | 104 |
| 1560 | 104 |
| 1800 | 104 |

b.) Slow releasing formulation (with respect to acetaminophen) in 0.01 N HCl at 37° C. is depicted in Table XXVII. Table XXVI depicts the composition of the Core of Formulation 13.

TABLE XXVI
Formulation 14

| Core | No Overcoat |
|---|---|
| 55.88% acetaminophen | |
| 13.50% Eudragit RL-PO | |
| 11.0% Hypromellose Ph. Eur. USP 2208 Type V 100 | |
| (Methocel K100) | |
| 3.01% Hypromellose Ph. Eur. 2208 Type V 20000 | |
| (Methocel K100M) | |
| 13.40% Xylitol Ph. Eur./NF Typ Xylisorb 90 | |
| 2.21% hydrocodone | |
| 1% Aerosil 200 Ph. Eur./NF | |
| Total weight: 680 mg | |

Dissolution data for hydrocodone (XXVII(a)) and acetaminophen (XXVII(b)) are depicted below:

TABLE XXVII(a)

| Drug release hydrocodone | in 0.01N HCl |
|---|---|
| testing time point (min) | mean in % |
| 0 | 0 |
| 30 | 30 |
| 60 | 42 |
| 120 | 54 |
| 180 | 65 |
| 240 | 72 |
| 300 | 79 |
| 360 | 88 |
| 420 | 94 |
| 480 | 96 |
| 600 | 99 |
| 720 | 101 |
| 840 | 100 |
| 1080 | 100 |
| 1320 | 100 |
| 1560 | 100 |
| 1800 | 100 |

TABLE XXVII(a)

| Drug release acetaminophen | in 0.01N HCl |
|---|---|
| testing time point (min) | mean in % |
| 0 | 0 |
| 30 | 11 |
| 60 | 17 |
| 120 | 25 |
| 180 | 31 |
| 240 | 36 |
| 300 | 42 |
| 360 | 48 |
| 420 | 53 |
| 480 | 56 |
| 600 | 63 |
| 720 | 69 |
| 840 | 74 |
| 1080 | 91 |
| 1320 | 99 |
| 1560 | 104 |
| 1800 | 103 |

### Example 12

### Compare Bioavailability of Test Formulations Against Control

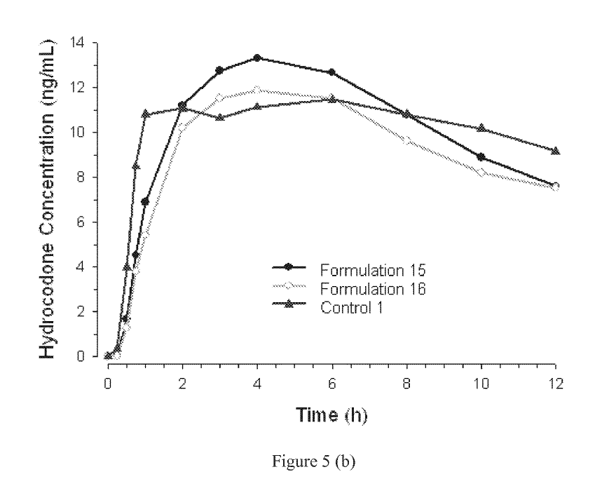

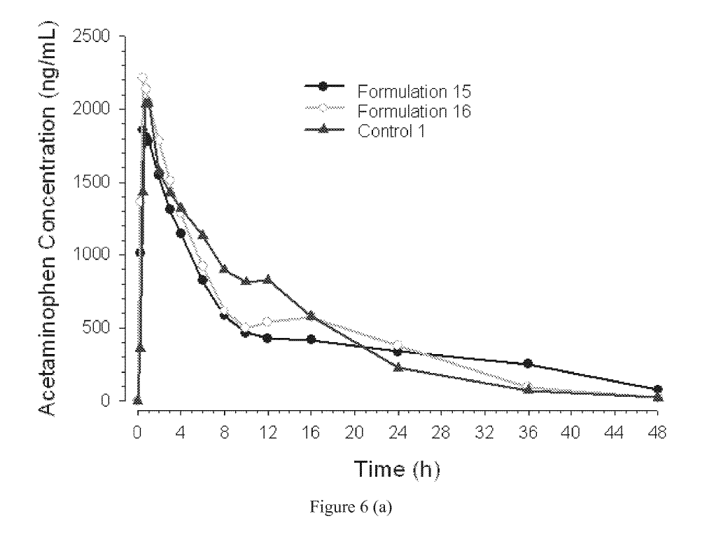

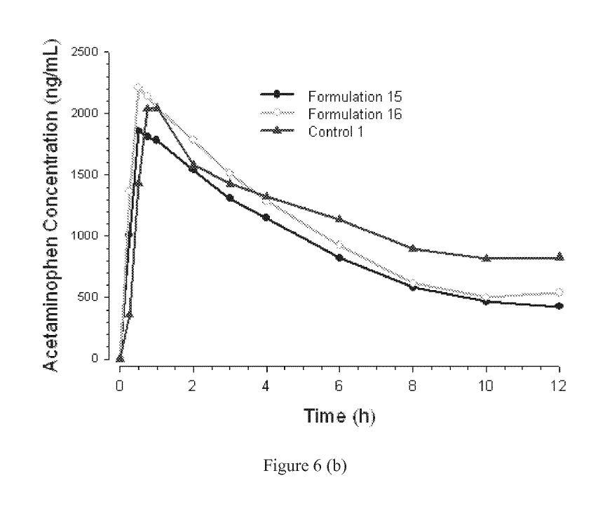

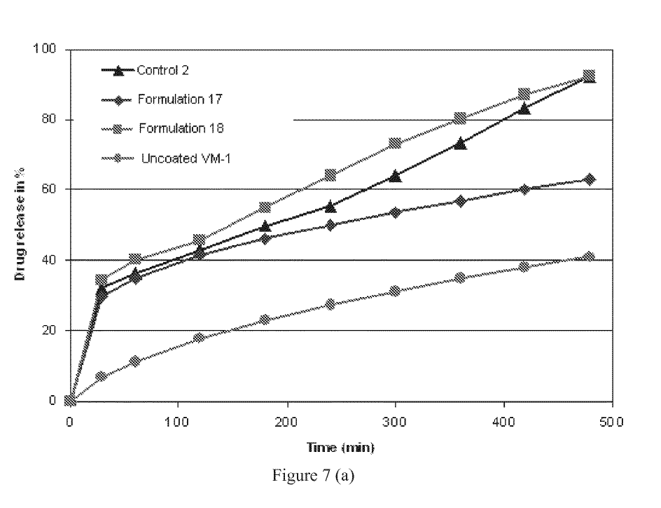

The objective of the study was to compare the bioavailability of two test formulations 15 and 16 with that of the reference Control table. The study design included single-dose, fasting, open-label, three-period, crossover study in 21 subjects. Regimen A included one tablet of Formulation 15; Regimen B included one tablet of Formulation 16; Regimen C included one tablet of Control 1. Blood samples were collected at 0, 0.25, 0.5, 0.75, 1, 2, 3, 4, 6, 8, 10, 12, 16, 24, 36 and 48 hours after the dose on Study Day 1. The following Table XXVIII illustrates compositions of test Formulations 15, 16 and Control 1. See also FIGS. 5 and 6 for mean hydrocodone and acetaminophen concentrations for Formulations 15, 16 and Control 1. Formulations 5, 7 and 15 are substantially identical to each other, however they have been numbered differently based on the different numbering of the tests and experiments. Similarly, formulations and 6, 8 and 16 are substantially identical to each other, however they have been numbered differently based on the different numbering of the tests and experiments. Also similarly Controls 1 and 2 are substantially identical to each other, however they have been numbered differently based on the different numbering of the tests and experiments.

In one embodiment of the invention, a preferred dosage form is Formulation 15 since Formulation 15 provides better blending properties than Formulation 16, both for blending of hydrocodone bitartrate pentahemihydrate and HPMC and blending of all components. Further, Formulation 15 blend provides for better flow properties than Formulation 16 into the extruder. Also Formulation 15 provides better direct shaping property than Formulation 16 since Formulation 15 is less sticky than Formulation 16. Moreover, Formulation 15 is expected to have better abuse deterrence than Formulation 16.

TABLE XXVIII

| Component | Test Formulations | | |
|---|---|---|---|
| Amount (mg)/Tablet | Formulation 15 | Formulation 16 | Control 1 |
| Tablet Core | | | |
| Hydrocodone Bitartrate | 15 | 15 | 10 |
| Acetaminophen | 380 | 350 | 330 |
| Tablet Overcoat | | | |
| Hydrocodone Bitartrate | — | — | 5 |
| Acetaminophen | 120 | 150 | 170 |

Preliminary pharmacokinetic parameters for Formulations 15, 16 and Control 1 are depicted below in Table XXIX:

TABLE XXIX

| Regimen | Pharmacokinetic Parameters | | | | | |
|---|---|---|---|---|---|---|
| | Hydrocodone (N = 20) | | | | | |
| | Tₘₐₓ | Cₘₐₓ | AUCₜ | AUC_{inf} | t_{1/2} | CL/F |
| | (h) | (ng/mL) | (ng * h/mL) | (ng * h/mL) | (h) | (L/h) |
| Formulation 15 | 4.4 | 14.0 | 205 | 209 | 6.22 | 44.7 |
| | (33%) | (17%) | (19%) | (18%) | (18%) | (19%) |
| Formulation 16 | 4.4 | 13.0 | 204 | 209 | 5.93 | 45.0 |
| | (32%) | (19%) | (20%) | (20%) | (22%) | (18%) |
| Control 1 | 4.8 | 12.6 | 211 | 214 | 5.68 | 43.5 |
| | (63%) | (20%) | (18%) | (18%) | (19%) | (16%) |
| | Acetaminophen (N = 20) | | | | | |
| | Tₘₐₓ | Cₘₐₓ | AUCₜ | AUC_{inf} | t_{1/2} | CL/F |
| | (h) | (μg/mL) | (μg * h/mL) | (μg * h/mL) | (h) | (L/h) |
| Formulation 15 | 0.74 | 2.06 | 21.2 | 22.9 | 9.85 | 24.0 |
| | (66%) | (25%) | (29%) | (30%) | (46%) | (33%) |
| Formulation 16 | 0.82 | 2.41 | 22.1 | 22.3 | 5.59 | 23.7 |
| | (82%) | (32%) | (24%) | (25%) | (21%) | (24%) |
| Control 1 | 0.83 | 2.23 | 22.1 | 22.4 | 6.47 | 23.7 |
| | (22%) | (24%) | (26%) | (26%) | (24%) | (24%) |

* N = 18

Preliminary relative bioavailability of Formulations 15 and 16 versus Control 1 is shown below in Table XXX:

TABLE XXX

| | | | | Relative Bioavailability | |
|---|---|---|---|---|---|
| Regimens | PK | Central Value* | | Point | 90% Confidence |
| Test vs. Reference | Parameter | Test | Reference | Estimate⁺ | Interval |
| | | Hydrocodone | | | |
| Formulation 15 vs. Control 1 | Cₘₐₓ | 13.950 | 12.626 | 1.105 | 1.040-1.173 |
| Formulation 16 vs. Control 1 | Cₘₐₓ | 13.240 | 12.626 | 1.049 | 0.985-1.116 |
| Formulation 15 vs. Control 1 | AUCₜ | 199.636 | 206.338 | 0.968 | 0.919-1.019 |
| Formulation 16 vs. Control 1 | AUCₜ | 203.905 | 206.338 | 0.988 | 0.937-1.042 |
| Formulation 15 vs. Control 1 | AUC_{∞} | 204.492 | 210.187 | 0.973 | 0.926-1.022 |
| Formulation 16 vs. Control 1 | AUC_{∞} | 208.867 | 210.187 | 0.994 | 0.944-1.046 |
| | | Acetaminophen | | | |
| Formulation 15 vs. Control 1 | Cₘₐₓ | 2.014 | 2.193 | 0.918 | 0.858-0.983 |
| Formulation 16 vs. Control 1 | Cₘₐₓ | 2.395 | 2.193 | 1.092 | 1.018-1.172 |
| Formulation 15 vs. Control 1 | AUCₜ | 20.580 | 21.732 | 0.947 | 0.899-0.998 |
| Formulation 16 vs. Control 1 | AUCₜ | 22.363 | 21.732 | 1.029 | 0.975-1.086 |
| Formulation 15 vs. Control 1 | AUC_{∞} | 22.171 | 21.987 | 1.008 | 0.944-1.077 |
| Formulation 16 vs. Control 1 | AUC_{∞} | 22.492 | 21.987 | 1.023 | 0.956-1.095 |

*Antilogarithm of the least squares means for logarithms.
+Antilogarithm of the difference (test minus reference) of the least squares means for logarithms.

Based on preliminary data, the two test Formulations 15 and 16 are bioequivalent to Control 1 with respect to both Cₘₐₓ and AUC_{∞}. The initial rate of hydrocodone absorption is slightly slower for test formulations 15 and 16 compared to Control 1.

### Example 13

### In Vitro Drug Release Profiles

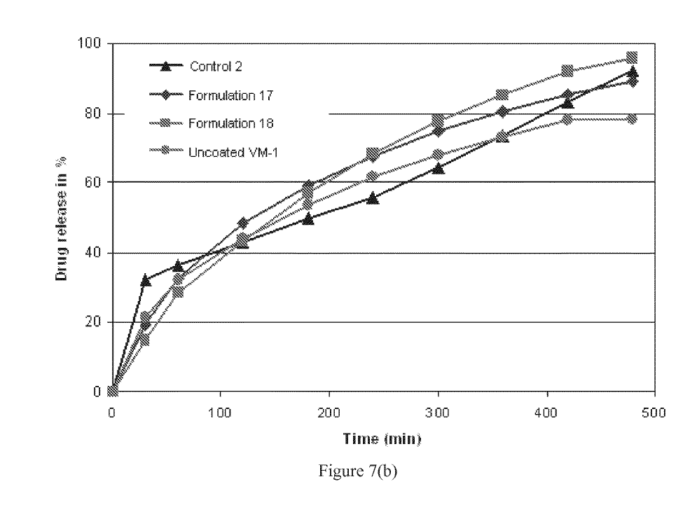

The following Formulations 17 and 18, as shown below in Table XXXI were studied for in vitro drug release profiles and this profile was compared with uncoated core VM-1 and Control 2, as shown in FIG. 7A and FIG. 7B.

TABLE XXXI

| | | | Formulation | Formulation |
|---|---|---|---|---|
| Component | | | 17 (650 mg) | 18 (500 mg) |
| Tablet | Quality Standard | Function | Amount (mg)/Tablet | |
| Hydrocodone Bitartrate | USP | Drug substance | 15.0 | 15.0 |
| | | | (2.2%) | (2.8%) |
| Acetaminophen | USP | Drug substance | 380.0 | 350.0 |
| | | | (55.9%) | (65.4%) |
| Eudragit ® RL-PO | NF/Ph. Eur. | Carrier polymer and | 91.8 | 49.7 |
| | | controlled release polymer | (13.5%) | (9.3%) |
| Hypromellose 2208, | USP/Ph. Eur. | Carrier polymer and | 74.8 | 49.7 |
| type V 100 | | controlled release polymer | (11.0%) | (9.3%) |
| Hypromellose 2208, | USP/Ph. Eur. | Carrier polymer and | 20.5 | — |
| type V 20000 | | controlled release polymer | (3.0%) | |
| Hydroxypropylcellulose, | Ph. Eur. | Carrier polymer and | — | 49.2 |
| type EF | | controlled release polymer | | (9.2%) |
| Xylitol | NF/Ph. Eur. | Release modifier | 91.1 | — |
| | | | (13.4%) | |
| Poloxamer 188 | NF/Ph. Eur. | Release modifier | — | 16.0 |
| | | | | (3.0%) |
| Colloidal silicon dioxide | NF/Ph. Eur. | Glidant | 6.8 | 5.4 |
| | | | (1.0%) | (1.0%) |
| Film Coating/Tablet wt. | | | 680 mg | 535 mg |
| Acetaminophen | USP | Drug substance | 120.0 | 150.0 |
| Kollicoat ® IR | In-house | Film former | 38.4 | 48.0 |
| Purified water | USP/Ph. Eur. | Solvent for film-coating | N/A | N/A |
| | | Coated Tablet Weight | 838.4 | 733.0 |

### Example 14

### Manufacturing of Tablets by Melt Extrusion, Deburring and Film-Coating

For each of the examples according to Table XXXII a homogeneous powder blend was prepared containing all ingredients. In the case of examples 14A to 16A a two-step blending was performed in order to ensure a homogeneous distribution of the low-dose API component (hydrocodon bitartrate 2.5 hydrate) in the final blend. Blending process is described in Table XXXIII. In the case of examples 14A-16A a total number of 5 powder samples from each final powder blend prior to extrusion were analyzed with respect to content uniformity of hydrocodone bitartrate 2.5. hydrate.

Table XXXII depicts composition of powder blends before extrusion and final extrudate tablet (after melt extrusion and direct shaping). All Ingredients were tested and released as specified according to US Pharmacopoeia (USP, NF) and/or European Pharmacopoeia (Ph. Eur.).

TABLE XXXII

| | | Example | Example | Example | Example |
|---|---|---|---|---|---|
| No. | Ingredient | 14A | 15A | 16A | 17A |
| 1 | Paracetamol Ph. Eur./USP | 55.9 | 65.4 | 60.0 | 61.8 |
| | (Acetaminophen) | | | | |
| 2 | Hydrocodon bitartrate 2.5 hydrate | 2.2 | 2.8 | 1.8 | — |
| 3 | Hypromellose Ph. Eur./USP 2208, | 11.0 | 9.3 | 6.0 | 6.0 |
| | Type V100 | | | | |
| | (Type: Methocel ® K100) | | | | |
| 4 | Hypromellose Ph. Eur./USP 2208, | 3.0 | — | 6.0 | 6.0 |
| | Type V20000 | | | | |
| | (Type: Methocel ® K100M) | | | | |
| 5 | Ammoniummethacrylat- | 13.5 | 9.3 | 12.8 | 12.6 |
| | Copolymer (Typ A) Ph. Eur./NF | | | | |
| | (Type: Eudragit RL PO) | | | | |
| 6 | Hydroxypropylcellulose Ph. Eur. | — | 9.2 | — | — |
| | (Type: Klucel ® EF) | | | | |
| 7 | Xylitol Ph. Eur./NF | 13.4 | | 12.6 | 12.6 |
| | (Type Xylisorb ® 90) | | | | |
| 8 | Poloxamer 188 Ph. Eur./NF | — | 3.0 | — | — |
| | (Type: Lutrol ® F68) | | | | |
| 9 | Colloidal silica P. Eur./NF | 1.0 | 1.0 | 1.0 | 1.0 |
| | (Type: Aerosil ® 200) | | | | |

TABLE XXXIII
Blending process for examples 14-17

| Step | Example 14B | Example 15B | Example 16B | Example 17B |
|---|---|---|---|---|
| 1 | Blending of #2, #3, | Blending of #2, #3, | Blending of #2, #3, | One-step-blending |
| | #4, #9 (according | #6, #9 (according | #4 (according to | of all ingredients |
| | to Table XXXII) | to Table XXXII) | Table XXXII) | according to Table |
| 2 | Adding #1, #5, #7 | Adding #1, #5, #8 | Adding #1, #5, #7, | XXXII |
| | (according to Table | (according to Table | #9 (according to | |
| | XXXII) to blend | XXXII) to blend | Table XXXII) to | |
| | from step 1. | from step 1. | blend from step 1. | |
| 3 | Blending the whole | Blending the whole | Blending the whole | |
| | mixture | mixture | mixture | |
| Total | 12 kg | 12 kg | 3 kg | 50 kg |
| batch | | | | |
| size | | | | |

The final blend from examples 14B-7B was dosed in a co-rotating twin-screw extruder at a constant feeding rate. The homogeneous, white drug-containing melt leaving the extruder nozzle was directly shaped into elongated tablets by calendering between two counterrotating rollers having depressions on their surface according to the dimensions listed in Table XXXIV. Process parameter settings of melt extrusion and calendering are listed in Table XXXIV.

Table XXXIV depicts melt extrusion and direct shaping (calendering) process:

TABLE XXXIV

| Process parameter | | | | |
|---|---|---|---|---|
| setting | Example 14C | Example 15C | Example 16C | Example 17C |
| Extruder | 18 mm | 18 mm | 18 mm | 40 mm |
| (screw diameter) | | | | |
| Tablet dimension | 19.0/6.9/3.0 mm | 20.0/5.9/2.5 mm | 17.5/7.97/7.6 mm | 19.0/6.9/3.0 mm |
| (calender roller | | | | |
| depression dimension) | | | | |
| (length/width/height) | | | | |
| Extrusion temperature | 129° C. | 124° C. | 140° C. | 140° C. |
| (melt temperature) | | | | |
| Calender temperature | 11° C. | 20° C. | 11° C. | 11° C. |
| Extrusion throughput | 1.5 kg/h | 1.5 kg/h | 1.5 kg/h | 25 kg/h |
| Batch size | 12 kg | 12 kg | 3 kg | 50 kg |

Tablets according to examples 14C, 15C and 17C were transferred into a Driam 600 film-coater. In a first step the tablets were tumbled in the coater at maximum rotation speed in order to polish the tablets and to remove the seems surrounding the tablets which derive from the calendering shaping process. This material which was removed from the tablets was removed from the coating drum together with the exhausting air. After this “deburring” step film-coating of the tablets was directly started in the same coater. In the case of example 16C tablets were placed in closed stainless steel container and tumbled for 10 minutes once removal of edges and seems was complete. Tablets were then dedusted on a sieve and transferred to the same Driam film-coater as in the case of the other examples. Composition of film-coating layer and process parameter settings of deburring step and of subsequent film-coating are listed in Table XXXV. Table XXXV depicts deburring of tablets after calendering

TABLE XXXV

| Process parameter | | | | |
|---|---|---|---|---|
| setting | Example 14D | Example 15D | Example 16D | Example 17D |
| Deburring time in | 20 min. | 94 min. | — | 60 min. |
| Driam film-coater | | | | |
| Deburring time in | — | — | 10 min. | — |
| stainless steel drum | | | | |
| Drum temperature | 25° C. | 25° C. | 25° C. | 25° C. |
| Tablet weight (mean) | 684.3 mg | 536.4 mg | 840.7 | 716 mg |
| after deburring | | | | |
| Acetaminophen drug | 382.5 mg | 350.8 mg | 500.4 mg | 442.5 mg |
| content per tablet | | | | |
| (calculated according to | | | | |
| composition and mean | | | | |
| tablet weight) | | | | |
| Hydrocodone | 15.0 mg | 15.0 mg | 15.1 mg | — |
| bitartrate 2.5 hydrate | | | | |
| drug content per tablet | | | | |
| (calculated according to | | | | |
| composition and mean | | | | |
| tablet weight) | | | | |
| Batch size | 4.9 kg | 6.5 kg | 1 kg | 7.8 kg |

Manufacturing of the film-coating suspension for examples 14E-16E was generally prepared by the following steps: First, acetaminophen was dispersed in water at room temperature during stirring. To this suspension the polymer (Kollicoat® IR) was added and stirring was continued until a homogeneous suspension was formed. This suspension was directly used for film-coating. Stirring was continued during the whole film-coating process. For examples 14E-17E a ready to use acetaminophen powder was used (Rhodia, acetaminophen “fine powder”). No additional sieving or micronizing was performed. Composition of film-coating suspensions are summarized in Table XXXVI.

Table XXXVI depicts composition of film-coating suspension

TABLE XXXVI

| | | | |
|---|---|---|---|
| Example | Example | Example | Example |
| 14E | 15E | 16E | 17E |

| | |
|---|---|
| Rel. amount of | 22.73% |
| acetaminophen | |
| Acetaminophen | 1% > 0.25 mm |
| particle size | 5% > 0.1 mm |
| (Rhodia [symbol] fine | 16% > 0.063 mm |
| powder [symbol] ) | |
| Rel. amount of | 7.27% |
| polymer (Type: | |
| Kollicoat ®IR) | |
| Rel. amount of water | 70.0% |
| (purified) | |

Film-coating of the deburred tablets was performed in a Driam 600 film-coater. Process conditions, parameter settings and data from final film-coated tablets are listed in Table XXXVII. In the case of all examples 14F-17F samples were taken at different time point during main phase of film-coating. This was to study the influence of different amount of coating layer thickness on drug release of both acetaminophen and hydrocodone bitartrate from the film-coated tablets. Spray rate during main phase of film-coating was at maximum rate of the peristaltic pump dosing the acetaminophen/Kollicoat® IR suspension. Higher spray rates should be possible.

Table XXXVII depicts film-coating process conditions

TABLE XXXVII

| Process | | | | |
|---|---|---|---|---|
| parameter | | | | |
| setting | Example 14F | Example 15F | Example 16F | Example 17F |
| | Pre-heating phase | | | |
| inlet air | 65° C. | | | |
| temperature | | | | |
| spray rate | — | | | |
| time | 10 min. | | | |
| | Starting phase 1 | | | |
| inlet air | 65° C. | | | |
| temperature | | | | |
| spray rate | 16 g/min. | | 15 g/min. | 10 g/min. |
| time | 5 min. | | 6 min. | 9 min. |
| | Starting phase 2 | | | |
| inlet air | 65° C. | | | |
| temperature | | | | |
| spray rate | 21 g/min. | | 20 g/min. | 25 g/min. |
| time | 10 min. | | 10 min. | 8 min. |
| | Main phase | | | |
| inlet air | 65° C. | | | |
| temperature | | | | |
| spray rate | 31-42 g/min. | 28-47 g/min. | 20-44 g/min. | 30-48 g/min. |
| time | 131 min. | 230 min. | 193 min. | 159 min. |
| | Drying/cooling phase | | | |
| inlet air | 25-30° C. | | | |
| temperature | | | | |
| spray rate | — | — | — | — |
| time | 5 min. | 5 min. | 5 min. | 5 min. |
| Batch size | 4.4 kg | 6.1 kg | 1 kg | 7 kg |
| Dimension of | 19.46 mm | 20.63 mm | 19.45 mm | 19.53 mm |
| film-coated | 7.82 mm | 7.32 mm | 10.66 mm | 7.62 mm |
| tablets (mean) | 7.07 mm | 6.41 mm | 7.71 mm | 7.23 mm |
| (length/width/ | | | | |
| height) | | | | |
| Weight of | 848.2 mg | 744.8 mg | 1018.4 mg | 872 mg |
| film-coated | | | | |
| tablets (mean) | | | | |
| Weight of | 157.9 mg | 208.4 mg | 177.7 mg | 156 mg |
| coating | | | | |
| layer per tablet | | | | |
| (calculated) | | | | |
| Acetaminophen | 119.6 mg | 157.9 mg | 134.6 mg | 118.2 mg |
| drug content | | | | |
| per film- | | | | |
| coated tablet | | | | |
| in film- | | | | |
| coating layer | | | | |
| (calculated) | | | | |
| Total | 502.1 mg | 508.7 mg | 635 mg | 560.7 mg |
| acetaminophen | | | | |
| drug content | | | | |
| per film- | | | | |
| coated tablet | | | | |
| (calculated) | | | | |
| Total | 15.0 mg | 15.0 mg | 15.1 mg | — |
| hydrocodone | | | | |
| bitartrate | | | | |
| 2.5 hydrate | | | | |
| drug content | | | | |
| per film- | | | | |
| coated tablet | | | | |
| (calculated) | | | | |

Generally, certain preferred embodiments of the present invention provide dosage forms and methods for the delivery of drugs, particularly drugs of abuse, characterized by resistance to solvent extraction; tampering, crushing or grinding, and providing an initial burst of release of drug followed by a prolonged period of controllable drug release.

Further, as shown below in Table XXXVIII, in one preferred embodiment, the present invention provides a pharmaceutical composition having a core and a non-core layer, comprising: (a) hydrocodone, a pharmaceutically acceptable salt or a hydrate thereof, and (b) acetaminophen or ibuprofen. In this embodiment, at least 75% all of the hydrocodone, pharmaceutically acceptable salt or hydrate thereof is in the core, and the acetaminophen or the ibuprofen is the non-core layer. Further, this composition is adapted so as to be useful for oral administration to a human 3, 2, or 1 times daily. Preferably, greater than 90% of the hydrocodone, pharmaceutically acceptable salt or hydrate thereof is in the core. More preferably, substantially all of the hydrocodone, pharmaceutically acceptable salt or hydrate thereof is in the core. In another embodiment, the core further comprises acetaminophen or ibuprofen. More preferably, the core further comprises acetaminophen.

TABLE XXXVIII

| | | | | | | | | | 95% CI | 95% CI | | |
|---|---|---|---|---|---|---|---|---|---|---|---|---|
| | | | | | | | | | Lower | Upper | Mean − | |
| PK parameter | Unit | Analyte | Regimen | N | Mean | SD | Min | Max | Mean | Mean | SD | Mean + SD |
| AUC0_1 | h * ug/mL | APAP | 848A | 21 | 1.38 | 0.38 | 0.90 | 2.20 | 1.21 | 1.56 | 1.01 | 1.76 |
| | | | 848B | 19 | 1.72 | 0.70 | 0.81 | 3.34 | 1.38 | 2.05 | 1.02 | 2.42 |
| | | | 851A | 16 | 0.32 | 0.13 | 0.14 | 0.65 | 0.25 | 0.39 | 0.19 | 0.45 |
| | h * ng/mL | HC | 848A | 21 | 2.37 | 1.37 | 0.82 | 6.68 | 1.75 | 3.00 | 1.01 | 3.74 |
| | | | 848B | 19 | 1.94 | 1.41 | 0.37 | 5.01 | 1.26 | 2.61 | 0.53 | 3.34 |
| | | | 851A | 16 | 2.71 | 1.19 | 1.35 | 5.93 | 2.08 | 3.35 | 1.52 | 3.90 |
| AUC0_2 | h * ug/mL | APAP | 848A | 21 | 3.06 | 0.68 | 2.20 | 4.61 | 2.75 | 3.37 | 2.38 | 3.74 |
| | | | 848B | 19 | 3.70 | 1.26 | 2.18 | 6.57 | 3.09 | 4.30 | 2.44 | 4.95 |
| | | | 851A | 16 | 1.00 | 0.32 | 0.53 | 1.73 | 0.83 | 1.17 | 0.68 | 1.32 |
| | h * ng/mL | HC | 848A | 21 | 11.4 | 3.8 | 6.9 | 21.8 | 9.7 | 13.2 | 7.62 | 15.25 |
| | | | 848B | 19 | 9.9 | 4.1 | 5.5 | 18.3 | 7.9 | 11.9 | 5.79 | 14.03 |
| | | | 851A | 16 | 11.3 | 2.9 | 7.6 | 18.2 | 9.8 | 12.9 | 8.43 | 14.20 |
| AUC0_3 | h * ug/mL | APAP | 848A | 21 | 4.51 | 1.00 | 2.97 | 6.76 | 4.06 | 4.96 | 3.51 | 5.51 |
| | | | 848B | 19 | 5.43 | 1.75 | 3.25 | 9.10 | 4.58 | 6.27 | 3.68 | 7.18 |
| | | | 851A | 16 | 1.75 | 0.53 | 1.02 | 2.89 | 1.47 | 2.04 | 1.22 | 2.28 |
| | h * ng/mL | HC | 848A | 21 | 23.5 | 6.0 | 15.6 | 38.2 | 20.8 | 26.2 | 17.5 | 29.5 |
| | | | 848B | 19 | 21.2 | 7.0 | 12.0 | 36.3 | 17.8 | 24.6 | 14.2 | 28.2 |
| | | | 851A | 16 | 22.1 | 4.7 | 16.2 | 32.2 | 19.6 | 24.6 | 17.4 | 26.8 |
| AUC0_4 | h * ug/mL | APAP | 848A | 21 | 5.77 | 1.31 | 3.59 | 8.60 | 5.17 | 6.37 | 4.46 | 7.08 |
| | | | 848B | 19 | 6.90 | 2.17 | 4.04 | 11.58 | 5.86 | 7.95 | 4.74 | 9.07 |
| | | | 851A | 16 | 2.52 | 0.73 | 1.48 | 3.97 | 2.14 | 2.91 | 1.80 | 3.25 |
| | h * ng/mL | HC | 848A | 21 | 36.7 | 8.2 | 25.9 | 54.8 | 32.9 | 40.4 | 28.5 | 44.8 |
| | | | 848B | 19 | 33.3 | 9.4 | 19.4 | 51.1 | 28.8 | 37.8 | 23.9 | 42.7 |
| | | | 851A | 16 | 33.7 | 6.6 | 24.1 | 45.5 | 30.2 | 37.3 | 27.1 | 40.3 |
| AUCinf | h * ug/mL | APAP | 848A | 21 | 23.2 | 6.9 | 11.0 | 35.9 | 20.1 | 26.3 | 16.3 | 30.1 |
| | | | 848B | 19 | 22.8 | 5.7 | 14.7 | 34.1 | 20.0 | 25.5 | 17.1 | 28.4 |
| | | | 851A | 16 | 25.3 | 12.0 | 12.0 | 49.3 | 18.9 | 31.7 | 13.2 | 37.3 |
| | h * ng/mL | HC | 848A | 21 | 208 | 38 | 129 | 306 | 191 | 225 | 170 | 245 |
| | | | 848B | 19 | 208 | 41 | 157 | 319 | 188 | 228 | 167 | 249 |
| | | | 851A | 16 | 229 | 48 | 135 | 322 | 203 | 255 | 181 | 277 |
| C1 | ug/mL | APAP | 848A | 21 | 1.80 | 0.42 | 1.17 | 2.75 | 1.60 | 1.99 | 1.38 | 2.22 |
| | | | 848B | 19 | 2.10 | 0.68 | 1.34 | 3.62 | 1.78 | 2.43 | 1.42 | 2.78 |
| | | | 851A | 16 | 0.61 | 0.19 | 0.29 | 0.93 | 0.51 | 0.72 | 0.42 | 0.81 |
| | | APAP/HC | 848A | 21 | 292 | 109 | 152 | 574 | 242 | 341 | 182 | 401 |
| | | | 848B | 19 | 462 | 247 | 221 | 1181 | 343 | 581 | 215 | 709 |
| | | | 851A | 16 | 90 | 24 | 58 | 134 | 77 | 103 | 66 | 115 |
| | ug/mL | APAP + HC | 848A | 21 | 1.80 | 0.42 | 1.18 | 2.76 | 1.61 | 2.00 | 1.38 | 2.23 |
| | | | 848B | 19 | 2.11 | 0.68 | 1.34 | 3.63 | 1.78 | 2.44 | 1.43 | 2.79 |
| | | | 851A | 16 | 0.62 | 0.19 | 0.29 | 0.94 | 0.52 | 0.72 | 0.43 | 0.81 |
| | ng/mL | HC | 848A | 21 | 6.86 | 2.80 | 2.95 | 13.70 | 5.58 | 8.13 | 4.06 | 9.65 |
| | | | 848B | 19 | 5.41 | 2.68 | 1.66 | 11.80 | 4.11 | 6.70 | 2.72 | 8.09 |
| | | | 851A | 16 | 6.96 | 1.90 | 3.93 | 10.10 | 5.95 | 7.97 | 5.06 | 8.86 |
| C12 | ug/mL | APAP | 848A | 21 | 0.44 | 0.14 | 0.22 | 0.71 | 0.37 | 0.50 | 0.30 | 0.58 |
| | | | 848B | 19 | 0.54 | 0.18 | 0.34 | 0.89 | 0.45 | 0.63 | 0.36 | 0.72 |
| | | | 851A | 16 | 0.45 | 0.12 | 0.25 | 0.68 | 0.39 | 0.52 | 0.33 | 0.57 |
| | | APAP/HC | 848A | 21 | 59.7 | 20.2 | 32.7 | 106 | 50.5 | 68.8 | 39.5 | 79.8 |
| | | | 848B | 19 | 74.0 | 22.0 | 45.2 | 138 | 63.4 | 84.6 | 52.0 | 96.0 |
| | | | 851A | 16 | 58.5 | 22.5 | 30.9 | 118 | 46.5 | 70.5 | 36.0 | 81.0 |
| | ug/mL | APAP + HC | 848A | 21 | 0.45 | 0.14 | 0.23 | 0.72 | 0.38 | 0.51 | 0.30 | 0.59 |
| | | | 848B | 19 | 0.55 | 0.18 | 0.34 | 0.91 | 0.46 | 0.63 | 0.36 | 0.73 |
| | | | 851A | 16 | 0.46 | 0.12 | 0.26 | 0.69 | 0.40 | 0.52 | 0.34 | 0.58 |
| | ng/mL | HC | 848A | 21 | 7.54 | 1.65 | 4.62 | 11.6 | 6.79 | 8.29 | 5.89 | 9.19 |
| | | | 848B | 19 | 7.38 | 1.80 | 4.87 | 13.3 | 6.52 | 8.25 | 5.58 | 9.19 |
| | | | 851A | 16 | 8.19 | 1.96 | 4.39 | 11.7 | 7.15 | 9.24 | 6.23 | 10.16 |
| C6 | ug/mL | APAP | 848A | 21 | 0.85 | 0.29 | 0.43 | 1.44 | 0.72 | 0.98 | 0.56 | 1.14 |
| | | | 848B | 19 | 0.97 | 0.34 | 0.40 | 1.82 | 0.80 | 1.14 | 0.63 | 1.31 |
| | | | 851A | 16 | 0.71 | 0.20 | 0.44 | 1.02 | 0.60 | 0.81 | 0.51 | 0.91 |
| | | APAP/HC | 848A | 21 | 66.1 | 16.7 | 38.4 | 98.6 | 58.5 | 73.7 | 49.4 | 82.8 |
| | | | 848B | 19 | 82.7 | 22.9 | 54.5 | 126 | 71.7 | 93.8 | 59.8 | 105.6 |
| | | | 851A | 16 | 57 | 17 | 35 | 91 | 48 | 66 | 39.7 | 73.4 |
| | ug/mL | APAP + HC | 848A | 21 | 0.86 | 0.29 | 0.45 | 1.45 | 0.73 | 1.00 | 0.57 | 1.16 |
| | | | 848B | 19 | 0.98 | 0.35 | 0.41 | 1.83 | 0.82 | 1.15 | 0.64 | 1.33 |
| | | | 851A | 16 | 0.72 | 0.20 | 0.45 | 1.04 | 0.61 | 0.83 | 0.52 | 0.92 |
| | ng/mL | HC | 848A | 21 | 12.8 | 2.2 | 8.2 | 16.0 | 11.8 | 13.8 | 10.6 | 14.9 |
| | | | 848B | 19 | 11.7 | 2.2 | 7.4 | 15.0 | 10.6 | 12.7 | 9.49 | 13.8 |
| | | | 851A | 16 | 12.8 | 3.0 | 8.7 | 19.3 | 11.2 | 14.4 | 9.83 | 15.8 |
| Cmax | ug/mL | APAP | 848A | 21 | 2.07 | 0.50 | 1.28 | 3.39 | 1.84 | 2.29 | 1.57 | 2.56 |
| | | | 848B | 19 | 2.46 | 0.79 | 1.58 | 4.40 | 2.08 | 2.84 | 1.67 | 3.24 |
| | | | 851A | 16 | 0.83 | 0.23 | 0.49 | 1.23 | 0.71 | 0.96 | 0.60 | 1.07 |
| | ng/mL | HC | 848A | 21 | 14.2 | 2.4 | 9.4 | 17.6 | 13.1 | 15.3 | 11.7 | 16.6 |
| | | | 848B | 19 | 13.4 | 3.1 | 8.7 | 21.1 | 11.9 | 14.9 | 10.4 | 16.5 |
| | | | 851A | 16 | 13.4 | 3.0 | 8.8 | 19.3 | 11.8 | 15.0 | 10.4 | 16.3 |
| Cmax/AUC | 1/h | APAP | 848A | 21 | 0.093 | 0.023 | 0.059 | 0.144 | 0.083 | 0.104 | 0.07 | 0.12 |
| | | | 848B | 19 | 0.107 | 0.015 | 0.081 | 0.129 | 0.1 | 0.115 | 0.09 | 0.12 |
| | | | 851A | 16 | 0.038 | 0.014 | 0.016 | 0.067 | 0.03 | 0.045 | 0.02 | 0.05 |
| | 1/h | HC | 848A | 21 | 0.069 | 0.012 | 0.052 | 0.098 | 0.064 | 0.075 | 0.06 | 0.08 |
| | | | 848B | 19 | 0.065 | 0.014 | 0.044 | 0.109 | 0.059 | 0.072 | 0.05 | 0.08 |
| | | | 851A | 16 | 0.059 | 0.009 | 0.048 | 0.076 | 0.054 | 0.064 | 0.05 | 0.07 |
| Cmax/C12 | | APAP | 848A | 21 | 5.0 | 1.4 | 2.7 | 8.9 | 4.4 | 5.7 | 3.60 | 6.42 |
| | | | 848B | 19 | 4.9 | 1.92 | 2.1 | 10.5 | 4.0 | 5.8 | 2.98 | 6.82 |
| | | | 851A | 16 | 1.9 | 0.60 | 1.2 | 3.2 | 1.6 | 2.2 | 1.30 | 2.50 |
| | | HC | 848A | 21 | 1.9 | 0.5 | 1.2 | 2.9 | 1.7 | 2.2 | 1.47 | 2.42 |
| | | | 848B | 19 | 1.9 | 0.7 | 1.0 | 4.1 | 1.6 | 2.2 | 1.24 | 2.56 |
| | | | 851A | 16 | 1.7 | 0.4 | 1.1 | 2.9 | 1.5 | 1.9 | 1.25 | 2.13 |
| Peak | h | APAP | 848A | 21 | 4.51 | 1.57 | 2.16 | 7.66 | 3.79 | 5.22 | 2.94 | 6.08 |
| width, 50* | | | 848B | 19 | 4.38 | 1.44 | 2.49 | 7.27 | 3.69 | 5.07 | 2.94 | 5.82 |
| | | | 851A | 16 | 20.5 | 11.2 | 7.2 | 44.4 | 14.6 | 26.5 | 9.34 | 31.74 |
| | h | HC | 848A | 21 | 12.4 | 3.2 | 7.5 | 18.0 | 10.9 | 13.8 | 9.15 | 15.57 |
| | | | 848B | 19 | 13.7 | 4.0 | 6.8 | 21.8 | 11.7 | 15.6 | 9.64 | 17.72 |
| | | | 851A | 16 | 14.6 | 3.4 | 9.5 | 19.8 | 12.8 | 16.4 | 11.2 | 18.0 |
| Tmax | h | APAP | 848A | 21 | 0.75 | 0.47 | 0.25 | 2.00 | 0.53 | 0.97 | 0.28 | 1.22 |
| | | | 848B | 19 | 0.93 | 0.82 | 0.25 | 3.00 | 0.54 | 1.33 | 0.11 | 1.75 |
| | | | 851A | 16 | 3.38 | 1.26 | 2.00 | 6.00 | 2.70 | 4.05 | 2.12 | 4.63 |
| | h | HC | 848A | 21 | 4.38 | 1.43 | 2.00 | 8.00 | 3.73 | 5.03 | 2.95 | 5.81 |
| | | | 848B | 19 | 4.37 | 1.42 | 2.00 | 6.00 | 3.68 | 5.05 | 2.95 | 5.79 |
| | | | 851A | 16 | 4.75 | 1.57 | 2.00 | 6.00 | 3.91 | 5.59 | 3.18 | 6.32 |
| AUC0_1/Dose | h * ng/mL/mg | APAP | 848A | 21 | 2.77 | 0.76 | 1.81 | 4.41 | 2.42 | 3.11 | 2.01 | 3.52 |
| | | | 848B | 19 | 3.43 | 1.40 | 1.62 | 6.69 | 2.76 | 4.11 | 2.03 | 4.83 |
| | | | 851A | 16 | 0.65 | 0.26 | 0.29 | 1.29 | 0.51 | 0.78 | 0.39 | 0.91 |
| | h * ng/mL/mg | HC | 848A | 21 | 0.261 | 0.151 | 0.090 | 0.735 | 0.193 | 0.330 | 0.111 | 0.412 |
| | | | 848B | 19 | 0.213 | 0.155 | 0.040 | 0.552 | 0.139 | 0.288 | 0.058 | 0.368 |
| | | | 851A | 16 | 0.298 | 0.131 | 0.149 | 0.653 | 0.229 | 0.369 | 0.167 | 0.430 |
| AUC0_2/Dose | h * ng/mL/mg | APAP | 848A | 21 | 6.12 | 1.36 | 4.40 | 9.22 | 5.50 | 6.74 | 4.76 | 7.48 |
| | | | 848B | 19 | 7.39 | 2.51 | 4.35 | 13.15 | 6.18 | 8.61 | 4.88 | 9.91 |
| | | | 851A | 16 | 2.00 | 0.65 | 1.05 | 3.45 | 1.66 | 2.35 | 1.36 | 2.65 |
| | h * ng/mL/mg | HC | 848A | 21 | 1.26 | 0.42 | 0.76 | 2.40 | 1.07 | 1.45 | 0.84 | 1.68 |
| | | | 848B | 19 | 1.09 | 0.45 | 0.60 | 2.02 | 0.87 | 1.31 | 0.64 | 1.55 |
| | | | 851A | 16 | 1.25 | 0.32 | 0.84 | 2.01 | 1.08 | 1.42 | 0.93 | 1.56 |
| AUC0_3/Dose | h * ng/mL/mg | APAP | 848A | 21 | 9.02 | 2.00 | 5.94 | 13.53 | 8.11 | 9.93 | 7.02 | 11.02 |
| | | | 848B | 19 | 10.85 | 3.50 | 6.50 | 18.21 | 9.17 | 12.54 | 7.36 | 14.35 |
| | | | 851A | 16 | 3.51 | 1.06 | 2.04 | 5.77 | 2.94 | 4.07 | 2.44 | 4.57 |
| | h * ng/mL/mg | HC | 848A | 21 | 2.59 | 0.66 | 1.72 | 4.21 | 2.29 | 2.89 | 1.93 | 3.25 |
| | | | 848B | 19 | 2.33 | 0.77 | 1.32 | 4.00 | 1.96 | 2.71 | 1.56 | 3.11 |
| | | | 851A | 16 | 2.44 | 0.52 | 1.79 | 3.54 | 2.16 | 2.71 | 1.92 | 2.96 |
| AUC0_4/Dose | h * ng/mL/mg | APAP | 848A | 21 | 11.54 | 2.62 | 7.19 | 17.21 | 10.35 | 12.74 | 8.92 | 14.17 |
| | | | 848B | 19 | 13.81 | 4.33 | 8.07 | 23.15 | 11.72 | 15.90 | 9.47 | 18.14 |
| | | | 851A | 16 | 5.04 | 1.45 | 2.95 | 7.94 | 4.27 | 5.82 | 3.59 | 6.49 |
| | h * ng/mL/mg | HC | 848A | 21 | 4.04 | 0.90 | 2.85 | 6.04 | 3.63 | 4.45 | 3.14 | 4.93 |
| | | | 848B | 19 | 3.66 | 1.03 | 2.14 | 5.63 | 3.17 | 4.16 | 2.63 | 4.70 |
| | | | 851A | 16 | 3.72 | 0.73 | 2.65 | 5.01 | 3.33 | 4.10 | 2.99 | 4.44 |
| AUCinf/Dose | h * ng/mL/mg | APAP | 848A | 21 | 46.4 | 13.7 | 22.1 | 71.8 | 40.1 | 52.6 | 32.6 | 60.1 |
| | | | 848B | 19 | 45.5 | 11.3 | 29.4 | 68.2 | 40.0 | 51.0 | 34.2 | 56.9 |
| | | | 851A | 16 | 50.6 | 24.1 | 24.0 | 98.7 | 37.7 | 63.4 | 26.5 | 74.6 |
| | h * ng/mL/mg | HC | 848A | 21 | 22.9 | 4.1 | 14.2 | 33.7 | 21.0 | 24.7 | 18.7 | 27.0 |
| | | | 848B | 19 | 22.9 | 4.5 | 17.3 | 35.1 | 20.7 | 25.1 | 18.4 | 27.4 |
| | | | 851A | 16 | 25.2 | 5.3 | 14.9 | 35.4 | 22.4 | 28.0 | 19.9 | 30.5 |
| C1/Dose | ng/mL/mg | APAP | 848A | 21 | 3.59 | 0.84 | 2.34 | 5.50 | 3.21 | 3.98 | 2.75 | 4.43 |
| | | | 848B | 19 | 4.21 | 1.36 | 2.68 | 7.24 | 3.55 | 4.86 | 2.85 | 5.57 |
| | | | 851A | 16 | 1.23 | 0.39 | 0.57 | 1.87 | 1.02 | 1.43 | 0.84 | 1.61 |
| | ng/mL/mg | HC | 848A | 21 | 0.75 | 0.31 | 0.32 | 1.51 | 0.61 | 0.90 | 0.45 | 1.06 |
| | | | 848B | 19 | 0.60 | 0.30 | 0.18 | 1.30 | 0.45 | 0.74 | 0.30 | 0.89 |
| | | | 851A | 16 | 0.77 | 0.21 | 0.43 | 1.11 | 0.66 | 0.88 | 0.56 | 0.98 |
| C12/Dose | ng/mL/mg | APAP | 848A | 21 | 0.88 | 0.29 | 0.44 | 1.43 | 0.75 | 1.01 | 0.59 | 1.17 |
| | | | 848B | 16 | 1.08 | 0.36 | 0.67 | 1.78 | 0.90 | 1.25 | 0.72 | 1.44 |
| | | | 851A | 19 | 0.90 | 0.24 | 0.50 | 1.35 | 0.78 | 1.03 | 0.66 | 1.14 |
| | ng/mL/mg | HC | 848A | 21 | 0.83 | 0.18 | 0.51 | 1.28 | 0.75 | 0.91 | 0.65 | 1.01 |
| | | | 848B | 16 | 0.81 | 0.20 | 0.54 | 1.46 | 0.72 | 0.91 | 0.61 | 1.01 |
| | | | 851A | 19 | 0.90 | 0.22 | 0.48 | 1.29 | 0.79 | 1.02 | 0.69 | 1.12 |
| C2/Dose | ng/mL/mg | APAP | 848A | 21 | 3.12 | 0.79 | 1.69 | 4.64 | 2.76 | 3.48 | 2.32 | 3.91 |
| | | | 848B | 19 | 3.71 | 1.21 | 2.08 | 6.40 | 3.13 | 4.30 | 2.50 | 4.92 |
| | | | 851A | 16 | 1.49 | 0.47 | 0.94 | 2.46 | 1.24 | 1.73 | 1.02 | 1.95 |
| | ng/mL/mg | HC | 848A | 21 | 1.24 | 0.30 | 0.78 | 1.82 | 1.10 | 1.38 | 0.94 | 1.54 |
| | | | 848B | 19 | 1.16 | 0.41 | 0.63 | 2.20 | 0.96 | 1.36 | 0.75 | 1.57 |
| | | | 851A | 16 | 1.13 | 0.22 | 0.82 | 1.60 | 1.01 | 1.25 | 0.91 | 1.35 |
| C3/Dose | ng/mL/mg | APAP | 848A | 21 | 2.68 | 0.74 | 1.38 | 3.98 | 2.34 | 3.01 | 1.94 | 3.41 |
| | | | 848B | 19 | 3.21 | 1.13 | 1.67 | 6.58 | 2.66 | 3.75 | 2.08 | 4.34 |
| | | | 851A | 16 | 1.52 | 0.43 | 0.83 | 2.18 | 1.29 | 1.75 | 1.09 | 1.95 |
| | ng/mL/mg | HC | 848A | 21 | 1.41 | 0.31 | 0.95 | 1.87 | 1.27 | 1.55 | 1.11 | 1.72 |
| | | | 848B | 19 | 1.33 | 0.35 | 0.81 | 2.32 | 1.16 | 1.49 | 0.98 | 1.67 |
| | | | 851A | 16 | 1.26 | 0.29 | 0.77 | 1.83 | 1.10 | 1.41 | 0.96 | 1.55 |
| C4/Dose | ng/mL/mg | APAP | 848A | 21 | 2.37 | 0.72 | 1.13 | 3.82 | 2.04 | 2.70 | 1.65 | 3.09 |
| | | | 848B | 19 | 2.69 | 0.84 | 1.47 | 4.92 | 2.29 | 3.10 | 1.85 | 3.54 |
| | | | 851A | 16 | 1.56 | 0.45 | 0.91 | 2.18 | 1.32 | 1.80 | 1.10 | 2.01 |
| | ng/mL/mg | HC | 848A | 21 | 1.49 | 0.29 | 1.03 | 1.94 | 1.35 | 1.62 | 1.20 | 1.77 |
| | | | 848B | 19 | 1.34 | 0.26 | 0.83 | 1.91 | 1.21 | 1.46 | 1.07 | 1.60 |
| | | | 851A | 16 | 1.30 | 0.24 | 0.96 | 1.81 | 1.17 | 1.43 | 1.06 | 1.54 |
| C6/Dose | ng/mL/mg | APAP | 848A | 21 | 1.70 | 0.58 | 0.87 | 2.88 | 1.44 | 1.97 | 1.12 | 2.29 |
| | | | 848B | 19 | 1.94 | 0.69 | 0.81 | 3.64 | 1.61 | 2.27 | 1.25 | 2.63 |
| | | | 851A | 16 | 1.41 | 0.40 | 0.87 | 2.04 | 1.20 | 1.63 | 1.02 | 1.81 |
| | ng/mL/mg | HC | 848A | 21 | 1.40 | 0.24 | 0.90 | 1.76 | 1.30 | 1.51 | 1.16 | 1.65 |
| | | | 848B | 19 | 1.28 | 0.24 | 0.81 | 1.65 | 1.17 | 1.40 | 1.04 | 1.52 |
| | | | 851A | 16 | 1.41 | 0.33 | 0.96 | 2.13 | 1.24 | 1.58 | 1.08 | 1.74 |
| Cmax/Dose | ng/mL/mg | APAP | 848A | 21 | 4.13 | 1.00 | 2.56 | 6.78 | 3.68 | 4.59 | 3.14 | 5.13 |
| | | | 848B | 19 | 4.91 | 1.57 | 3.16 | 8.80 | 4.16 | 5.67 | 3.34 | 6.49 |
| | | | 851A | 16 | 1.66 | 0.47 | 0.97 | 2.46 | 1.41 | 1.91 | 1.19 | 2.13 |
| | ng/mL/mg | HC | 848A | 21 | 1.56 | 0.27 | 1.03 | 1.94 | 1.44 | 1.68 | 1.29 | 1.83 |
| | | | 848B | 19 | 1.48 | 0.34 | 0.96 | 2.32 | 1.31 | 1.64 | 1.14 | 1.81 |
| | | | 851A | 16 | 1.47 | 0.33 | 0.97 | 2.13 | 1.30 | 1.65 | 1.15 | 1.80 |

*estimated as total time above 50% of Cmax value

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma profile characterized by a Cmax for hydrocodone from about 0.6 ng/mL/mg to about 1.4 ng/mL/mg and a Cmax for acetaminophen from about 2.8 ng/mL/mg and 7.9 ng/mL/mg after a single dose. In another embodiment, the pharmaceutical composition produces a plasma profile characterized by a Cmax for hydrocodone of about 0.4 ng/mL/mg to about 1.9 ng/mL/mg and a Cmax for acetaminophen of about 2.0 ng/mL/mg to about 10.4 ng/mL/mg after a single dose. In yet another embodiment, the pharmaceutical composition produces a plasma profile characterized by a Cmax for hydrocodone of from about 0.6 ng/mL/mg to about 1.0 ng/mL/mg and a Cmax for acetaminophen of from about 3.0 ng/mL/mg to about 5.2 ng/mL/mg after a single dose. Other embodiments of the dosage form include about 5-20 mg of hydrocodone bitartrate pentahemihydrate and about 400-600 mg of acetaminophen. Yet another embodiment of the dosage form includes 10-15 mg of hydrocodone bitartrate pentahemihydrate and about 500-600 mg of acetaminophen.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. When administered to the human patient, the dosage form produces an AUC for hydrocodone of about 9.1 ng*hr/mL/mg to about 19.9 ng*hr/mL/mg and an AUC for acetaminophen of about 28.6 ng*hr/mL/mg to about 59.1 ng*hr/mL/mg. In another embodiment, the dosage form produces an AUC for hydrocodone of about 7.0 ng*hr/mL/mg to about 26.2 ng*hr/mL/mg and an AUC for acetaminophen of about 18.4 ng*hr/mL/mg to about 79.9 ng*hr/mL/mg. In yet another embodiment, the dosage form produces an AUC for hydrocodone of about 11.3 ng*hr/mL/mg to about 18.7 ng*hr/mL/mg and an AUC for acetaminophen of about 28.7 ng*hr/mL/mg to about 53.5 ng*hr/mL/mg. Preferably in this embodiment, the in vitro rate of release of the pharmaceutical composition has a biphasic release profile, and wherein for each phase of the in vitro rate of release is zero order or first order for acetaminophen and zero order or first order for hydrocodone bitartrate pentahemihydrate.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone of about 0.18 ng/mL/mg to about 1.51 ng/mL/mg, and a plasma concentration at 1 hour Cl for acetaminophen of about 2.34 ng/mL/mg to about 7.24 ng/mL/mg. In preferred embodiments such as Formulation 15, the dosage form produces a Cl for hydrocodone of about 0.32 ng/mL/mg to about 1.51 ng/mL/mg and a Cl for acetaminophen of about 2.34 ng/mL/mg to about 5.50 ng/mL/mg.

In certain other embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone from about 0.30 ng/mL/mg to about 1.06 ng/mL/mg, and a Cl for acetaminophen from about 2.75 ng/mL/mg to about 5.57 ng/mL/mg. In preferred embodiments, the dosage from produces a Cl for hydrocodone from about 0.45 ng/mL/mg to about 1.06 ng/mL/mg and a Cl for acetaminophen from about 2.75 ng/mL/mg to about 4.43 ng/mL/mg.

In certain embodiments, the dosage form produces a combined Cl for hydrocodone and acetaminophen from about 1.18 μg/mL to about 3.63 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.18 μg/mL to about 2.76 μg/mL, after a single dose of 15 mg hydrocodone bitartrate pentahemihydrate and 500 mg of acetaminophen.

In certain embodiments, the dosage form produces a combined Cl for hydrocodone and acetaminophen from about 1.38 μg/mL to about 2.79 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.38 μg/mL to about 2.23 μg/mL, after a single dose of 15 mg hydrocodone bitartrate pentahemihydrate and 500 mg of acetaminophen.

In preferred embodiments, the dosage form produces a combined Cl for hydrocodone and acetaminophen of 1.80±0.42 μg/mL with the 95% confidence interval for the mean value falling between about 1.61 μg/mL to about 2.00 μg/mL, after a single dose of 15 mg hydrocodone bitartrate pentahemihydrate and 500 mg of acetaminophen. The 95% confidence interval of combined Cl for hydrocodone and acetaminophen for the preferred embodiments and the Control overlapped. The 95% confidence interval for the mean value of combined Cl for hydrocodone and acetaminophen for the Control ranged from about 1.46 to 1.96 μg/mL, after administered as a single dose of 15 mg hydrocodone bitartrate pentahemihydrate and 500 mg of acetaminophen to the human patient. The Control provides sufficient plasma levels of opioid and nonopioid analgesic to provide a reduction in pain intensity within about 1 hour after administration.

When administered to a population of healthy North Americans or Western Europeans, particularly when the formulation is adapted to be suitable for, or intended for, administration to a human every 12 hours as needed, about 20-45% of the hydrocodone is released in vitro from the pharmaceutical compositions in about 1 hour and about 20-45% of the acetaminophen is released in vitro from the pharmaceutical compositions in about 1 hour in 0.01 N HCl at 50 rpm at 37° C. In another embodiment, about 25-35% of the hydrocodone is released in vitro from the pharmaceutical compositions in about 1 hour and about 25-35% of the acetaminophen is released in vitro from the pharmaceutical compositions in about 1 hour in 0.01 N HCl at 50 rpm at 37° C. Further, in another embodiment, at least 90% of the hydrocodone is released from the pharmaceutical composition in about 8 hours to about 12 hours and at least 60% to about 99% of the acetaminophen is released in vitro from the pharmaceutical compositions in about 6 hours to about 8.5 hours. In another embodiment, at least 90% of the hydrocodone is released from the pharmaceutical composition in about 8 hours to about 11 hours and at least 90% of the acetaminophen is released in vitro from the pharmaceutical compositions in about 8 hours to about 11 hours. In another embodiment, at least 95% of the hydrocodone is released from the pharmaceutical composition in about 9 hours to about 12 hours and at least 95% of the acetaminophen is released in vitro from the pharmaceutical compositions in about 9 hours to about 12 hours. Yet in another embodiment, at least 95% is of the hydrocodone is released from the pharmaceutical composition in about 10 hours to about 12 hours and at least 95% of the acetaminophen is released in vitro from the pharmaceutical compositions in about 10 hours to about 12 hours. In another embodiment, at least 99% of the hydrocodone is released from the pharmaceutical composition in about 11 hours to about 12 hours and at least 99% of the acetaminophen is released in vitro from the pharmaceutical compositions in about 11 hours to about 12 hours. In yet another embodiment, at least 99% of the hydrocodone is released from the pharmaceutical composition in less than about 13 hours and at least 99% of the acetaminophen is released in vitro from the pharmaceutical compositions in less than about 13 hours.

However, when the a slow-release version of the formulation is adapted to be suitable for, or intended for administration to a human, twice daily, as needed, then at least 90% of the hydrocodone is released from the pharmaceutical composition in about 18 hours to about 23 hours and at least 90% of the acetaminophen is released in vitro from the pharmaceutical compositions in about 18 hours to about 23 hours. In another embodiment of the slow release formulation, at least 95% of the hydrocodone is released from the pharmaceutical composition in about 20 hours to about 25 hours and at least 95% of the acetaminophen is released in vitro from the pharmaceutical compositions in about 20 hours to about 25 hours. In another embodiment of the slow release formulation, at least 95% is of the hydrocodone is released from the pharmaceutical composition in about 21 hours to about 22 hours and at least 95% of the acetaminophen is released in vitro from the pharmaceutical compositions in about 21 hours to about 22 hours. In another embodiment of this slow release embodiment, at least 99% of the hydrocodone is released from the pharmaceutical composition in about 22 hours to about 26 hours and at least 99% of the acetaminophen is released in vitro from the pharmaceutical compositions in about 22 hours to about 26 hours. In yet another embodiment of the slow release formulation, at least 99% of the hydrocodone is released from the pharmaceutical composition in less than about 27 hours and at least 99% of the acetaminophen is released in vitro from the pharmaceutical compositions in less than about 27 hours.

In a preferred embodiment, the present invention provides a composition where the core layer comprises an excipient or a mixture of excipients capable of controlling the drug release and the non-core layer comprises an excipient capable of instantly releasing the drug. Further, in a preferred embodiment, the core layer is manufactured by melt-extrusion followed by direct shaping of the drug-containing melt and the non-core layer is spray coated over the core layer. Most preferably, the composition comprises about 500 mg of acetaminophen and about 15 mg of hydrocodone bitartrate pentahemihydrate. In another embodiment, the non-core layer, or the tablet layering may be prepared by another methodology. In this methodology the film-coating layer is separately manufactured by extrusion and the extrudate is shaped into a foil. This foil is introduced into the calendar during manufacturing of the cores. This method is especially suitable for thick layers (saving long spray-coating time) and is a solvent-free process. This technology is also known as the Xellex technology.

In another exemplary embodiment, the present invention provides a pharmaceutical composition having a core and a non-core layer, comprising: (a) an abuse-relevant drug, a pharmaceutically acceptable salt or a hydrate thereof and a non-abuse-relevant drug or a pharmaceutically acceptable salt thereof in the core layer, and (b) a non-abuse-relevant drug, a pharmaceutically acceptable salt or a hydrate thereof in the non-core layer. Preferably, this composition is characterized by at least one of the following features:

i) the amount of abuse-relevant drug that is extracted from the composition by 40% aqueous ethanol within one hour at 37° C. in vitro is less than or equal 1.5 times the amount of the abuse-relevant drug that is extracted by 0.01 N hydrochloric acid in vitro within one hour at 37° C.,
ii) the composition does not break under a force of 150 newtons, preferably 300 newtons, more preferably 450 newtons, yet more preferably 500 newtons as measured by “Pharma Test PTB 501” hardness tester,
iii) the composition releases at least 20% of the abuse-relevant drug and not more than 45% of the abuse-relevant drug during the first hour of in vitro dissolution testing and preferably also during the first hour of in vivo testing,
iv) the composition releases a therapeutically effective dose of the non-abuse relevant drug within 1 to 2 hours after a single dose,
v) the composition releases a therapeutically effective dose of the non-abuse relevant drug and/or the abuse-relevant drug at 1 hour and at 12 hours after a single dose,
vi) in the composition, release of the abuse-relevant drug upon grinding increases by less than 2- to 3-fold, as compared to an intact tablet, when the composition is ground for 1 minute by a coffee-grinder at 20,000-50,000 rpm, in 40% aqueous ethanol for 1 hour at 37° C.,
vii) the composition when ground comprises a particulate size of about 2 cm to about 355 micrometer for about 20% of the fraction, greater than about 63 microns and less than about 355 microns for about 66% of the fraction and less than about 63 microns for about 14% of the fraction, as measured by a sieving test, or
viii) the composition is substantially smooth, wherein the Centre Line Average (CLA) is from about 0.1 to about 0.6, preferably from about 0.1 to about 0.4, and most preferably from about 0.1 to about 0.2.

In this composition, the amount of the abuse-relevant drug that is extracted from the formulation by 40% aqueous ethanol within one hour at 37° C. is about 70% to about 130% of the amount of the drug that is extracted by 0.01 N hydrochloric acid within one hour at 37° C. In another embodiment, the amount of the abuse-relevant drug that is extracted from the formulation by 40% aqueous ethanol within one hour at 37° C. is about 70% to about 90% of the amount of the drug that is extracted by 0.01 N hydrochloric acid within one hour at 37° C. In yet another embodiment, the abuse-relevant drug that is extracted from the formulation by 40% aqueous ethanol within one hour at 37° C. is about 75% to about 90% of the amount of the drug that is extracted by 0.01 N hydrochloric acid within one hour at 37° C.

Another embodiment of the present invention provides a pharmaceutical composition having a core layer and a non-core layer. In this composition the core layer comprises a mixture of: (a) at least one opioid; and (b) at least one rate altering pharmaceutically acceptable polymer, copolymer, or a combination thereof. The non-core layer comprises at least one non-opioid analgesic. Further, these compositions are adapted so as to be useful for oral administration to a human 3, 2, or 1 times daily. Preferably, the core layer further comprises at least one non-opioid analgesic. In a preferred embodiment, the composition is characterized by at least one of the following features:

i) the amount of abuse-relevant drug that is extracted from the composition by 40% aqueous ethanol within one hour at 37° C. in vitro is less than or equal 1.5 times the amount of the abuse-relevant drug that is extracted by 0.01 N hydrochloric acid in vitro within one hour at 37° C.,
ii) the composition does not break under a force of 150 newtons, preferably 300 newtons, more preferably 450 newtons, yet more preferably 500 newtons as measured by “Pharma Test PTB 501” hardness tester,
iii) the composition releases at least 20% of the abuse-relevant drug and not more than 45% of the abuse-relevant drug during the first hour of in vitro dissolution testing and preferably also during the first hour of in vivo testing,
iv) the composition releases a therapeutically effective dose of the non-abuse relevant drug within 1 to 2 hours after a single dose,
v) the composition releases a therapeutically effective dose of the non-abuse relevant drug and/or the abuse-relevant drug at 1 hour and at 12 hours after a single dose,
vi) in the composition, release of the abuse-relevant drug upon grinding increases by less than 2- to 3-fold, as compared to an intact tablet, when the composition is ground for 1 minute by a coffee-grinder at 20,000-50,000 rpm, in 40% aqueous ethanol for 1 hour at 37° C.,
vii) the composition when ground comprises a particulate size of about 2 cm to about 355 micrometer for about 20% of the fraction, greater than about 63 microns and less than about 355 microns for about 66% of the fraction and less than about 63 microns for about 14% of the fraction, as measured by a sieving test, or
viii) the composition is substantially smooth, wherein the Centre Line Average (CLA) is from about 0.1 to about 0.6, preferably from about 0.1 to about 0.4, and most preferably from about 0.1 to about 0.2.

In one embodiment, the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvreturn, pentazocine, phenadoxone, phenazocine, phenomorphan, phenoperidine, piminodine, propiram, propoxyphene, sufentanil, tilidine, and tramadol, and salts, hydrates and mixtures thereof. Further, the non-opioid analgesic is selected from the group consisting of acetaminophen, aspirin, fentaynl, ibuprofen, indomethacin, ketorolac, naproxen, phenacetin, piroxicam, sufentanyl, sunlindac, interferon alpha, and salts, hydrates and mixtures thereof. Preferably, the opioid is hydrocodone and the non-opioid analgesic is acetaminophen or ibuprofen. More preferably, the opioid is hydrocodone and the non-opioid analgesic is acetaminophen.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone of about 0.18 ng/mL/mg to about 1.51 ng/mL/mg, and a plasma concentration at 1 hour Cl for acetaminophen of about 2.34 ng/mL/mg to about 7.24 ng/mL/mg. In preferred embodiments such as Formulation 15, the dosage form produces a Cl for hydrocodone of about 0.32 ng/mL/mg to about 1.51 ng/mL/mg and a Cl for acetaminophen of about 2.34 ng/mL/mg to about 5.50 ng/mL/mg.

In certain other embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone from about 0.30 ng/mL/mg to about 1.06 ng/mL/mg, and a Cl for acetaminophen from about 2.75 ng/mL/mg to about 5.57 ng/mL/mg. In preferred embodiments, the dosage from produces a Cl for hydrocodone from about 0.45 ng/mL/mg to about 1.06 ng/mL/mg and a Cl for acetaminophen from about 2.75 ng/mL/mg to about 4.43 ng/mL/mg.

In certain embodiments, the dosage form produces a combined Cl for hydrocodone and acetaminophen from about 1.18 μg/mL to about 3.63 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.18 μg/mL to about 2.76 μg/mL, after a single dose of 15 mg hydrocodone bitartrate pentahemihydrate and 500 mg of acetaminophen.

In certain embodiments, the dosage form produces a combined Cl for hydrocodone and acetaminophen from about 1.38 μg/mL to about 2.79 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.38 μg/mL to about 2.23 μg/mL, after a single dose of 15 mg hydrocodone bitartrate pentahemihydrate and 500 mg of acetaminophen.

In preferred embodiments, the dosage form produces a combined Cl for hydrocodone and acetaminophen of 1.80±0.42 μg/mL with the 95% confidence interval for the mean value falling between about 1.61 μg/mL to about 2.00 μg/mL, after a single dose of 15 mg hydrocodone bitartrate pentahemihydrate and 500 mg of acetaminophen. The 95% confidence interval of combined Cl for hydrocodone and acetaminophen for the preferred embodiments and the Control overlapped. The 95% confidence interval for the mean value of combined Cl for hydrocodone and acetaminophen for the Control ranged from about 1.46 to 1.96 μg/mL, after administered as a single dose of 15 mg hydrocodone bitartrate pentahemihydrate and 500 mg of acetaminophen to the human patient. The Control provides sufficient plasma levels of opioid and nonopioid analgesic to provide a reduction in pain intensity within about 1 hour after administration.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma profile characterized by a Cmax for hydrocodone from about 0.6 ng/mL/mg to about 1.4 ng/mL/mg and a Cmax for acetaminophen from about 2.8 ng/mL/mg and 7.9 ng/mL/mg after a single dose. In another embodiment, the pharmaceutical composition produces a plasma profile characterized by a Cmax for hydrocodone of about 0.4 ng/mL/mg to about 1.9 ng/mL/mg and a Cmax for acetaminophen of about 2.0 ng/mL/mg to about 10.4 ng/mL/mg after a single dose. In yet another embodiment, the pharmaceutical composition produces a plasma profile characterized by a Cmax for hydrocodone of from about 0.6 ng/mL/mg to about 1.0 ng/mL/mg and a Cmax for acetaminophen of from about 3.0 ng/mL/mg to about 5.2 ng/mL/mg after a single dose.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. When administered to the human patient, the dosage form produces an AUC for hydrocodone of about 9.1 ng*hr/mL/mg to about 19.9 ng*hr/mL/mg and an AUC for acetaminophen of about 28.6 ng*hr/mL/mg to about 59.1 ng*hr/mL/mg. In another embodiment, the dosage form produces an AUC for hydrocodone of about 7.0 ng*hr/mL/mg to about 26.2 ng*hr/mL/mg and an AUC for acetaminophen of about 18.4 ng*hr/mL/mg to about 79.9 ng*hr/mL/mg. In yet another embodiment, the dosage form produces an AUC for hydrocodone of about 11.3 ng*hr/mL/mg to about 18.7 ng*hr/mL/mg and an AUC for acetaminophen of about 28.7 ng*hr/mL/mg to about 53.5 ng*hr/mL/mg. Preferably in this embodiment, the in vitro rate of release of the pharmaceutical composition has a biphasic release profile, and wherein for each phase of the in vitro rate of release is zero order or first order for acetaminophen and zero order or first order for hydrocodone.

When administered to a population of healthy North Americans or Western Europeans, particularly when the formulation is adapted to be suitable for, or intended for, administration to a human every 12 hours as needed, about 20-45% of the hydrocodone is released in vitro from the pharmaceutical compositions in about 1 hour and about 20-45% of the acetaminophen is released in vitro from the pharmaceutical compositions in about 1 hour in 0.01 N HCl at 50 rpm at 37° C. In another embodiment, about 25-35% of the hydrocodone is released in vitro from the pharmaceutical compositions in about 1 hour and about 25-35% of the acetaminophen is released in vitro from the pharmaceutical compositions in about 1 hour in 0.01 N HCl at 50 rpm at 37° C. Further, in another embodiment, at least 90% of the hydrocodone is released from the pharmaceutical composition in about 8 hours to about 12 hours and at least 60% to about 99% of the acetaminophen is released in vitro from the pharmaceutical compositions in about 6 hours to about 8.5 hours. In another embodiment, at least 90% of the hydrocodone is released from the pharmaceutical composition in about 8 hours to about 11 hours and at least 90% of the acetaminophen is released in vitro from the pharmaceutical compositions in about 8 hours to about 11 hours. In another embodiment, at least 95% of the hydrocodone is released from the pharmaceutical composition in about 9 hours to about 12 hours and at least 95% of the acetaminophen is released in vitro from the pharmaceutical compositions in about 9 hours to about 12 hours. Yet in another embodiment, at least 95% is of the hydrocodone is released from the pharmaceutical composition in about 10 hours to about 12 hours and at least 95% of the acetaminophen is released in vitro from the pharmaceutical compositions in about 10 hours to about 12 hours. In another embodiment, at least 99% of the hydrocodone is released from the pharmaceutical composition in about 11 hours to about 12 hours and at least 99% of the acetaminophen is released in vitro from the pharmaceutical compositions in about 11 hours to about 12 hours. In yet another embodiment, at least 99% of the hydrocodone is released from the pharmaceutical composition in less than about 13 hours and at least 99% of the acetaminophen is released in vitro from the pharmaceutical compositions in less than about 13 hours.

However, when the a slow-release version of the formulation is adapted to be suitable for, or intended for administration to a human, twice daily, as needed, then at least 90% of the hydrocodone is released from the pharmaceutical composition in about 18 hours to about 23 hours and at least 90% of the acetaminophen is released in vitro from the pharmaceutical compositions in about 18 hours to about 23 hours. In another embodiment of the slow release formulation, at least 95% of the hydrocodone is released from the pharmaceutical composition in about 20 hours to about 25 hours and at least 95% of the acetaminophen is released in vitro from the pharmaceutical compositions in about 20 hours to about 25 hours. In another embodiment of the slow release formulation, at least 95% is of the hydrocodone is released from the pharmaceutical composition in about 21 hours to about 22 hours and at least 95% of the acetaminophen is released in vitro from the pharmaceutical compositions in about 21 hours to about 22 hours. In another embodiment of this slow release embodiment, at least 99% of the hydrocodone is released from the pharmaceutical composition in about 22 hours to about 26 hours and at least 99% of the acetaminophen is released in vitro from the pharmaceutical compositions in about 22 hours to about 26 hours. In yet another embodiment of the slow release formulation, at least 99% of the hydrocodone is released from the pharmaceutical composition in less than about 27 hours and at least 99% of the acetaminophen is released in vitro from the pharmaceutical compositions in less than about 27 hours.

In a preferred embodiment, the present invention provides a composition where the core layer comprises an excipient capable of controlling the drug release and the non-core layer comprises an excipient capable of instantly releasing the drug. Further, in a preferred embodiment, the core layer is manufactured by melt-extrusion followed by direct shaping of the drug-containing melt and the non-core layer is spray coated over the core layer. Most preferably, the composition comprises about 500 mg of acetaminophen and about 15 mg of hydrocodone bitartrate pentahemihydrate.

In another embodiment, the present invention provides a pharmaceutical composition having a core layer and a non-core layer. In this composition, the core layer comprises a mixture of (a) at least one opioid and at least one first non-opioid analgesic; (b) at least one rate altering pharmaceutically acceptable polymer, copolymer, or a combination thereof. The non-core layer comprises at least one second non-opioid analgesic. Further, the composition is adapted so as to be useful for oral administration to a human 3, 2, or 1 times daily. In this embodiment, preferably, the opioid comprises hydrocodone and the first and the second non-opioid analgesic comprises acetaminophen or ibuprofen. More preferably, the opioid comprises hydrocodone and the first and the second non-opioid analgesic comprises acetaminophen. Further, in this embodiment, the non-core layer comprises: (a) acetaminophen; and (b) at least one rate altering pharmaceutically acceptable polymer, copolymer, or a combination thereof. Preferably, the polymer or copolymer is selected from the group consisting of: hydroxypropyl cellulose, hydroxypropyl methylcellulose, hydroxyethyl cellulose; polymethacrylate, polyvinyl alcohol, polyethylene oxide, and combinations thereof. More preferably, the polymer or copolymer is selected from the group consisting of: hydroxypropyl methylcellulose, and polyvinyl alcohol, or combinations thereof. Yet more preferably, the polymer or copolymer is selected from the group consisting of: polyvinyl alcohol and polyethylene oxide graft copolymers. Further, in this embodiment, the ratio of acetaminophen to the rate controlling polymer or copolymer or combination thereof is about 1:1 to about 10:1. More preferably, the ratio of acetaminophen to the rate controlling polymer or copolymer or combination thereof is about 3:1 to about 5:1. As provided in the present invention, in one preferred embodiment, the non-core layer has at least one of the following characteristics:

(a) substantially does not crack after 3 months at 40° C., 75% relative humidity in induction-sealed HDPE bottles;
(b) substantially dry (not sticky);
provides fast dissolution in 0.01N HCl at 37° C. to expose the core layer
releases at least 80% of the acetaminophen in the non-core layer within 20 minutes of administration to a human patient; or
(e) provides a white pigmentation to the formulation without additional pigments.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone of about 0.18 ng/mL/mg to about 1.51 ng/mL/mg, and a plasma concentration at 1 hour Cl for acetaminophen of about 2.34 ng/mL/mg to about 7.24 ng/mL/mg.

In preferred embodiments such as Formulation 15, the dosage form produces a Cl for hydrocodone of about 0.32 ng/mL/mg to about 1.51 ng/mL/mg and a Cl for acetaminophen of about 2.34 ng/mL/mg to about 5.50 ng/mL/mg.

In certain other embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma concentration at 1 hour (Cl) for hydrocodone from about 0.30 ng/mL/mg to about 1.06 ng/mL/mg, and a Cl for acetaminophen from about 2.75 ng/mL/mg to about 5.57 ng/mL/mg. In preferred embodiments, the dosage from produces a Cl for hydrocodone from about 0.45 ng/mL/mg to about 1.06 ng/mL/mg and a Cl for acetaminophen from about 2.75 ng/mL/mg to about 4.43 ng/mL/mg.

In certain embodiments, the dosage form produces a combined Cl for hydrocodone and acetaminophen from about 1.18 μg/mL to about 3.63 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.18 μg/mL to about 2.76 μg/mL, after a single dose of 15 mg hydrocodone bitartrate pentahemihydrate and 500 mg of acetaminophen.

In certain embodiments, the dosage form produces a combined Cl for hydrocodone and acetaminophen from about 1.38 μg/mL to about 2.79 μg/mL, after a single dose of 15 mg hydrocodone bitartrate pentahemihydrate and 500 mg of acetaminophen. In preferred embodiments, the dosage from produces a combined Cl for hydrocodone and acetaminophen from about 1.38 μg/mL to about 2.23 μg/mL, after a single dose of 15 mg hydrocodone bitartrate pentahemihydrate and 500 mg of acetaminophen.

In preferred embodiments, the dosage form produces a combined Cl for hydrocodone and acetaminophen of 1.80±0.42 μg/mL with the 95% confidence interval for the mean value falling between about 1.61 μg/mL to about 2.00 μg/mL, after a single dose of 15 mg hydrocodone bitartrate pentahemihydrate and 500 mg of acetaminophen. The 95% confidence interval of combined Cl for hydrocodone and acetaminophen for the preferred embodiments and the Control overlapped. The 95% confidence interval for the mean value of combined Cl for hydrocodone and acetaminophen for the Control ranged from about 1.46 to 1.96 μg/mL, after administered as a single dose of 15 mg hydrocodone bitartrate pentahemihydrate and 500 mg of acetaminophen to the human patient. The Control provides sufficient plasma levels of opioid and nonopioid analgesic to provide a reduction in pain intensity within about 1 hour after administration.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. Preferably when administered to a human patient the pharmaceutical composition produces a plasma profile characterized by a Cmax for hydrocodone from about 0.6 ng/mL/mg to about 1.4 ng/mL/mg and a Cmax for acetaminophen from about 2.8 ng/mL/mg and 7.9 ng/mL/mg after a single dose. In another embodiment, the pharmaceutical composition produces a plasma profile characterized by a Cmax for hydrocodone of about 0.4 ng/mL/mg to about 1.9 ng/mL/mg and a Cmax for acetaminophen of about 2.0 ng/mL/mg to about 10.4 ng/mL/mg after a single dose. In yet another embodiment, the pharmaceutical composition produces a plasma profile characterized by a Cmax for hydrocodone of from about 0.6 ng/mL/mg to about 1.0 ng/mL/mg and a Cmax for acetaminophen of from about 3.0 ng/mL/mg to about 5.2 ng/mL/mg after a single dose.

In certain embodiments, the following pharmacokinetic profile is preferably exhibited when the single dose comprises about 15 mg of hydrocodone bitartrate pentahemihydrate and about 500 mg of acetaminophen, administered to the patient, when fasting. When administered to the human patient, the dosage form produces an AUC for hydrocodone of about 9.1 ng*hr/mL/mg to about 19.9 ng*hr/mL/mg and an AUC for acetaminophen of about 28.6 ng*hr/mL/mg to about 59.1 ng*hr/mL/mg. In another embodiment, the dosage form produces an AUC for hydrocodone of about 7.0 ng*hr/mL/mg to about 26.2 ng*hr/mL/mg and an AUC for acetaminophen of about 18.4 ng*hr/mL/mg to about 79.9 ng*hr/mL/mg. In yet another embodiment, the dosage form produces an AUC for hydrocodone of about 11.3 ng*hr/mL/mg to about 18.7 ng*hr/mL/mg and an AUC for acetaminophen of about 28.7 ng*hr/mL/mg to about 53.5 ng*hr/mL/mg. Preferably in this embodiment, the in vitro rate of release of the pharmaceutical composition has a biphasic release profile, and wherein for each phase of the in vitro rate of release is zero order or first order for acetaminophen and zero order or first order for hydrocodone.

When administered to a population of healthy North Americans or Western Europeans, particularly when the formulation is adapted to be suitable for, or intended for, administration to a human every 12 hours as needed, about 20-45% of the hydrocodone is released in vitro from the pharmaceutical compositions in about 1 hour and about 20-45% of the acetaminophen is released in vitro from the pharmaceutical compositions in about 1 hour in 0.01 N HCl at 50 rpm at 37° C. In another embodiment, about 25-35% of the hydrocodone is released in vitro from the pharmaceutical compositions in about 1 hour and about 25-35% of the acetaminophen is released in vitro from the pharmaceutical compositions in about 1 hour in 0.01 N HCl at 50 rpm at 37° C. Further, in another embodiment, at least 90% of the hydrocodone is released from the pharmaceutical composition in about 8 hours to about 12 hours and at least 60% to about 99% of the acetaminophen is released in vitro from the pharmaceutical compositions in about 6 hours to about 8.5 hours. In another embodiment, at least 90% of the hydrocodone is released from the pharmaceutical composition in about 8 hours to about 11 hours and at least 90% of the acetaminophen is released in vitro from the pharmaceutical compositions in about 8 hours to about 11 hours. In another embodiment, at least 95% of the hydrocodone is released from the pharmaceutical composition in about 9 hours to about 12 hours and at least 95% of the acetaminophen is released in vitro from the pharmaceutical compositions in about 9 hours to about 12 hours. Yet in another embodiment, at least 95% is of the hydrocodone is released from the pharmaceutical composition in about 10 hours to about 12 hours and at least 95% of the acetaminophen is released in vitro from the pharmaceutical compositions in about 10 hours to about 12 hours. In another embodiment, at least 99% of the hydrocodone is released from the pharmaceutical composition in about 11 hours to about 12 hours and at least 99% of the acetaminophen is released in vitro from the pharmaceutical compositions in about 11 hours to about 12 hours. In yet another embodiment, at least 99% of the hydrocodone is released from the pharmaceutical composition in less than about 13 hours and at least 99% of the acetaminophen is released in vitro from the pharmaceutical compositions in less than about 13 hours.

However, when the a slow-release version of the formulation is adapted to be suitable for, or intended for administration to a human, twice daily, as needed, then at least 90% of the hydrocodone is released from the pharmaceutical composition in about 18 hours to about 23 hours and at least 90% of the acetaminophen is released in vitro from the pharmaceutical compositions in about 18 hours to about 23 hours. In another embodiment of the slow release formulation, at least 95% of the hydrocodone is released from the pharmaceutical composition in about 20 hours to about 25 hours and at least 95% of the acetaminophen is released in vitro from the pharmaceutical compositions in about 20 hours to about 25 hours. In another embodiment of the slow release formulation, at least 95% is of the hydrocodone is released from the pharmaceutical composition in about 21 hours to about 22 hours and at least 95% of the acetaminophen is released in vitro from the pharmaceutical compositions in about 21 hours to about 22 hours. In another embodiment of this slow release embodiment, at least 99% of the hydrocodone is released from the pharmaceutical composition in about 22 hours to about 26 hours and at least 99% of the acetaminophen is released in vitro from the pharmaceutical compositions in about 22 hours to about 26 hours. In yet another embodiment of the slow release formulation, at least 99% of the hydrocodone is released from the pharmaceutical composition in less than about 27 hours and at least 99% of the acetaminophen is released in vitro from the pharmaceutical compositions in less than about 27 hours.

In a preferred embodiment, the present invention provides a composition where the core layer comprises an excipient capable of controlling the drug release and the non-core layer comprises an excipient capable of instantly releasing the drug. Further, in a preferred embodiment, the core layer is manufactured by melt-extrusion followed by direct shaping of the drug-containing melt and the non-core layer is spray coated over the core layer. Most preferably, the composition comprises about 500 mg of acetaminophen and about 15 mg of hydrocodone bitartrate pentahemihydrate.

In a preferred embodiment, the composition is characterized by at least one of the following features:

i) the amount of abuse-relevant drug that is extracted from the composition by 40% aqueous ethanol within one hour at 37° C. in vitro is less than or equal 1.5 times the amount of the hydrocodone that is extracted by 0.01 N hydrochloric acid in vitro within one hour at 37° C.,
ii) the composition does not break under a force of 150 newtons, preferably 300 newtons, more preferably 450 newtons, yet more preferably 500 newtons as measured by “Pharma Test PTB 501” hardness tester,
iii) the composition releases at least 20% of the hydrocodone and not more than 45% of the hydrocodone during the first hour of in vitro dissolution testing and preferably also during the first hour of in vivo testing,
iv) the composition releases a therapeutically effective dose of the acetaminophen within 1 to 2 hours after a single dose,
v) the composition releases a therapeutically effective dose of the acetaminophen and/or the abuse-relevant drug at 1 hour and at 12 hours after a single dose,
vi) in the composition, release of the hydrocodone upon grinding increases by less than 2- to 3-fold, as compared to an intact tablet, when the composition is ground for 1 minute by a coffee-grinder at 20,000-50,000 rpm, in 40% aqueous ethanol for 1 hour at 37° C.,
vii) the composition when ground comprises a particulate size of about 2 cm to about 355 micrometer for about 20% of the fraction, greater than about 63 microns and less than about 355 microns for about 66% of the fraction and less than about 63 microns for about 14% of the fraction, as measured by a sieving test, or
viii) the composition is substantially smooth, wherein the Centre Line Average (CLA) is from about 0.1 to about 0.6, preferably from about 0.1 to about 0.4, and most preferably from about 0.1 to about 0.2.

The foregoing detailed description and accompanying examples are merely illustrative and not intended as limitations upon the scope of the invention, which is defined solely by the appended claims and their equivalents. Various changes and modifications to the disclosed embodiments will be apparent to those skilled in the art and are part of the present invention. Such changes and modifications, including without limitation those relating to the chemical structures, substituents, derivatives, intermediates, syntheses, formulations and/or methods of use of the invention, can be made without departing from the spirit and scope thereof.

1. A pharmaceutical composition having a core and a non-core layer, comprising:
(a) hydrocodone, a pharmaceutically acceptable salt or a hydrate thereof, and
(b) acetaminophen or ibuprofen,
wherein at least 75% all of the hydrocodone, pharmaceutically acceptable salt or hydrate thereof is in the core,
wherein the acetaminophen or the ibuprofen is the non-core layer, and
wherein the composition is adapted so as to be useful for oral administration to a human 3,2, or 1 times daily.

2. The composition of claim 1, wherein greater than 90% of the hydrocodone, pharmaceutically acceptable salt or hydrate thereof is in the core.

3. The composition of claim 1, wherein substantially all of the hydrocodone, pharmaceutically acceptable salt or hydrate thereof is in the core.

4. (canceled)

5. The composition of claim 1, further wherein the core further comprises acetaminophen or ibuprofen.

6. The composition of claim 1, wherein when administered to the human patient the pharmaceutical composition produces a plasma profile characterized by a Cmax for hydrocodone from about 0.6 ng/mL/mg to about 1.4 ng/mL/mg and a Cmax for acetaminophen from about 2.8 ng/mL/mg and 7.9 ng/mL/mg after a single dose.

7. The composition of claim 1, wherein when administered to the human patient, the pharmaceutical composition produces a plasma profile characterized by a Cmax for hydrocodone of about 0.4 ng/mL/mg to about 1.9 ng/mL/mg and a Cmax for acetaminophen of about 2.0 ng/mL/mg to about 10.4 ng/mL/mg after a single dose.

8. The composition of claim 1, wherein when administered to the human patient, the pharmaceutical composition produces a plasma profile characterized by a Cmax for hydrocodone of from about 0.6 ng/mL/mg to about 1.0 ng/mL/mg and a Cmax for acetaminophen of from about 3.0 ng/mL/mg to about 5.2 ng/mL/mg after a single dose.

9. The composition of claim 1, wherein when administered to the human patient, the dosage form produces an AUC for hydrocodone of about 9.1 ng*hr/mL/mg to about 19.9 ng*hr/mL/mg and an AUC for acetaminophen of about 28.6 ng*hr/mL/mg to about 59.1 ng*hr/mL/mg.

10. The composition of claim 1, wherein when administered to the human patient, the dosage form produces an AUC for hydrocodone of about 7.0 ng*hr/mL/mg to about 26.2 ng*hr/mL/mg and an AUC for acetaminophen of about 18.4 ng*hr/mL/mg to about 79.9 ng*hr/mL/mg.

11. The composition of claim 1, wherein when administered to the human patient, the dosage form produces an AUC for hydrocodone of about 11.3 ng*hr/mL/mg to about 18.7 ng*hr/mL/mg and an AUC for acetaminophen of about 28.7 ng*hr/mL/mg to about 53.5 ng*hr/mL/mg.

12. The composition of claim 1, wherein the in vitro rate of release of the pharmaceutical composition has a biphasic release profile, and wherein for each phase of the in vitro rate of release is zero order or first order for acetaminophen and zero order or first order for hydrocodone.

13. The composition of claim 1, wherein about 20-45% of the hydrocodone is released in vitro from the pharmaceutical compositions in about 1 hour and about 20-45% of the acetaminophen is released in vitro from the pharmaceutical compositions in about 1 hour in 0.01 N HCl at 50 rpm at 37° C.

14. The composition of claim 1, wherein about 25-35% of the hydrocodone is released in vitro from the pharmaceutical compositions in about 1 hour and about 25-35% of the acetaminophen is released in vitro from the pharmaceutical compositions in about 1 hour in 0.01 N HCl at 50 rpm at 37° C.

15. The composition of claim 1, wherein at least 90% of the hydrocodone is released from the pharmaceutical composition in about 8 hours to about 12 hours and at least 60% to about 99% of the acetanlinophen is released in vitro from the pharmaceutical compositions in about 6 hours to about 8.5 hours.

16. The composition of claim 1, wherein at least 90% of the hydrocodone is released from the pharmaceutical composition in about 18 hours to about 23 hours and at least 90% of the acetaminophen is released in vitro from the pharmaceutical compositions in about 18 hours to about 23 hours.

17. The composition of claim 1, wherein at least 90% of the hydrocodone is released from the pharmaceutical composition in about 8 hours to about 11 hours and at least 90% of the acetaminophen is released in vitro from the pharmaceutical compositions in about 8 hours to about 11 hours.

18. The composition of claim 1, wherein at least 95% of the hydrocodone is released from the pharmaceutical composition in about 9 hours to about 12 hours and at least 95% of the acetaminophen is released in vitro from the pharmaceutical compositions in about 9 hours to about 12 hours.

19. The composition of claim 1, wherein at least 95% is of the hydrocodone is released from the pharmaceutical composition in about 10 hours to about 12 hours and at least 95% of the acetaminophen is released in vitro from the pharmaceutical compositions in about 10 hours to about 12 hours.

20. The composition of claim 1, wherein at least 95% of the hydrocodone is released from the pharmaceutical composition in about 20 hours to about 25 hours and at least 95% of the acetaminophen is released in vitro from the pharmaceutical compositions in about 20 hours to about 25 hours.

21. The composition of claim 1, wherein at least 95% is of the hydrocodone is released from the pharmaceutical composition in about 21 hours to about 22 hours and at least 95% of the acetaminophen is released in vitro from the pharmaceutical compositions in about 21 hours to about 22 hours.

22. The composition of claim 1, wherein at least 99% of the hydrocodone is released from the pharmaceutical composition in about 11 hours to about 12 hours and at least 99% of the acetaminophen is released in vitro from the pharmaceutical compositions in about 11 hours to about 12 hours.

23. The composition of claim 1, wherein at least 99% of the hydrocodone is released from the pharmaceutical composition in less than about 13 hours and at least 99% of the acetaminophen is released in vitro from the pharmaceutical compositions in less than about 13 hours.

24. The composition of claim 1, wherein at least 99% of the hydrocodone is released from the pharmaceutical composition in about 22 hours to about 26 hours and at least 99% of the acetaminophen is released in vitro from the pharmaceutical compositions in about 22 hours to about 26 hours.

25. The composition of claim 1, wherein at least 99% of the hydrocodone is released from the pharmaceutical composition in less than about 27 hours and at least 99% of the acetaminophen is released in vitro from the pharmaceutical compositions in less than about 27 hours.

26. The composition of claim 1, wherein the core layer comprises an excipient capable of controlling the drug release and the non-core layer comprises an excipient capable of instantly releasing the drug.

27. The composition of claim 1, wherein the core layer is manufactured by melt-extrusion followed by direct shaping of the drug containing melt and the non-core layer is spray coated over the core layer.

28. The composition of claim 1, wherein the composition comprises about 500 mg of acetaminophen and about 15 mg of hydrocodone bitartrate pentahemihydrate.

29. A pharmaceutical composition having a core and a non-core layer, comprising:
(a) an abuse-relevant drug, a pharmaceutically acceptable salt or a hydrate thereof and a non-abuse-relevant drug or a pharmaceutically acceptable salt thereof in the core layer, and
(b) a non-abuse-relevant drug, a pharmaceutically acceptable salt or a hydrate thereof in the non-core layer,
wherein the composition is adapted so as to “be useful for oral administration to a human 3,2, or 1 times daily; and
wherein the composition is characterized by at least one of the following features:
i) the amount of abuse-relevant drug that is extracted from the composition by 40% aqueous ethanol within one hour at 37° C. in vitro is less than or equal 1.5 times the amount of the abuse relevant drug that is extracted by 0.01 N hydrochloric acid in vitro within one hour at 37° C.,
ii) the composition does not break under a force of 150 newtons, preferably 300 newtons, more preferably 450 newtons, yet more preferably 500 newtons as measured by “Pharma Test PTB 50 1” hardness tester,
iii) the composition releases at least 20% of the abuse-relevant drug and not more than 45% of the abuse-relevant drug during the first hour of in vitro dissolution testing and preferably also during the first hour of in vivo testing,
iv) the composition releases a therapeutically effective dose of the non-abuse relevant drug within 1 to 2 hours after a single dose,
v) the composition releases a therapeutically effective dose of the non-abuse relevant drug and/or the abuse-relevant drug at 1 hour and at 12 hours after a single dose,
vi) in the composition, release of the abuse-relevant drug upon grinding increases by less than 2- to 3-fold, as compared to an intact tablet, when the composition is ground for 1 minute by a coffee-grinder at 20,000-50,000 rpm, in 40% aqueous ethanol for 1 hour at 37° C.,
vii) the composition when ground comprises a particulate size of about 2 cm to about 355 micrometer for about 20% of the fraction, greater than about 63 microns and less than about 355 microns for about 66% of the fraction and less than about 63 microns for about 14% of the fraction, as measured by a sieving test, or
viii) the composition is substantially smooth, wherein the Centre Line Average (CLA) is from about 0.1 to about 0.6, preferably from about 0.1 to about 0.4, and most preferably from about 0.1 to about 0.2.

30. The composition of claim 29, wherein the amount of the abuse-relevant drug that is extracted from the formulation by 40% aqueous ethanol within one hour at 37° C. is about 70% to about 90% of the amount of the drug that is extracted by 0.01 N hydrochloric acid within one hour at 37° C.

31. The composition of claim 29, wherein the amount of the abuse-relevant drug that is extracted from the formulation by 40% aqueous ethanol within one hour at 37° C. is about 70% to about 130% of the amount of the drug that is extracted by 0.01 N hydrochloric acid within one hour at 37° C.

32. The composition of claim 29, wherein the amount of the abuse-.relevant drug that is extracted from the formulation by 40% aqueous ethanol within one hour at 37° C. is about 75% to about 90% of the amount of the drug that is extracted by 0.01 N hydrochloric acid within one hour at 37° C.

33. A pharmaceutical composition having a core layer and a non-core layer,
(A) wherein the core layer comprises a mixture of:
(a) at least one opioid;
(b) at least one rate altering pharmaceutically acceptable polymer, copolymer, or a combination thereof;
(B) wherein the non-core layer comprises at least one non-opioid analgesic; and
(C) wherein the composition is adapted so as to be useful for oral administration to a human 3,2, or 1 times daily.

34. The composition of claim 33, wherein the core layer further comprises at least one non-opioid analgesic.

35. The composition of claim 33, wherein the composition is characterized by at least one of the following features:
i) the amount of abuse-relevant drug that is extracted from the composition by 40% aqueous ethanol within one hour at 37° C. in vitro is less than or equal 1.5 times the amount of the abuse-relevant drug that is extracted by 0.01 N hydrochloric acid in vitro within one hour at 37° C.,
ii) the composition does not break under a force of 150 newtons, preferably 300 newtons, more preferably 450 newtons, yet more preferably 500 newtons as measured by “Pharma Test PTB 501” hardness tester,
iii) the composition releases at least 20% of the abuse-relevant drug and not more than 45% of the abuse-relevant drug during the first hour of in vitro dissolution testing and preferably also during the first hour of in vivo testing,
iv) the composition releases a therapeutically effective dose of the non-abuse relevant drug within 1 to 2 hours after a single dose,
v) the composition releases a therapeutically effective dose of the non-abuse relevant drug and/or the abuse-relevant drug at 1 hour and at 12 hours after a single dose,
vi) in the composition, release of the abuse-relevant drug upon grinding increases by less than 2- to 3-fold, as compared to an intact tablet, when the composition is ground for 1 minute by a coffee-grinder at 20,000-50,000 rpm, in 40% aqueous ethanol for 1 hour at 37° C.,
vii) the composition when ground comprises a particulate size of about 2 cm to about 355 micrometer for about 20% of the fraction, greater than about 63 microns and less than about 355 microns for about 66% of the fraction and less than about 63 microns for about 14% of the fraction, as measured by a sieving test, or
viii) the composition is substantially smooth, wherein the Centre Line Average (CLA) is from about 0.1 to about 0.6, preferably from about 0.1 to about 0.4, and most preferably from about 0.1 to about 0.2.

36. The composition of claim 33, wherein the opioid is selected from the group consisting of alfentanil, allylprodine, alphaprodine, anileridine, benzyl morphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvreturn, pentazocine, phenadoxone, phenazocine, phenomorphan, phenoperidine, piminodine, propiram, propoxyphene, sufentanil, tilidine, and tramadol, and salts, hydrates and mixtures thereof and the non-opioid analgesic is selected from the group consisting of acetaminophen, aspirin, fentaynl, ibuprofen, indomethacin, ketorolac, naproxen, phenacetin, piroxicam, sufentanyl, sunlindac, interferon alpha, and salts, hydrates and mixtures thereof.

37. The composition of claim 33, wherein the opioid is hydrocodone and the nonopioid analgesic is acetaminophen or ibuprofen.

38. (canceled)

39. The composition of claim 33, wherein when administered to the human patient, the pharmaceutical composition produces a plasma profile characterized by a Cmax for hydrocodone from about 0.6 ng/mL/mg to about 1.4 ng/mL/mg and a Cmax for acetaminophen from about 2.8 ng/mL/mg and 7.9 ng/mL/mg after a single dose.

40. The composition of claim 33, wherein when administered to the human patient, the pharmaceutical composition produces a plasma profile characterized by a Cmax for hydrocodone of about 0.4 ng/mL/mg to about 1.9 ng/mL/mg and a Cmax for acetaminophen of about 2.0 ng/mL/mg to about 10.4 ng/mL/mg after a single dose.

41. The composition of claim 33, wherein when administered to the human patient, the pharmaceutical composition produces a plasma profile characterized by a Cmax for hydrocodone of from about 0.6 ng/mL/mg to about 1.0 ng/mL/mg and a Cmax for acetaminophen of from about 3.0 ng/mL/mg to about 5.2 ng/mL/mg after a single dose.

42. The composition of claim 33, wherein when administered to the human patient, the dosage form produces an AUC for hydrocodone of about 9.1 ng*hr/mL/mg to about 19.9 ng*hr/mL/mg and an AUC for acetaminophen of about 28.6 ng*hr/mL/mg to about 59.1 ng*hr/mL/mg.

43. The composition of claim 33, wherein when administered to the human patient, the dosage form produces an AUC for hydro co done of about 7.0 ng*hr/mL/mg to about 26.2 ng*hr/mL/mg and an AUC for acetaminophen of about 18.4 ng*hr/mL/mg to about 79.9 ng*hr/mL/mg.

44. The composition of claim 33, wherein when administered to the human patient, the dosage form produces an AUC for hydrocodone of about 11.3 ng*hr/mL/mg to about 18.7 ng*hr/mL/mg and an AUC for acetaminophen of about 28.7 ng*hr/mL/mg to about 53.5 ng*hr/mL/mg.

45. The composition of claim 33, wherein the in vitro rate of release of the pharmaceutical composition has a biphasic release profile, and wherein for each phase of the in vitro rate of release is zero order or first order for acetaminophen and zero order or first order for hydrocodone.

46. The composition of claim 33, wherein about 20-45% of the hydrocodone is released in vitro from the pharmaceutical compositions in about 1 hour and about 20-45% of the acetaminophen is released in vitro from the pharmaceutical compositions in about 1 hour in 0.01 N HCl at 50 rpm at 37° C.

47. The composition of claim 33, wherein about 25-35% of the hydrocodone is released in vitro from the pharmaceutical compositions in about 1 hour and about 25-35% of the acetaminophen is released in vitro from the pharmaceutical compositions in about 1 hour in 0.01 N HCl at 50 rpm at 37° C.

48. The composition of claim 33, wherein about 90% of the hydrocodone is released from the pharmaceutical composition in about 8 hours to about 12 hours and at least 60% to about 99% of the acetaminophen is released in vitro from the pharmaceutical compositions in about 6 hours to about 8.5 hours.

49. The composition of claim 33, wherein about 90% of the hydrocodone is released from the pharmaceutical composition in about 18 hours to about 23 hours and about 90% of the acetaminophen is released in vitro from the pharmaceutical compositions in about 18 hours to about 23 hours.

50. The composition of claim 33, wherein about 90% of the hydrocodone is released from the pharmaceutical composition in about 8 hours to about 11 hours and about 90% of the acetaminophen is released in vitro from the pharmaceutical compositions in about 8 hours to about 11 hours.

51. The composition of claim 33, wherein about 95% of the hydrocodone is released from the pharmaceutical composition in about 9 hours to about 12 hours and about 95% of the acetaminophen is released in vitro from the pharmaceutical compositions in about 9 hours to about 12 hours.

52. The composition of claim 33, wherein about 95% is of the hydrocodone is released from the pharmaceutical composition in about 10 hours to about 12 hours and about 95% of the acetaminophen is released in vitro from the pharmaceutical compositions in about 10 hours to about 12 hours.

53. The composition of claim 33, wherein about 95% of the hydrocodone is released from the pharmaceutical composition in about 20 hours to about 25 hours and about 95% of the acetaminophen is released in vitro from the pharmaceutical compositions in about 20 hours to about 25 hours.

54. The composition of claim 33, wherein about 95% is of the hydrocodone is released from the pharmaceutical composition in about 21 hours to about 22 hours and about 95% of the acetaminophen is released in vitro from the pharmaceutical compositions in about 21 hours to about 22 hours.

55. The composition of claim 33, wherein about 99% of the hydrocodone is released from the pharmaceutical composition in about 11 hours to about 12 hours and about 99% of the acetaminophen is released in vitro from the pharmaceutical compositions in about 11 hours to about 12 hours.

56. The composition of claim 33, wherein about 99% of the hydrocodone is released from the pharmaceutical composition in less than about 13 hours and about 99% of the acetaminophen is released in vitro from the pharmaceutical compositions in less than about 13 hours.

57. The composition of claim 33, wherein about 99% of the hydrocodone is released from the pharmaceutical composition in about 22 hours to about 26 hours and about 99% of the acetaminophen is released in vitro from the pharmaceutical compositions in about 22 hours to about 26 hours.

58. The composition of claim 33, wherein about 99% of the hydrocodone is released from the pharmaceutical composition in less than about 27 hours and about 99% of the acetaminophen is released in vitro from the pharmaceutical compositions in less than about 27 hours.

59. The composition of claim 33, wherein the core layer comprises an excipient capable of controlling the drug release and the non-core layer comprises an excipient capable of instantly releasing the drug.

60. The composition of claim 33, wherein the core layer is manufactured by melt-extrusion followed by direct shaping of the drug-containing melt and the non-core layer is spray coated over the core layer.

61.-92. (canceled)
